SEQUENCE LISTING (55 sequences)

SEQ ID NO: 1 - protein, 245 aa, Homo sapiens
MAAAIASSLIRQKRQAREREKSNACKCVSSPSKGKTSCDKNKLNVFSRVKLFGSKKRRRRRPEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 2 - DNA, 2705 bp, Homo sapiens
gtgccgcgcccagagcagcagcaacagcgaagatgcgaggccattacctgtttgatccctgtcggaaacctggcacgggccaacttttcccgattatcacgccaagaagttgcaaggactagtcgaagactcggaggggccagggcgagggcgcgctcccccgcgcgctgcctcgtccctcctccgtccggccgcccgagctcccggcctctctcccgcccgcgctcactccctccgcccgcctccctcctctggcccccatcagaagggcaacagggcgagggggtccggcgaaattcggaccggagcagctggacatgcacggtgtccgccgggcgcaggggccgaccacacgcagtcgcgcagttcagcatccgcgtgccagtctcgcccgcgatcccgggcccggggctgtggcgtcgactccgacccaggcagccagcagcccgcgcgggagccggaccgccgccggaggagctcggacggcatgctgagccccctccttggctgaagcccgagtgcggagaagcccgggcaaacgcaggctaaggagaccaaagcggcgaagtcgcgagacagcggacaagcagcggaggagaaggaggaggaggcgaacccagagaggggcagcaaaagaagcggtggtggtgggcgtcgtggccatggcggcggctatcgccagctcgctcatccgtcagaagaggcaagcccgcgagcgcgagaaatccaacgcctgcaagtgtgtcagcagccccagcaaaggcaagaccagctgcgacaaaaacaagttaaatgtcttttcccgggtcaaactcttcggctccaagaagaggcgcagaagaagaccagagcctcagcttaagggtatagttaccaagctatacagccgacaaggctaccacttgcagctgcaggcggatggaaccattgatggcaccaaagatgaggacagcacttacactctgtttaacctcatccctgtgggtctgcgagtggtggctatccaaggagttcaaaccaagctgtacttggcaatgaacagtgagggatacttgtacacctcggaacttttcacacctgagtgcaaattcaaagaatcagtgtttgaaaattattatgtgacatattcatcaatgatataccgtcagcagcagtcaggccgagggtggtatctgggtctgaacaaagaaggagagatcatgaaaggcaaccatgtgaagaagaacaagcctgcagctcattttctgcctaaaccactgaaagtggccatgtacaaggagccatcactgcacgatctcacggagttctcccgatctggaagcgggaccccaaccaagagcagaagtgtctctggcgtgctgaacggaggcaaatccatgagccacaatgaatcaacgtagccagtgagggcaaaagaagggctctgtaacagaaccttacctccaggtgctgttgaattcttctagcagtccttcacccaaaagttcaaatttgtcagtgacatttaccaaacaaacaggcagagttcactattctatctgccattagaccttcttatcatccatactaaagccccattatttagattgagcttgtgcataagaatgccaagcattttagtgaactaaatctgagagaaggactgccaaattttctcatgatctcacctatactttggggatgataatccaaaagtatttcacagcactaatgctgatcaaaatttgctctcccaccaagaaaatgtaaaagaccacaattgttcttcaaaaacaaacaaaacaaaacaaaacaaaattaactgcttaaatgttttgtcggggcaaacaaaattatgtgaattgtgttgttttcttggcttgatgttttctatctacgcttgattcacatgtactcttttctttggcatagtgcaactttatgatttctgaaattcaatggttctattgactttttgcgtcacttaatccaaatcaaccaaattcagggttgaatctgaattggcttctcaggctcaaggtaacagtgttcttgtggtttgaccaattgtttttctttctttttttttttttttagatttgtggtattctggtcaagttattgtgctgtactttgtgcgtagaaattgagttgtattgtcaaccccagtcagtaaagagaacttcaaaaaattatcctcaagtgtagatttctcttaattccatttgtgtatcatgttaaactattgttgtggcttcttgtgtaaagacaggaactgtggaactgtgatgttgtcttttgtgttgttaaaataagaaatgtcttatctgtatatgtatgagtcttcctgtcattgtatttggcacatgaatattgtgtacaaggaattgttaagactggttttccctcaacaacatatattatacttgctactggaaaagtgtttaagacttagctaggtttccatttagatcttcatatctgttgcatggaagaaagttgggttcttggcatagagttgcatgatatgtaagattttgtgcattcataattgttaaaaatctgtgttccaaaagtggacatagcatgtacaggcagttttctgtcctgtgcacaaaaagtttaaaaaagttgtttaatatttgttgttgtatacccaaatacgcaccgaataaactctttatattcattcaaagaaaaaaaaaaaaaaaaaaaaaaaaaaaaaa

SEQ ID NO: 3 - protein, 255 aa, Homo sapiens
MSGKVTKPKEEKDASKVLDDAPPGTQEYIMLRQDSIQSAELKKKESPFRAKCHEIFCCPLKQVHHKENTEPEEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 4 - DNA, 2340 bp, Homo sapiens
gtggctctctaggaccggagagttctttggaaggagagcgcgagcgagggagcgggcgagctccgagggggtgtgggtgtagggagagagagaaagagagcaggcagcggcggcggcggcagcggtggggaaaagcggattccgccccgaaccacaccgaggggagctcgtggtcgagacttgccgccctaagcactctcccaagtccgacccgctcggcgaggacttccgtcttctgagcgaaccttgtcaagcaagctgggatctatgagtggaaaggtgaccaagcccaaagaggagaaagatgcttctaaggttctggatgacgccccccctggcacacaggaatacattatgttacgacaagattccatccaatctgcggaattaaagaaaaaagagtccccctttcgtgctaagtgtcacgaaatcttctgctgcccgctgaagcaagtacaccacaaagagaacacagagccggaagagcctcagcttaagggtatagttaccaagctatacagccgacaaggctaccacttgcagctgcaggcggatggaaccattgatggcaccaaagatgaggacagcacttacactctgtttaacctcatccctgtgggtctgcgagtggtggctatccaaggagttcaaaccaagctgtacttggcaatgaacagtgagggatacttgtacacctcggaacttttcacacctgagtgcaaattcaaagaatcagtgtttgaaaattattatgtgacatattcatcaatgatataccgtcagcagcagtcaggccgagggtggtatctgggtctgaacaaagaaggagagatcatgaaaggcaaccatgtgaagaagaacaagcctgcagctcattttctgcctaaaccactgaaagtggccatgtacaaggagccatcactgcacgatctcacggagttctcccgatctggaagcgggaccccaaccaagagcagaagtgtctctggcgtgctgaacggaggcaaatccatgagccacaatgaatcaacgtagccagtgagggcaaaagaagggctctgtaacagaaccttacctccaggtgctgttgaattcttctagcagtccttcacccaaaagttcaaatttgtcagtgacatttaccaaacaaacaggcagagttcactattctatctgccattagaccttcttatcatccatactaaagccccattatttagattgagcttgtgcataagaatgccaagcattttagtgaactaaatctgagagaaggactgccaaattttctcatgatctcacctatactttggggatgataatccaaaagtatttcacagcactaatgctgatcaaaatttgctctcccaccaagaaaatgtaaaagaccacaattgttcttcaaaaacaaacaaaacaaaacaaaacaaaattaactgcttaaatgttttgtcggggcaaacaaaattatgtgaattgtgttgttttcttggcttgatgttttctatctacgcttgattcacatgtactcttttctttggcatagtgcaactttatgatttctgaaattcaatggttctattgactttttgcgtcacttaatccaaatcaaccaaattcagggttgaatctgaattggcttctcaggctcaaggtaacagtgttcttgtggtttgaccaattgtttttctttctttttttttttttttagatttgtggtattctggtcaagttattgtgctgtactttgtgcgtagaaattgagttgtattgtcaaccccagtcagtaaagagaacttcaaaaaattatcctcaagtgtagatttctcttaattccatttgtgtatcatgttaaactattgttgtggcttcttgtgtaaagacaggaactgtggaactgtgatgttgtcttttgtgttgttaaaataagaaatgtcttatctgtatatgtatgagtcttcctgtcattgtatttggcacatgaatattgtgtacaaggaattgttaagactggttttccctcaacaacatatattatacttgctactggaaaagtgtttaagacttagctaggtttccatttagatcttcatatctgttgcatggaagaaagttgggttcttggcatagagttgcatgatatgtaagattttgtgcattcataattgttaaaaatctgtgttccaaaagtggacatagcatgtacaggcagttttctgtcctgtgcacaaaaagtttaaaaaagttgtttaatatttgttgttgtatacccaaatacgcaccgaataaactctttatattcattcaaagaaaaaaaaaaaaaaaaaaaaaaaaaaaaaa

SEQ ID NO: 5 - protein, 226 aa, Homo sapiens
MLRQDSIQSAELKKKESPFRAKCHEIFCCPLKQVHHKENTEPEEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 6 - DNA, 2450 bp, Homo sapiens
gtggctctctaggaccggagagttctttggaaggagagcgcgagcgagggagcgggcgagctccgagggggtgtgggtgtagggagagagagaaagagagcaggcagcggcggcggcggcagcggtggggaaaagcggattccgccccgaaccacaccgaggggagctcgtggtcgagacttgccgccctaagcactctcccaagtccgacccgctcggcgaggacttccgtcttctgagcgaaccttgtcaagcaagctgggatctatgagtggaaaggtgaccaagcccaaagaggagaaagatgcttctaagggagtttctctgcacaagctctctgtttgcctgctgtcgtccacataagatgtgacttgctcctgcttgccttcctccatgattgtgaggcctccccagccacgtggaactttctggatgacgccccccctggcacacaggaatacattatgttacgacaagattccatccaatctgcggaattaaagaaaaaagagtccccctttcgtgctaagtgtcacgaaatcttctgctgcccgctgaagcaagtacaccacaaagagaacacagagccggaagagcctcagcttaagggtatagttaccaagctatacagccgacaaggctaccacttgcagctgcaggcggatggaaccattgatggcaccaaagatgaggacagcacttacactctgtttaacctcatccctgtgggtctgcgagtggtggctatccaaggagttcaaaccaagctgtacttggcaatgaacagtgagggatacttgtacacctcggaacttttcacacctgagtgcaaattcaaagaatcagtgtttgaaaattattatgtgacatattcatcaatgatataccgtcagcagcagtcaggccgagggtggtatctgggtctgaacaaagaaggagagatcatgaaaggcaaccatgtgaagaagaacaagcctgcagctcattttctgcctaaaccactgaaagtggccatgtacaaggagccatcactgcacgatctcacggagttctcccgatctggaagcgggaccccaaccaagagcagaagtgtctctggcgtgctgaacggaggcaaatccatgagccacaatgaatcaacgtagccagtgagggcaaaagaagggctctgtaacagaaccttacctccaggtgctgttgaattcttctagcagtccttcacccaaaagttcaaatttgtcagtgacatttaccaaacaaacaggcagagttcactattctatctgccattagaccttcttatcatccatactaaagccccattatttagattgagcttgtgcataagaatgccaagcattttagtgaactaaatctgagagaaggactgccaaattttctcatgatctcacctatactttggggatgataatccaaaagtatttcacagcactaatgctgatcaaaatttgctctcccaccaagaaaatgtaaaagaccacaattgttcttcaaaaacaaacaaaacaaaacaaaacaaaattaactgcttaaatgttttgtcggggcaaacaaaattatgtgaattgtgttgttttcttggcttgatgttttctatctacgcttgattcacatgtactcttttctttggcatagtgcaactttatgatttctgaaattcaatggttctattgactttttgcgtcacttaatccaaatcaaccaaattcagggttgaatctgaattggcttctcaggctcaaggtaacagtgttcttgtggtttgaccaattgtttttctttctttttttttttttttagatttgtggtattctggtcaagttattgtgctgtactttgtgcgtagaaattgagttgtattgtcaaccccagtcagtaaagagaacttcaaaaaattatcctcaagtgtagatttctcttaattccatttgtgtatcatgttaaactattgttgtggcttcttgtgtaaagacaggaactgtggaactgtgatgttgtcttttgtgttgttaaaataagaaatgtcttatctgtatatgtatgagtcttcctgtcattgtatttggcacatgaatattgtgtacaaggaattgttaagactggttttccctcaacaacatatattatacttgctactggaaaagtgtttaagacttagctaggtttccatttagatcttcatatctgttgcatggaagaaagttgggttcttggcatagagttgcatgatatgtaagattttgtgcattcataattgttaaaaatctgtgttccaaaagtggacatagcatgtacaggcagttttctgtcctgtgcacaaaaagtttaaaaaagttgtttaatatttgttgttgtatacccaaatacgcaccgaataaactctttatattcattcaaagaaaaaaaaaaaaaaaaaaaaaaaaaaaaaa

SEQ ID NO: 7 - protein, 199 aa, Homo sapiens
MSGKVTKPKEEKDASKEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 8 - DNA, 2172 bp, Homo sapiens
gtggctctctaggaccggagagttctttggaaggagagcgcgagcgagggagcgggcgagctccgagggggtgtgggtgtagggagagagagaaagagagcaggcagcggcggcggcggcagcggtggggaaaagcggattccgccccgaaccacaccgaggggagctcgtggtcgagacttgccgccctaagcactctcccaagtccgacccgctcggcgaggacttccgtcttctgagcgaaccttgtcaagcaagctgggatctatgagtggaaaggtgaccaagcccaaagaggagaaagatgcttctaaggagcctcagcttaagggtatagttaccaagctatacagccgacaaggctaccacttgcagctgcaggcggatggaaccattgatggcaccaaagatgaggacagcacttacactctgtttaacctcatccctgtgggtctgcgagtggtggctatccaaggagttcaaaccaagctgtacttggcaatgaacagtgagggatacttgtacacctcggaacttttcacacctgagtgcaaattcaaagaatcagtgtttgaaaattattatgtgacatattcatcaatgatataccgtcagcagcagtcaggccgagggtggtatctgggtctgaacaaagaaggagagatcatgaaaggcaaccatgtgaagaagaacaagcctgcagctcattttctgcctaaaccactgaaagtggccatgtacaaggagccatcactgcacgatctcacggagttctcccgatctggaagcgggaccccaaccaagagcagaagtgtctctggcgtgctgaacggaggcaaatccatgagccacaatgaatcaacgtagccagtgagggcaaaagaagggctctgtaacagaaccttacctccaggtgctgttgaattcttctagcagtccttcacccaaaagttcaaatttgtcagtgacatttaccaaacaaacaggcagagttcactattctatctgccattagaccttcttatcatccatactaaagccccattatttagattgagcttgtgcataagaatgccaagcattttagtgaactaaatctgagagaaggactgccaaattttctcatgatctcacctatactttggggatgataatccaaaagtatttcacagcactaatgctgatcaaaatttgctctcccaccaagaaaatgtaaaagaccacaattgttcttcaaaaacaaacaaaacaaaacaaaacaaaattaactgcttaaatgttttgtcggggcaaacaaaattatgtgaattgtgttgttttcttggcttgatgttttctatctacgcttgattcacatgtactcttttctttggcatagtgcaactttatgatttctgaaattcaatggttctattgactttttgcgtcacttaatccaaatcaaccaaattcagggttgaatctgaattggcttctcaggctcaaggtaacagtgttcttgtggtttgaccaattgtttttctttctttttttttttttttagatttgtggtattctggtcaagttattgtgctgtactttgtgcgtagaaattgagttgtattgtcaaccccagtcagtaaagagaacttcaaaaaattatcctcaagtgtagatttctcttaattccatttgtgtatcatgttaaactattgttgtggcttcttgtgtaaagacaggaactgtggaactgtgatgttgtcttttgtgttgttaaaataagaaatgtcttatctgtatatgtatgagtcttcctgtcattgtatttggcacatgaatattgtgtacaaggaattgttaagactggttttccctcaacaacatatattatacttgctactggaaaagtgtttaagacttagctaggtttccatttagatcttcatatctgttgcatggaagaaagttgggttcttggcatagagttgcatgatatgtaagattttgtgcattcataattgttaaaaatctgtgttccaaaagtggacatagcatgtacaggcagttttctgtcctgtgcacaaaaagtttaaaaaagttgtttaatatttgttgttgtatacccaaatacgcaccgaataaactctttatattcattcaaagaaaaaaaaaaaaaaaaaaaaaaaaaaaaaa

SEQ ID NO: 9 - protein, 226 aa, Homo sapiens; identical to SEQ ID NO: 5

SEQ ID NO: 10 - DNA, 2093 bp, Homo sapiens
catgtaacatgtgatttgctcctccttgccttccaccgtgatgtgaggcctccccaaccaagtggaactttctggatgacgccccccctggcacacaggaatacattatgttacgacaagattccatccaatctgcggaattaaagaaaaaagagtccccctttcgtgctaagtgtcacgaaatcttctgctgcccgctgaagcaagtacaccacaaagagaacacagagccggaagagcctcagcttaagggtatagttaccaagctatacagccgacaaggctaccacttgcagctgcaggcggatggaaccattgatggcaccaaagatgaggacagcacttacactctgtttaacctcatccctgtgggtctgcgagtggtggctatccaaggagttcaaaccaagctgtacttggcaatgaacagtgagggatacttgtacacctcggaacttttcacacctgagtgcaaattcaaagaatcagtgtttgaaaattattatgtgacatattcatcaatgatataccgtcagcagcagtcaggccgagggtggtatctgggtctgaacaaagaaggagagatcatgaaaggcaaccatgtgaagaagaacaagcctgcagctcattttctgcctaaaccactgaaagtggccatgtacaaggagccatcactgcacgatctcacggagttctcccgatctggaagcgggaccccaaccaagagcagaagtgtctctggcgtgctgaacggaggcaaatccatgagccacaatgaatcaacgtagccagtgagggcaaaagaagggctctgtaacagaaccttacctccaggtgctgttgaattcttctagcagtccttcacccaaaagttcaaatttgtcagtgacatttaccaaacaaacaggcagagttcactattctatctgccattagaccttcttatcatccatactaaagccccattatttagattgagcttgtgcataagaatgccaagcattttagtgaactaaatctgagagaaggactgccaaattttctcatgatctcacctatactttggggatgataatccaaaagtatttcacagcactaatgctgatcaaaatttgctctcccaccaagaaaatgtaaaagaccacaattgttcttcaaaaacaaacaaaacaaaacaaaacaaaattaactgcttaaatgttttgtcggggcaaacaaaattatgtgaattgtgttgttttcttggcttgatgttttctatctacgcttgattcacatgtactcttttctttggcatagtgcaactttatgatttctgaaattcaatggttctattgactttttgcgtcacttaatccaaatcaaccaaattcagggttgaatctgaattggcttctcaggctcaaggtaacagtgttcttgtggtttgaccaattgtttttctttctttttttttttttttagatttgtggtattctggtcaagttattgtgctgtactttgtgcgtagaaattgagttgtattgtcaaccccagtcagtaaagagaacttcaaaaaattatcctcaagtgtagatttctcttaattccatttgtgtatcatgttaaactattgttgtggcttcttgtgtaaagacaggaactgtggaactgtgatgttgtcttttgtgttgttaaaataagaaatgtcttatctgtatatgtatgagtcttcctgtcattgtatttggcacatgaatattgtgtacaaggaattgttaagactggttttccctcaacaacatatattatacttgctactggaaaagtgtttaagacttagctaggtttccatttagatcttcatatctgttgcatggaagaaagttgggttcttggcatagagttgcatgatatgtaagattttgtgcattcataattgttaaaaatctgtgttccaaaagtggacatagcatgtacaggcagttttctgtcctgtgcacaaaaagtttaaaaaagttgtttaatatttgttgttgtatacccaaatacgcaccgaataaactctttatattcattcaaagaaaaaaaaaaaaaaaaaaaaaaaaaaaaaa

SEQ ID NO: 11 - protein, 192 aa, Homo sapiens
MALLRKSYSEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 12 - DNA, 1968 bp, Homo sapiens
aaactttctctgatctcctctctctctgtgtctgctccaaatgtagacagcaattgtctgggtaggaccagcttataaagaagcatggctttgttaaggaagtcgtattcagagcctcagcttaagggtatagttaccaagctatacagccgacaaggctaccacttgcagctgcaggcggatggaaccattgatggcaccaaagatgaggacagcacttacactctgtttaacctcatccctgtgggtctgcgagtggtggctatccaaggagttcaaaccaagctgtacttggcaatgaacagtgagggatacttgtacacctcggaacttttcacacctgagtgcaaattcaaagaatcagtgtttgaaaattattatgtgacatattcatcaatgatataccgtcagcagcagtcaggccgagggtggtatctgggtctgaacaaagaaggagagatcatgaaaggcaaccatgtgaagaagaacaagcctgcagctcattttctgcctaaaccactgaaagtggccatgtacaaggagccatcactgcacgatctcacggagttctcccgatctggaagcgggaccccaaccaagagcagaagtgtctctggcgtgctgaacggaggcaaatccatgagccacaatgaatcaacgtagccagtgagggcaaaagaagggctctgtaacagaaccttacctccaggtgctgttgaattcttctagcagtccttcacccaaaagttcaaatttgtcagtgacatttaccaaacaaacaggcagagttcactattctatctgccattagaccttcttatcatccatactaaagccccattatttagattgagcttgtgcataagaatgccaagcattttagtgaactaaatctgagagaaggactgccaaattttctcatgatctcacctatactttggggatgataatccaaaagtatttcacagcactaatgctgatcaaaatttgctctcccaccaagaaaatgtaaaagaccacaattgttcttcaaaaacaaacaaaacaaaacaaaacaaaattaactgcttaaatgttttgtcggggcaaacaaaattatgtgaattgtgttgttttcttggcttgatgttttctatctacgcttgattcacatgtactcttttctttggcatagtgcaactttatgatttctgaaattcaatggttctattgactttttgcgtcacttaatccaaatcaaccaaattcagggttgaatctgaattggcttctcaggctcaaggtaacagtgttcttgtggtttgaccaattgtttttctttctttttttttttttttagatttgtggtattctggtcaagttattgtgctgtactttgtgcgtagaaattgagttgtattgtcaaccccagtcagtaaagagaacttcaaaaaattatcctcaagtgtagatttctcttaattccatttgtgtatcatgttaaactattgttgtggcttcttgtgtaaagacaggaactgtggaactgtgatgttgtcttttgtgttgttaaaataagaaatgtcttatctgtatatgtatgagtcttcctgtcattgtatttggcacatgaatattgtgtacaaggaattgttaagactggttttccctcaacaacatatattatacttgctactggaaaagtgtttaagacttagctaggtttccatttagatcttcatatctgttgcatggaagaaagttgggttcttggcatagagttgcatgatatgtaagattttgtgcattcataattgttaaaaatctgtgttccaaaagtggacatagcatgtacaggcagttttctgtcctgtgcacaaaaagtttaaaaaagttgtttaatatttgttgttgtatacccaaatacgcaccgaataaactctttatattcattcaaagaaaaaaaaaaaaaaaaaaaaaaaaaaaaaa

SEQ ID NO: 13 - DNA, 19 bp, Artificial Sequence
  Description of Artificial Sequence Synthetic oligonucleotide
gaacaaagaaggagagatc

SEQ ID NO: 14 - DNA, 19 bp, Artificial Sequence
  Description of Artificial Sequence Synthetic oligonucleotide
cagcttaagggtatagtta

SEQ ID NO: 15 - DNA, 19 bp, Artificial Sequence
  Description of Artificial Sequence Synthetic oligonucleotide
acaaagaaggagagatcat

SEQ ID NO: 16 - DNA, 19 bp, Artificial Sequence
  Description of Artificial Sequence Synthetic oligonucleotide
gcaaccatgtgaagaagaa

SEQ ID NO: 17 - DNA, 19 bp, Artificial Sequence
  Description of Artificial Sequence Synthetic oligonucleotide
gaacaagcctgcagctcat

SEQ ID NO: 18 - DNA, 19 bp, Artificial Sequence
  Description of Artificial Sequence Synthetic oligonucleotide
gcacttacactctgtttaa

SEQ ID NO: 19 - DNA, 19 bp, Artificial Sequence
  Description of Artificial Sequence Synthetic oligonucleotide
gagagatcatgaaaggcaa

SEQ ID NO: 20 - DNA, 19 bp, Artificial Sequence
  Description of Artificial Sequence Synthetic oligonucleotide
tgaaagtggccatgtacaa

SEQ ID NO: 21 - DNA, 24 bp, Artificial Sequence
  Description of Artificial Sequence Synthetic oligonucleotide
caccaccaccgcttcttttgctgc

SEQ ID NO: 22 - DNA, 25 bp, Artificial Sequence
  Description of Artificial Sequence Synthetic primer
tggcattacaagagttagcttctga

SEQ ID NO: 23 - DNA, 25 bp, Artificial Sequence
  Description of Artificial Sequence Synthetic primer
aatgctttgtagtggctttgtttcc

SEQ ID NO: 24 - DNA, 25 bp, Artificial Sequence
  Description of Artificial Sequence Synthetic primer
tggcattacaagagttagcttctga

SEQ ID NO: 25 - DNA, 21 bp, Artificial Sequence
  Description of Artificial Sequence Synthetic primer
cctccagggtgactaaatttg

SEQ ID NO: 26 - DNA, 20 bp, Artificial Sequence
  Description of Artificial Sequence Synthetic primer
aactagaaggccattggctg

SEQ ID NO: 27 - DNA, 25 bp, Artificial Sequence
  Description of Artificial Sequence Synthetic primer
aatgctttgtagtggctttgtttcc

SEQ ID NO: 28 - DNA, 19 bp, Artificial Sequence
  Description of Artificial Sequence Synthetic primer
cagccgacaaggctaccac

SEQ ID NO: 29 - DNA, 22 bp, Artificial Sequence
  Description of Artificial Sequence Synthetic primer
gttccgaggtgtacaagtatcc

SEQ ID NO: 30 - DNA, 20 bp, Artificial Sequence
  Description of Artificial Sequence Synthetic primer
gcagcaggaacttttgacag

SEQ ID NO: 31 - DNA, 20 bp, Artificial Sequence
  Description of Artificial Sequence Synthetic primer
gctggtgtgttccaattcag

SEQ ID NO: 32 - DNA, 20 bp, Artificial Sequence
  Description of Artificial Sequence Synthetic primer
gttgggacgccttgtttagc

SEQ ID NO: 33 - DNA, 23 bp, Artificial Sequence
  Description of Artificial Sequence Synthetic primer
tagcgcgaagaaatatcaaacag

SEQ ID NO: 34 - DNA, 21 bp, Artificial Sequence
  Description of Artificial Sequence Synthetic primer
gcattctgtggaggctctatc

SEQ ID NO: 35 - DNA, 20 bp, Artificial Sequence
  Description of Artificial Sequence Synthetic primer
gctgcattgtagttgtggtg

SEQ ID NO: 36 - DNA, 21 bp, Artificial Sequence
  Description of Artificial Sequence Synthetic primer
ggacatttctggctgcctttg

SEQ ID NO: 37 - DNA, 19 bp, Artificial Sequence
  Description of Artificial Sequence Synthetic primer
ccagaatcgggtatccaag

SEQ ID NO: 38 - DNA, 20 bp, Artificial Sequence
  Description of Artificial Sequence Synthetic primer
gggatcttccagtgggatct

SEQ ID NO: 39 - DNA, 22 bp, Artificial Sequence
  Description of Artificial Sequence Synthetic primer
gcagtcatccagagatgtgacc

SEQ ID NO: 40 - DNA, 20 bp, Artificial Sequence
  Description of Artificial Sequence Synthetic primer
atggacacgctcccctgact

SEQ ID NO: 41 - DNA, 20 bp, Artificial Sequence
  Description of Artificial Sequence Synthetic primer
gaaaggtgggagcctcagtc

SEQ ID NO: 42 - DNA, 19 bp, Artificial Sequence
  Description of Artificial Sequence Synthetic primer
cgagaaatccaacgcctgc

SEQ ID NO: 43 - DNA, 20 bp, Artificial Sequence
  Description of Artificial Sequence Synthetic primer
caccactcgcagacccacag

SEQ ID NO: 44 - DNA, 23 bp, Artificial Sequence
  Description of Artificial Sequence Synthetic primer
gttaaggaagtcgtattcagagc

SEQ ID NO: 45 - DNA, 20 bp, Artificial Sequence
  Description of Artificial Sequence Synthetic primer
caccactcgcagacccacag

SEQ ID NO: 46 - DNA, 21 bp, Artificial Sequence
  Description of Artificial Sequence Synthetic primer
gatgcttctaaggagcctcag

SEQ ID NO: 47 - DNA, 20 bp, Artificial Sequence
  Description of Artificial Sequence Synthetic primer
caccactcgcagacccacag

SEQ ID NO: 48 - DNA, 21 bp, Artificial Sequence
  Description of Artificial Sequence Synthetic primer
acagagccggaagagcctcag

SEQ ID NO: 49 - DNA, 20 bp, Artificial Sequence
  Description of Artificial Sequence Synthetic primer
caccactcgcagacccacag

SEQ ID NO: 50 - DNA, 21 bp, Artificial Sequence
  Description of Artificial Sequence Synthetic primer
gatgcttctaaggttctggat

SEQ ID NO: 51 - DNA, 20 bp, Artificial Sequence
  Description of Artificial Sequence Synthetic primer
caccactcgcagacccacag

SEQ ID NO: 52 - DNA, 21 bp, Artificial Sequence
  Description of Artificial Sequence Synthetic primer
tcaaaccaagctgtatttggc

SEQ ID NO: 53 - DNA, 21 bp, Artificial Sequence
  Description of Artificial Sequence Synthetic primer
ctttcagtggtttgggcagaa

SEQ ID NO: 54 - DNA, 21 bp, Artificial Sequence
  Description of Artificial Sequence Synthetic primer
agcctcagcttaagggtatag

SEQ ID NO: 55 - DNA, 22 bp, Artificial Sequence
  Description of Artificial Sequence Synthetic primer
caagaacactgttaccttgagc

(19) United States
(12) Patent Application Publication
(10) Pub. No.: US 2015/0071934 A1
(43) Pub. Date: Mar. 12, 2015

(54) Methods For Regulating Hair Growth Disorders

(71) Applicant: The Trustees of Columbia University in the City of New York, New York, NY (US)

(72) Inventor: Angela Christiano, Mahwah, NJ (US)

(73) Assignee: The Trustees of Columbia University in the City of New York, New York, NY (US)

(21) Appl. No.: 14/494,239

(22) Filed: Sep. 23, 2014

Related U.S. Application Data
(63) Continuation of application No. PCT/US2013/034683, filed on Mar. 29, 2013.
(60) Provisional application No. 61/617,363, filed on Mar. 29, 2012.

(51) Int. Cl. C07K 16/22; G01N 33/68; C12Q 1/68; C12N 15/113

(52) U.S. Cl. CPC C07K 16/22; C12N 15/1136; G01N 33/6893; C12Q 1/6883; C12N 2310/111; C07K 2317/76; G01N 2333/50; C12Q 2600/158
USPC 424/139.1; 514/44 A; 436/501; 435/6.12

(57) ABSTRACT

The invention provides for methods for treating a hair loss disorder in a subject by administering a FGF13 inhibitor. The invention further provides for methods for treating a hair growth disorder in a subject by administering a FGF13 activator.

This application claims the benefit of and priority to International Application No. PCT/U.S. Ser. No. 13/034,683, filed Mar. 29, 2013 and U.S. Provisional Patent Application No. 61/617,363, filed on Mar. 29, 2012, the content of each which are hereby incorporated by reference in their entireties.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Alopecia Areata (AA) is one of the most highly prevalent autoimmune diseases, leading to hair loss due to the collapse of immune privilege of the hair follicle and subsequent autoimmune destruction. AA is a skin disease which leads to hair loss on the scalp and elsewhere. In some severe cases, it can progress to complete loss of hair on the head or body. Although Alopecia Areata is believed to be caused by autoimmunity, the gene level diagnosis and treatment are seldom reported. The genetic basis of AA is largely unknown.

Hypertrichosis is defined as excessive hair growth for a particular site of the body or age of a patient that is not hormone-dependent. Hypertrichoses are characterized on the basis of multiple criteria: cause (genetic or acquired), age of onset, extent of hair distribution (universal or localized) and affected sites. We are studying several different forms of hypertrichosis in humans, including X-linked hypertrichosis (OMIM 307150), generalized hypertrichosis terminalis with or without gingival hyperplasia (CGHT; OMIM 135400), autosomal recessive hypertrichosis, Cantu syndrome (OMIM 239850), Ambras type hypertrichosis (AS; OMIM 145701) and autosomal recessive trichomegaly (OMIM 190330).

### SUMMARY OF THE INVENTION

An aspect of the invention encompasses a method of treating a hair-loss disorder in a mammalian subject in need thereof, the method comprising administering to the subject an inhibitor of FGF1. In one embodiment, the hair-loss disorder comprises androgenetic alopecia, telogen effluvium, alopecia areata, tinea capitis, alopecia totalis, hypotrichosis, hereditary hypotrichosis simplex, or alopecia universalis. In one embodiment, the method further comprises the step (b) determining whether the inhibitor administered induced hair growth in the subject afflicted with a hair loss disorder as compared to the subject's hair growth prior to treatment with the inhibitor. In one embodiment, the inhibitor comprises an antibody that specifically binds to a protein comprising SEQ ID NO: 1, 3, 5, 7, 9, or 1. In another embodiment, the inhibitor is an antisense RNA that specifically inhibits expression of the gene that encodes the FGF13 protein; a siRNA that specifically targets the gene that encodes the FGF13 protein, or a small molecule. In one embodiment, the siRNA is directed to a human nucleic acid sequence comprising SEQ ID NO: 2, 4, 6, 8, 10, or 1. In another embodiment, the siRNA directed to a FGF13 gene is any one of the sequences listed in Table 1.

Another aspect of the invention encompasses a method for inducing hair growth in a subject, the method comprising administering to the subject an effective amount of an inhibitor of FGF13, thereby controlling hair growth in the subject. In one embodiment, the subject is afflicted with a hair-loss disorder. In another embodiment, the hair-loss disorder comprises androgenetic alopecia, telogen effluvium, alopecia areata, tinea capitis, alopecia totalis, hypotrichosis, hereditary hypotrichosis simplex, or alopecia universalis. In one embodiment, the method further comprises the step (b) determining whether the inhibitor administered induced hair growth in the subject afflicted with a hair loss disorder as compared to the subject's hair growth prior to treatment with the inhibitor. In one embodiment, the inhibitor comprises an antibody that specifically binds to a protein comprising SEQ ID NO: 1, 3, 5, 7, 9, or 1. In another embodiment, the inhibitor is an antisense RNA that specifically inhibits expression of the gene that encodes the FGF13 protein; a siRNA that specifically targets the gene that encodes the FGF13 protein, or a small molecule. In one embodiment, the siRNA is directed to a human nucleic acid sequence comprising SEQ ID NO: 2, 4, 6, 8, 10, or 1. In another embodiment, the siRNA directed to a FGF13 gene is any one of the sequences listed in Table 1.

Another aspect of the invention encompasses a method of treating a hair-growth disorder in a mammalian subject in need thereof, the method comprising administering to the subject an activator of FGF1. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and autosomal recessive trichomegaly. In one embodiment, the activator is a polypeptide comprising SEQ ID NO: 1, 3, 5, 7, 9, or 11, or a fragment thereof; or a peptidomimetic comprising SEQ ID NO: 1, 3, 5, 7, 9, or 11.

Another aspect of the invention encompasses a method for reducing hair growth in a subject, the method comprising administering to the subject an effective amount of an activator of FGF13, thereby controlling hair growth in the subject. In one embodiment, the subject is afflicted with a hair-growth disorder. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and autosomal recessive trichomegaly. In another embodiment, the method further comprises the step (b) determining whether the activator administered reduced hair growth in the subject afflicted with a hair-growth disorder as compared to the subject's hair growth prior to treatment with the activator. In one embodiment, the activator is a polypeptide comprising SEQ ID NO: 1, 3, 5, 7, 9, or 11, or a fragment thereof; or a peptidomimetic comprising SEQ ID NO: 1, 3, 5, 7, 9, or 11.

Another aspect of the invention encompasses a method for reducing hair growth in a subject, the method comprising administering to the subject an effective amount of a FGF13 protein, thereby controlling hair growth in the subject. In one embodiment, the subject is afflicted with a hair-growth disorder. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and autosomal recessive trichomegaly. In another embodiment, the method further comprises the step (b) determining whether the FGF13 protein administered reduced hair growth in the subject afflicted with a hair-growth disorder as compared to the subject's hair growth prior to treatment with the protein.

Another aspect of the invention encompasses a method of treating a hair disorder in a mammalian subject in need thereof, the method comprising administering to the subject a compound that modulates the expression of FGF1. In one embodiment, the hair disorder is a hair-loss disorder. In one embodiment, the hair-loss disorder comprises androgenetic alopecia, telogen effluvium, alopecia areata, tinea capitis, alopecia totalis, hypotrichosis, hereditary hypotrichosis simplex, or alopecia universalis. In another embodiment, the hair disorder is a hair-growth disorder. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and autosomal recessive trichomegaly. In one embodiment, the method further comprises the step (b) determining whether the compound administered induced hair growth in the subject afflicted with a hair loss disorder as compared to the subject's hair growth prior to treatment with the compound. In another embodiment, the method further comprises the step (b) determining whether the compound administered reduced hair growth in the subject afflicted with a hair-growth disorder as compared to the subject's hair growth prior to treatment with the compound.

In a further embodiment, the administering comprises a subcutaneous, intra-muscular, intra-peritoneal, or intravenous injection; an infusion; oral, nasal, or topical delivery; or a combination thereof. In some embodiments, the administering occurs daily, weekly, twice weekly, monthly, twice monthly, or yearly.

An aspect of the invention provides for a method for determining the presence of or a predisposition to developing a hair-growth disorder in a subject. The method comprises extracting a sample from a subject, and detecting the presence, absence, or reduction of an FGF13 protein in the subject as compared to a subject not afflicted with a hair growth disorder, wherein the absence or reduction of the FGF13 protein is indicative of a hair growth disorder. In one embodiment, the method further comprises incubating the sample with an agent that binds an FGFG13 protein, or fragment thereof. In another embodiment, the agent is an antibody that specifically binds to the FGF13 protein, or fragment thereof. In a further embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis, autosomal recessive trichomegaly or a combination thereof.

An aspect of the invention provides for a method for determining the presence of or a predisposition to developing a hair-growth disorder in a subject. The method comprises extracting a sample from a subject, and detecting the presence, absence, or reduction of an FGF13 nucleic acid in the subject as compared to a subject not afflicted with a hair growth disorder, wherein the reduction of the FGF13 nucleic acid is indicative of a hair growth disorder. In one embodiment, the detecting comprises using PCR and primers directed to SEQ ID NO: 2, 4, 6, 8, 10, or 1. In another embodiment, the primer comprises SEQ ID NO: 24, 25, 26, 27, 28, 29, 54, 5. In a farther embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis, autosomal recessive trichomegaly or a combination thereof.

### BRIEF DESCRIPTION OF THE FIGURES

FIG. 1 are photographic images of clinical manifestations of AA. In the upper panels (FIGS. 1A-B), patients with AA multiplex. In FIG. 1B, the patient is in regrowth phase. For patients with alopecia universalis, there is a complete lack of body hair and scalp hair (FIG. 1C), while patients with alopecia totalis only lack scalp hair (FIG. 1D). In FIG. 1D, hair regrowth is observed in the parietal region, while no regrowth in either occipital or temporal regions is evident.

FIG. 2 shows a schematic diagram showing an inherited 389 kb interchromosomal insertion at Xq27.1 in a Mexican family with CGH. The insertion contains a 389 kb segment of chromosome 6p21.2 in reverse orientation, as well as a 56 bp segment of chromosome 3q21.1 in reverse orientation, separated by 14 bp of unknown origin. The insertion also contains a 6 bp sequence of unknown origin at the centromeric breakpoint, and additionally results in a 2 bp deletion at this junction.

FIG. 3 shows that the relative quantity of FGF13 in the cDNA from normal control scalp (S113), two carrier individuals (125-04 and 125-17) and three CGH affected individuals (125-05, 125-27, and 125-28) was calculated on an ABI 7300 quantitative PCR machine using the ABI Relative Quantification Study software. The amount of FGF13 was standardized to the housekeeping gene B2M and calculated as relative to the unaffected scalp sample.

FIG. 4 shows pedigree (FIG. 4A) and clinical features (FIG. 4B) of a Mexican family with congenital universal hypertrichosis, deafness and dental anomalies. Clinical pictures of affected males with (a) excessive hair growth on the back, shoulders and arms, (b) dental malformations and thickened lips, (c) excessive hair growth on the chest and shoulders and (d) extreme hair overgrowth on face, dental anomalies and bulbous nose are shown.

FIG. 5 is a photomicrograph of immunofluorescence staining of skin biopsies from control (FIG. SA) and affected (FIG. 5B) individuals using an anti-SOX9 antibody

FIG. 6 is a photomicrograph of immunofluorescence staining of a normal human scalp frozen biopsy using an anti-USP53 antibody.

FIGS. 7A-B show pedigrees and clinical phenotype of hypertrichosis families. Two non-consanguineous families from Pakistan presenting with autosomal recessive trichomegaly. For simplicity, the pedigree of family HYPR1 (FIG. 7A) was broken down into 5 consanguineous subfamilies. (C) Consanguineous family from Pakistan showing an autosomal recessive hypertrichosis phenotype.

FIG. 7C shows pedigrees and clinical phenotype of hypertrichosis Consanguineous family from Pakistan showing an autosomal recessive hypertrichosis phenotype

FIG. 8 shows a pedigree and clinical phenotype of a Pakistani family with gingival hyperplasia.

FIG. 9 depicts X-linked recessive hypertrichosis with linkage on Xq24-27 in a Mexican family. The bottom shows a pedigree of a four-generation family, three of whom are obligate carriers and eight of whom are affected.

FIG. 10 depicts X-linked recessive hypertrichosis with linkage on Xq24-27. Fluorescent in situ hybridization (FISH) of probes to chromosome 6 and X demonstrates the insertion at Xq27.

FIG. 11 depicts X-linked recessive hypertrichosis with linkage on Xq24-27.

FIG. 12 shows that FGF13 is down-regulated in X-linked CGH patients.

FIG. 13 shows that FGF13 is down-regulated in X-linked CGH patients. PCR using human fetal brain cDNA and scalp cDNA revealed that the other genes in the interchromosomal insertion region are not expressed in skin.

FIG. 14 shows that FGF13 is expressed in the E13.5 and E14.5 whisker pad.

FIG. 15 is a bar graph showing FGF13 expression in E12.5-16.5 epidermis and dermis.

FIG. 16 are photomicrographic images of FGF13 expression in the E14.5 epidermis, dermis, and whisker pad. Images on left were taken from the craniofacial area.

FIG. 17 are photomicrographs showing immunohistochemical detection of Fgf13 in the developing pelage follicles. See Woo and Oro (2011) Cell, 146(2):334-334.e2.

FIG. 18 are photomicrographs showing Fgf13 localization to the bulge and basal layer of the epidermis in mouse anagen hair follicles (HFs).

FIG. 19 are photomicrographs showing in situ hybridization of FGF13 in the human HF.

FIG. 20 are photomicrographs showing immunofluorescent staining of FGF13 in the human HF.

FIGS. 21A-B are photographs of the clinical features of hair follicles in a Mexican family with X-linked CGH, deafness, palate and dental anomalies. Clinical photos of affected males with excessive hair growth on the back, shoulders, arms (A, B).

FIG. 22 is a photograph showing the clinical features of hair follicles in a Mexican family with X-linked CGH, deafness, palate and dental anomalies. Moderate hair growth is observed on the back of a female carrier; the inset represents a close-up image of a cowlick on the back of another carrier.

FIG. 23 shows photomicrographs of histology of a normal hair follicle and affected hair follicle, both from males, revealed that the hairs are of the terminal type, as they are medullated and highly pigmented. (A, B) Affected hair follicles have a thickened inner root sheath (IRS). (C, D) End bulbs of the control and affected hair follicles, where a widened dermal papilla (white arrows), matrix (Mx), and hair shaft (HS) is observed in affected hair follicles. Scale bars=100 μm.

FIG. 24A shows an SNP oligonucleotide microarray analysis (SOMA) that revealed a 386 kb duplication of chromosome 6 and FISH confirmed its insertion on the X chromosome. SOMA performed on an affected individual using the Affymetrix Cytogenetics Whole-Genome 2.7M array revealed a 386 kb duplication of chromosome 6p21.2 encompassing the KIF6 and DAAM2 genes (as shown in FIG. 24C).

FIG. 24B shows an SNP oligonucleotide microarray analysis (SOMA) that revealed a 386 kb duplication of chromosome 6 and FISH confirmed its insertion on the X chromosome. FISH on control and carrier metaphase chromosomes confirmed the insertion of the chromosome 6 duplication onto the X chromosome at the cytogenetic level, where boxes indicate X chromosomes, white arrows indicate chromosome 6, and the red arrow indicates the X chromosome containing the insertion. Insets are magnified images of the unaffected and affected X chromosome from control and carrier individuals, respectively.

FIG. 24C shows an SNP oligonucleotide microarray analysis (SOMA) that revealed a 386 kb duplication of chromosome 6 and FISH confirmed its insertion on the X chromosome. Non-overlapping BAC clones used to span the chromosome 6 duplication include the KIF6 and DAAM2 genes (drawn to scale). Genomic coordinates reference UCSC human reference genome build hg19.

FIGS. 25A-B shows schematics of whole-genome sequencing that revealed a 389 kb interchromosomal insertion at Xq27.1 that co-segregates with the X-linked hypertrichosis phenotype. (A) Chromosome Xq27.1 in X-linked hypertrichosis. The genes and miRNAs encoded in the surrounding region are shown as black boxes with arrows indicating the direction of transcription. (B) WGS was used to determine the breakpoints and content of the interchromosomal insertion (shown in blue), including the 386 kb duplication from chromosome 6p21.2, 14 bp of unknown origin, and 56 bp of chromosome 3q21.2.

FIG. 25C shows whole-genome sequencing that revealed a 389 kb interchromosomal insertion at Xq27.1 that co-segregates with the X-linked hypertrichosis phenotype. PCR amplification of the centromeric and telomeric junctions of the insertion on DNA from control, carrier, and affected individuals demonstrated segregation of the X-linked phenotype in the family at the genomic level. CB, TB, and C represent centromeric breakpoint, telomeric breakpoint, and controls, respectively. M=marker (1 kb+ ladder).

FIG. 25D shows whole-genome sequencing that revealed a 389 kb interchromosomal insertion at Xq27.1 that co-segregates with the X-linked hypertrichosis phenotype. Primer design of the centromeric and telomeric junctions, where colored arrows correspond to the amplicons produced as shown in FIG. 25C. All images are drawn to scale. Genomic coordinates reference UCSC human reference genome build hg19.

FIGS. 26A-C show that FGF13 levels are reduced in X-linked hypertrichosis and FGF13 is expressed in the human hair follicle. (A) Quantitative RT-PCR of candidate genes surrounding the insertion on control, carrier, and affected skin biopsies reveals that FGF13 levels are reduced by approximately 4-fold in affected individuals relative to controls. (B) FGF13 qRT-PCR in control, carrier, and affected individuals reveals a dosage effect. (C) FGF13 expression normalized to K14 reveals that FGF13 levels are dramatically reduced by approximately 18-fold in affected cells relative to that of controls. Quantitative RT-PCR results are representative of the averaged values of three independent experiments on three controls, three carriers and three affected individuals. Values are relative to the unaffected samples and standardized to the housekeeping gene GAPDH (in A-C). A student's t-test was performed comparing each value to control 1 with a cut-off p value of 0.05 for statistical significance; *=p<0.05; **=p<0.01; ***=p<0.001. Error bars represent the standard error of the mean.

FIGS. 26D-F show that FGF13 levels are reduced in X-linked hypertrichosis and FGF13 is expressed in the human hair follicle. (D) In situ hybridization of FGF13 in anagen hair follicles reveals expression in the outer root sheath (ORS) within the middle and upper portions of the hair follicle, where the sense probe produced no signal. (E) Immunofluorescence staining reveals that FGF13 localizes to the outer root sheath (magnified image) within the middle and upper portions of the human anagen hair follicle (n=5). ORS=outer root sheath; IRS=inner root sheath; HS=hair shaft. (F) FGF13 expression is detected in the trichilemma (outer root sheath) of telogen club hair follicles by immunofluorescence staining. Scale bar indicates 100

FIGS. 27A-B show immunofluorescence staining that reveals that FGF13 expression is dramatically reduced in affected hair follicles compared to control. (A) Immunofluorescence staining in control and affected anagen hair follicles reveals a decrease in FGF13 localization throughout the outer root sheath (ORS) in the mid- and upper portions of the hair follicle. (B) Immunofluorescence staining in carrier and affected telogen hair follicles reveals decreased FGF13 expression in the affected hair follicle, recapitulating the dosage effect seen at the mRNA level. Z-stack images were taken using identical settings and a consistent Z-stack interval between control, carrier, and affected samples. ORS=outer root sheath, CH=club hair of a telogen follicle. Scale bar indicates 100

FIGS. 27C-D show immunofluorescence staining that reveals that FGF13 expression is dramatically reduced in affected hair follicles compared to control. (C) Quantification of the percent FGF13-expressing outer root sheath cells in control and affected hair follicles reveals a decrease in the number of FGF13-expressing cells within the upper and mid-follicle regions of the outer root sheath (p<0.05). Data represent the averaged value of three independent experiments, where images taken at a 40× magnification were used to quantify the number of FGF13-positive cells relative to the total number of outer root sheath cells. For immunofluorescence studies, hair follicles were stained from three control and two affected skin biopsies. (D) Quantitative RT-PCR revealed that FGF13 expression is reduced in keratinocytes but not fibroblasts grown from skin biopsies. A student's t-test was performed with a cut-off p value of 0.05 for statistical significance; *=p<0.05. Error bars represent the standard error of the mean.

FIG. 28 shows morphometric analysis of patient hair follicles reveals matrix, dermal papilla, and hair shaft defects. Quantification of hair follicle components using the length-measuring tool in the AxioVision program revealed a widened dermal papilla (3-fold increase; p=0.0000343), matrix (1.9-fold increase; p=0.0000642), and hair shaft (1.25-fold increase; p=0.036). Inner root sheath (IRS) differences in the lower and upper ORS were not statistically significant. Hair follicles were analyzed from one control and two affected individuals, where the average widest distance was calculated for each hair follicle structure. A student's t-test was performed comparing the affected to control value with a cut-off p value of 0.05 for statistical significance; *=p<0.05; ***=p<0.001. Error bars represent the standard deviation.

FIG. 29 shows a summary of interchromosomal insertion events in all three X-linked CGH families. Zoomed out view of chromosome Xq27.1 region in X-linked hypertrichosis, where boxes indicate the insertion events and sizes of each from chromosomes 4, 5, and 6. Colored lines connected to the inverted triangle indicate orientation of the insertion events, where all three occur at the same palindromic sequence at Xq27.1. The superscript numbers correspond to references.

FIG. 30 are photomicrographs showing that FGF13 localizes to all layers of the ORS and the companion layer but not the human hair follicle bulge. (A) Immunofluorescence staining of FGF13 juxtaposed with KRT14 (which marks all layers of the ORS) demonstrates that FGF13 is broadly expressed throughout the ORS. The far right image is a hematoxylin and eosin staining of an anagen hair follicle for reference of morphology. (B) Co-staining of FGF13 with KRT75, a marker of the companion layer between the ORS and IRS demonstrates that FGF13 localizes to the companion layer (arrows). (C) Co-staining of FGF13 and CD200 (bulge marker) demonstrates that FGF13 does not localize to the human hair follicle bulge. ORS=outer root sheath; IRS=inner root sheath; BL basal layer. Scale bar indicates 100 μm.

FIG. 31 are photomicrographs showing that Fgf13 is expressed in the developing and cycling mouse hair follicle. (A) Whole-mount and section in situ hybridization of Fgf13 on E14.5 embryos reveals expression in the developing whisker pad (WP) and guard hair follicle placodes and dermal condensates at E14.5 (n=3; AS=antisense; S=sense). Arrows indicate vibrissa and guard hair follicles. (B) Immunofluorescence staining on E16.5 vibrissae follicles demonstrates that Fe13 localizes to the outer root sheath (ORS) (n=3). (C) Immunofluorescence staining of Fe13 in anagen (day 30) hair follicles reveals that Fgf13 localizes to the bulge (B), isthmus (I), and outer root sheath (ORS). (D) Co-staining of Fgf13 and CD200 in telogen (day 50) hair follicles reveals that Fgf13 localizes to the bulge. Scale bar indicates 100 μm.

FIG. 32A shows isoform-specific PCR of FGF13 in whole skin. Schematic of the FGF13 locus at Chr.Xq26.3-27.1. Alternating 5′ exons (termed 1S, 1U, 1V, 1 Y, and 1V+1Y) are represented as boxes with distinct colors, whereas exons 2-5 common to all transcripts are shown as blue boxes. The dark box at the 5′ end of the 1S isoform represents a nuclear localization signal. Scale bar represents 100 kb.

FIG. 32B shows isoform-specific PCR of FGF13 in whole skin. Amplification of FGF13 transcripts using eDNA from whole skin demonstrates that the 1S, 1 Y, and 1V+1 Y isoforms are strongly expressed, where the V isoform is faintly expressed. ‘Core’ represents the 3′ region common to all these isoforms. The Ensemb1 transcripts corresponding to these splice variants are: FGF13-001 (1S), FGF13-002 (1U), FGF13-203 (1V), FGF13-202 (1Y), FGF13-201, 3 (1V+Y).

### DETAILED DESCRIPTION OF THE INVENTION

A reduction in the expression in the FGF13 gene has been identified in individuals with excess hair (hypertrichosis). The reduced expression is the result of a position effect/intrachromosomal insertion next to the FGF13 gene. Expression of the FGF13 gene has previously been shown in the hair follicle in both mouse (Kawano et al, Journal of Investigative Dermatology (2004) 122, 1084-10900 and humans (Ohyama et al J Clin Invest. 2006 116:249-60; Oyhama et al J Dermatol Sci. 2007 45:147-50). An intrachromosomal insertion in the region of the X chromosome that causes hair overgrowth has been previously identified (Zhu et al. Am J Hum Genet. 2011 88:819-26).

The present invention provides that the under-expression of FGF13 can be causally linked to the excessive hair growth, thus indicating that pharmacological inhibition of FGF13 can increase hair growth. The invention thus provides for methods of treating a hair loss disorder (e.g., Alopecia Areata (AA), a common autoimmune form of hair loss) with an inhibitor of FGF13. The invention provides for therapeutics previously untested in AA, that can inform one about the clinical relevance of this pathway in AA and related diseases. The invention further provides for methods of treating a hair growth disorder, such as hypertrichosis, with an activator of FGF13.

### ABBREVIATIONS AND DEFINITIONS

The singular forms “a”, “an” and “the” include plural reference unless the context clearly dictates otherwise.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Overview of the Integument and Hair Cells

The integument (or skin) is the largest organ of the body and is a highly complex organ covering the external surface of the body. It merges, at various body openings, with the mucous membranes of the alimentary and other canals. The integument performs a number of essential functions such as maintaining a constant internal environment via regulating body temperature and water loss; excretion by the sweat glands; but predominantly acts as a protective barrier against the action of physical, chemical and biologic agents on deeper tissues. Skin is elastic and except for a few areas such as the soles, palms, and ears, it is loosely attached to the underlying tissue. It also varies in thickness from 0.5 mm (0.02 inches) on the eyelids (“thin skin”) to 4 mm (0.17 inches) or more on the palms and soles (“thick skin”) (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The skin is composed of two layers: a) the epidermis and b) the dermis. The epidermis is the outer layer, which is comparatively thin (0.1 mm). It is several cells thick and is composed of 5 layers: the stratum germinativum, stratum spinosum, stratum granulosum, stratum lucidum (which is limited to thick skin), and the stratum corneum. The outermost epidermal layer (the stratum corneum) consists of dead cells that are constantly shed from the surface and replaced from below by a single, basal layer of cells, called the stratum germinativum. The epidermis is composed predominantly of keratinocytes, which make up over 95% of the cell population. Keratinocytes of the basal layer (stratum germinativum) are constantly dividing, and daughter cells subsequently move upwards and outwards, where they undergo a period of differentiation, and are eventually sloughed off from the surface. The remaining cell population of the epidermis includes dendritic cells such as Langerhans cells and melanocytes. The epidermis is essentially cellular and non-vascular, containing little extracellular matrix except for the layer of collagen and other proteins beneath the basal layer of keratinocytes (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

The dermis is the inner layer of the skin and is composed of a network of collagenous extracellular material, blood vessels, nerves, and elastic fibers. Within the deiinis are hair follicles with their associated sebaceous glands (collectively known as the pilosebaceous unit) and sweat glands. The interface between the epidermis and the dermis is extremely irregular and uneven, except in thin skin. Beneath the basal epidermal cells along the epidermal-dermal interface, the specialized extracellular matrix is organized into a distinct structure called the basement membrane (Ross M H, Histology: A text and atlas, 3^{rd} edition, Williams and Wilkins, 1995: Chapter 14; Burkitt H G, et al, Wheater's Functional Histology, 3′^{d} Edition, Churchill Livingstone, 1996: Chapter 9).

The mammalian hair fiber is composed of keratinized cells and develops from the hair follicle. The hair follicle is a peg of tissue derived from a downgrowth of the epidermis, which lies immediately underneath the skin's surface. The distal part of the hair follicle is in direct continuation with the external, cutaneous epidermis. Although a small structure, the hair follicle comprises a highly organized system of recognizably different layers arranged in concentric series. Active hair follicles extend down through the dermis, the hypodermis (which is a loose layer of connective tissue), and into the fat or adipose layer (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

At the base of an active hair follicle lies the hair bulb. The bulb consists of a body of dermal cells, known as the dermal papilla, contained in an inverted cup of epidermal cells known as the epidermal matrix. Irrespective of follicle type, the germinative epidermal cells at the very base of this epidermal matrix produce the hair fiber, together with several supportive epidermal layers. The lowermost dermal sheath is contiguous with the papilla basal stalk, from where the sheath curves externally around all of the hair matrix epidermal layers as a thin covering of tissue. The lowermost portion of the dermal sheath then continues as a sleeve or tube for the length of the follicle (Ross M H, Histology: A text and atlas, 3^{rd} edition, Williams and Wilkins, 1995: Chapter 14; Burkitt H G, et al, Wheater's Functional Histology, 3^{rd} Edition, Churchill Livingstone, 1996: Chapter 9).

Developing skin appendages, such as hair and feather follicles, rely on the interaction between the epidermis and the dermis, the two layers of the skin. In embryonic development, a sequential exchange of information between these two layers supports a complex series of morphogenetic processes, which results in the formation of adult follicle structures. However, in contrast to general skin dermal and epidermal cells, certain hair follicle cell populations, following maturity, retain their embryonic-type interactive, inductive, and biosynthetic behaviors. These properties can be derived from the very dynamic nature of the cyclical productive follicle, wherein repeated tissue remodeling necessitates a high level of dermal-epidermal interactive communication, which is vital for embryonic development and would be desirable in other forms of tissue reconstruction.

The hair fiber is produced at the base of an active follicle at a very rapid rate. For example, follicles produce hair fibers at a rate 0.4 mm per day in the human scalp and up to 1.5 mm per day in the rat vibrissa or whiskers, which means that cell proliferation in the follicle epidermis ranks amongst the fastest in adult tissues (Malkinson F D and J T Kearn, Int J Dermatol 1978, 17:536-551). Hair grows in cycles. The anagen phase is the growth phase, wherein up to 90% of the hair follicles said to be in anagen; catagen is the involuting or regressing phase which accounts for about 1-2% of the hair follicles; and telogen is the resting or quiescent phase of the cycle, which accounts for about 10-14% of the hair follicles. The cycle's length varies on different parts of the body.

Hair follicle formation and cycling is controlled by a balance of inhibitory and stimulatory signals. The signaling cues are potentiated by growth factors that are members of the TGFβ-BMP family. A prominent antagonist of the members of the TGFβ-BMP family is follistatin. Follistatin is a secreted protein that inhibits the action of various BMPs (such as BMP-2, -4, -7, and -11) and activins by binding to said proteins, and purportedly plays a role in the development of the hair follicle (Nakamura M, et al., FASEB J, 2003, 17(3):497-9; Patel K Intl J Biochem Cell Bio, 1998, 30:1087-93; Ueno N, et al., PNAS, 1987, 84:8282-86; Nakamura T, et al., Nature, 1990, 247:836-8; Iemura S, et al., PNAS, 1998, 77:649-52; Fainsod A, et al., Mech Dev, 1997, 63:39-50; Gamer L W, et al., Dev Biol, 1999, 208:222-32).

The deeply embedded end bulb, where local dermal-epidermal interactions drive active fiber growth, is the signaling center of the hair follicle comprising a cluster of mesenchymal cells, called the dermal papilla (DP). This same region is also central to the tissue remodeling and developmental changes involved in the hair fiber's or appendage's precise alternation between growth and regression phases. The DP, a key player in these activities, appears to orchestrate the complex program of differentiation that characterizes hair fiber formation from the primitive germinative epidermal cell source (Oliver R F, J Soc Cosmet Chem, 1971, 22:741-755; Oliver R F and C A Jahoda, Biology of Wool and Hair (eds Roger et al.), 1971, Cambridge University Press:51-67; Reynolds A J and C A Jahoda, Development, 1992, 115:587-593; Reynolds A J, et al., J Invest Dermatol, 1993, 101:634-38).

The lowermost dermal sheath (DS) arises below the basal stalk of the papilla, from where it curves outwards and upwards. This dermal sheath then externally encases the layers of the epidermal hair matrix as a thin layer of tissue and continues upward for the length of the follicle. The epidermally-derived outer root sheath (ORS) also continues for the length of the follicle, which lies immediately internal to the dermal sheath in between the two layers, and forms a specialized basement membrane termed the glassy membrane. The outer root sheath constitutes little more than an epidermal monolayer in the lower follicle, but becomes increasingly thickened as it approaches the surface. The inner root sheath (IRS) forms a mold for the developing hair shaft. It comprises three parts: the Henley layer, the Huxley layer, and the cuticle, with the cuticle being the innermost portion that touches the hair shaft. The IRS cuticle layer is a single cell thick and is located adjacent to the hair fiber. It closely interdigitates with the hair fiber cuticle layer. The Huxley layer can comprise up to four cell layers. The IRS Henley layer is the single cell layer that runs adjacent to the ORS layer (Ross M H, Histology: A text and atlas, 3^{rd} edition, Williams and Wilkins, 1995: Chapter 14; Burkitt H G, et al, Wheater's Functional Histology, 3¹′^{d} Edition, Churchill Livingstone, 1996: Chapter 9).

### Alopecia Areata

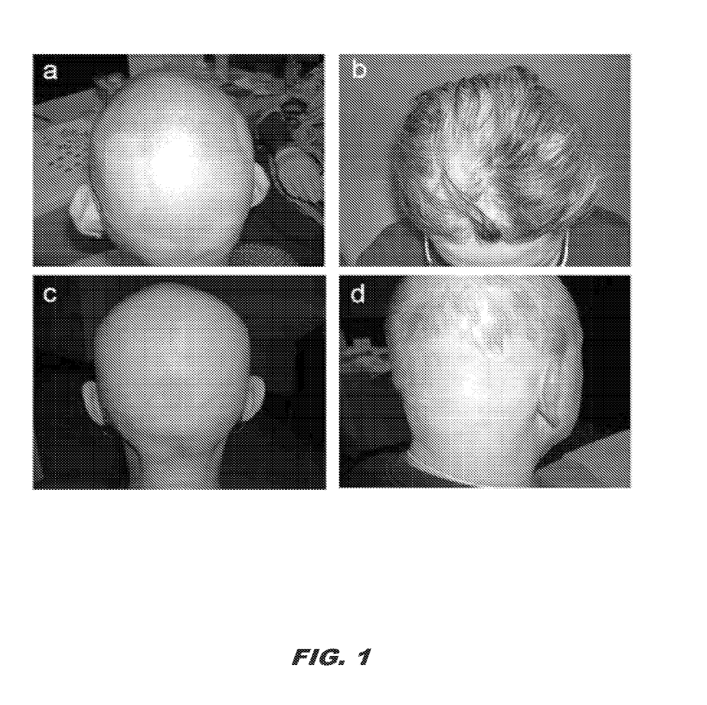

Alopecia areata (AA) is one of the most prevalent autoimmune diseases, affecting approximately 4.6 million people in the US alone, including males and females across all ethnic groups, with a lifetime risk of 1.7% (1) In AA, autoimmunity develops against the hair follicle, resulting in non-scarring hair loss that can begin as patches, which can coalesce and progress to cover the entire scalp (alopecia totalis, AT) or eventually the entire body (alopecia universalis, AU) (FIG. 1). AA was first described by Cornelius Celsus in 30 A.D., using the term “ophiasis”, which means “snake”, due to the sinuous path of hair loss as it spread slowly across the scalp. Hippocrates first used the Greek word ‘alopekia’ (fox mange), the modern day term “alopecia areata” was first used by Sauvages in his Nosologica Medica, published in 1760 in Lyons, France.

Curiously, AA preferentially affects pigmented hair follicles in the anagen (growth) phase of the hair cycle, and when the hair regrows in patches of AA, it frequently grows back white or colorless. The phenomenon of ‘sudden whitening of the hair’ is therefore ascribed to AA with an acute onset, and has been documented throughout history as having affected several prominent individuals at times of profound grief, stress or fear (2). Examples include Shahjahan, who upon the death of his wife in 1631 experienced acute whitening of his hair, and in his grief built the Taj Mahal in her honor. Sir Thomas More, author of Utopia, who on the eve of his execution in 1535 was said to have become ‘white in both beard and hair’. The sudden whitening of the hair is believed to result from an acute attack upon the pigmented hair follicles, leaving behind the white hairs unscathed.

Several clinical aspects of AA remain unexplained but can hold important clues toward understanding pathogenesis. AA attacks hairs only around the base of the hair follicles, which are surrounded by dense clusters of lymphocytes, resulting in the pathognomic ‘swarm of bees’ appearance on histology. Based on these observations, it is postulated that a signal(s) in the pigmented, anagen hair follicle is emitted which invokes an acute or chronic immune response against the lower end of the hair follicle, leading to hair cycle perturbation, acute hair shedding, hair shaft anomalies and hair breakage. Despite these dramatic perturbations in the hair follicle, there is no peunanent organ destruction and the possibility of hair regrowth remains if immune privilege can be restored.

Throughout history, AA has been considered at times to be a neurological disease brought on by stress or anxiety, or as a result of an infectious agent, or even hormonal dysfunction. The concept of a genetically-determined autoimmune mechanism as the basis for AA emerged during the 20^{th} century from multiple lines of evidence. AA hair follicles exhibit an immune infiltrate with activated Th, Tc and NK cells (3, 4) and there is a shift from a suppressive (Th2) to an autoimmune (Th1) cytokine response. The humanized model of AA, which involves transfer of AA patient scalp onto immune-deficient SCID mice illustrates the autoimmune nature of the disease, since transfer of donor T-cells causes hair loss only when co-cultured with hair follicle or human melanoma homogenate (5, 6). Regulatory T cells which serve to maintain immune tolerance are observed in lower numbers in AA tissue (7), and transfer of these cells to C3H/HeJ mice leads to resistance to AA (8). Although AA has long been considered exclusively as a T-cell mediated disease, in recent years, an additional mechanism of disease has been postulated. The hair follicle is defined as one of a select few immune privileged sites in the body, characterized by the presence of extracellular matrix barriers to impede immune cell trafficking, lack of antigen presenting cells, and inhibition of NK cell activity via the local production of immunosuppressive factors and reduced levels of MHC class I expression (9). Thus, the notion of a ‘collapse of immune privilege’ has also been invoked as part of the mechanism by which AA can arise. Support for a genetic basis for AA comes from multiple lines of evidence, including the observed heritability in first degree relatives (10, 11), twin studies (12), and most recently, from the results of family-based linkage studies (13).

### Hypertrichosis

Body inherited hypertrichoses are rare human disorders characterized by excessive hair growth that do not depend on androgen stimulation. They are independent of age, gender, and ethnicity (P1). Hypertrichosis are often associated with additional anomalies including gingival hyperplasia, deafness, cardiomegaly, and bone abnormalities (P2). Without being bound by theory, inherited hypertrichoses represent examples of atavisms, or recurrence of an ancestral phenotype, where the genes that promote a full coat of hair in other mammals and were silenced throughout evolution have become “reactivated” in human hypertrichosis, invoking unusual genetic mechanisms to explain their occurrence (P3, P4).

Hypertrichosis syndromes fall under the larger umbrella of ectodermal dysplasias, which are characterized by abnormal development of the hair, skin, nails, teeth and/or eccrine glands. While these appendages vary greatly in their shape and function, they share several common developmental features, namely, formation through a series of interactions between the epithelia and adjacent mesenchyme during embryogenesis. Interestingly, many additional anomalies are associated with hypertrichosis. Members of the X-linked hypertrichosis family identified also exhibit dental anomalies and deafness. Moreover, generalized hypertrichosis terminalis is often associated with gingival hyperplasia; Cantu syndrome is additionally characterized by skeletal dysplasia and cardiomegaly; and Ambras syndrome patients commonly present with facial dysmorphologies and bone abnormalities. Despite the wide range phenotypes of these syndromes, the causative mechanism(s) underlying human hypertrichoses remain unknown.

### Treatment of Hair Growth Disorders

This invention provides for the discovery that an inhibitor of FGF13 can be used for the treatment of hair loss disorders. Non-limiting examples of hair loss disorders include: androgenetic alopecia, Alopecia areata, telogen effluvium, alopecia areata, alopecia totalis, and alopecia universalis. Androgenetic alopecia (also called anrogenic alopecia in women) is a common form of hair-loss in both men and women resulting in hair thinning and baldness of the scalp. For example, alopecia areata (AA), typically begins with patches of hair-loss on the scalp or other parts of the body. If AA is not treated or is not responsive to the treatments, then baldness in the affected area can result (e.g., alopecia totalis). Alopecia totalis (AT) as well as alopecia universalis (AU) are severe forms of alopecia areata (AA). AU is the most severe form of alopecia areata. See, e.g., Cho et al. (2012) J Korean Med Sci, 27: 799-802.

An aspect of the invention encompasses a method of treating a hair-loss disorder in a mammalian subject in need thereof, the method comprising administering to the subject an inhibitor of FGF13. In one embodiment, the hair-loss disorder comprises androgenetic alopecia, telogen effluvium, alopecia areata, tinea capitis, alopecia totalis, hypotrichosis, hereditary hypotrichosis simplex, or alopecia universalis. In one embodiment, the method further comprises the step (b) determining whether the inhibitor administered induced hair growth in the subject afflicted with a hair loss disorder as compared to the subject's hair growth prior to treatment with the inhibitor. In one embodiment, the inhibitor comprises an antibody that specifically binds to a protein comprising SEQ ID NO: 1, 3, 5, 7, 9, or 11. In another embodiment, the inhibitor is an antisense RNA that specifically inhibits expression of the gene that encodes the FGF13 protein; a siRNA that specifically targets the gene that encodes the FGF13 protein, or a small molecule. In one embodiment, the siRNA is directed to a human nucleic acid sequence comprising SEQ ID NO: 2, 4, 6, 8, 10, or 12. In another embodiment, the siRNA directed to a FGF13 gene comprises any one of the sequences listed in Table 1.

Another aspect of the invention encompasses a method for inducing hair growth in a subject, the method comprising administering to the subject an effective amount of an inhibitor of FGF13, thereby controlling hair growth in the subject. In one embodiment, the subject is afflicted with a hair-loss disorder. In another embodiment, the hair-loss disorder comprises androgenetic alopecia, telogen effluvium, alopecia areata, tinea capitis, alopecia totalis, hypotrichosis, hereditary hypotrichosis simplex, or alopecia universalis. In one embodiment, the method further comprises the step (b) determining whether the inhibitor administered induced hair growth in the subject afflicted with a hair loss disorder as compared to the subject's hair growth prior to treatment with the inhibitor. In one embodiment, the inhibitor comprises an antibody that specifically binds to a protein comprising SEQ ID NO: 1, 3, 5, 7, 9, or 11. In another embodiment, the inhibitor is an antisense RNA that specifically inhibits expression of the gene that encodes the FGF13 protein; a siRNA that specifically targets the gene that encodes the FGF13 protein, or a small molecule. In one embodiment, the siRNA is directed to a human nucleic acid sequence comprising SEQ ID NO: 2, 4, 6, 8, 10, or 12. In another embodiment, the siRNA directed to a FGF13 gene is any one of the sequences listed in Table 1.

### Treatment of Hair Growth Disorders

Another aspect of the invention encompasses a method of treating a hair-growth disorder in a mammalian subject in need thereof, the method comprising administering to the subject an activator of FGF13. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and autosomal recessive trichomegaly. In one embodiment, the activator is a polypeptide comprising SEQ ID NO: 1, 3, 5, 7, 9, or 11, or a fragment thereof; or a peptidomimetic comprising SEQ ID NO: 1, 3, 5, 7, 9, or 11.

Another aspect of the invention encompasses a method for reducing hair growth in a subject, the method comprising administering to the subject an effective amount of an activator of FGF13, thereby controlling hair growth in the subject. In one embodiment, the subject is afflicted with a hair-growth disorder. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and autosomal recessive trichomegaly. In another embodiment, the method further comprises the step (b) determining whether the activator administered reduced hair growth in the subject afflicted with a hair-growth disorder as compared to the subject's hair growth prior to treatment with the activator. In one embodiment, the activator is a polypeptide comprising SEQ ID NO: 1, 3, 5, 7, 9, or 11, or a fragment thereof; or a peptidomimetic comprising SEQ ID NO: 1, 3, 5, 7, 9, or 11.

Another aspect of the invention encompasses a method for reducing hair growth in a subject, the method comprising administering to the subject an effective amount of a FGF13 protein, thereby controlling hair growth in the subject. In one embodiment, the subject is afflicted with a hair-growth disorder. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and autosomal recessive trichomegaly. In another embodiment, the method further comprises the step (b) determining whether the FGF13 protein administered reduced hair growth in the subject afflicted with a hair-growth disorder as compared to the subject's hair growth prior to treatment with the protein.

### Treatment of Hair Disorders

Another aspect of the invention encompasses a method of treating a hair disorder in a mammalian subject in need thereof, the method comprising administering to the subject a compound that modulates the expression of FGF13. In one embodiment, the hair disorder is a hair-loss disorder. In one embodiment, the hair-loss disorder comprises androgenetic alopecia, telogen effluvium, alopecia areata, tinea capitis, alopecia totalis, hypotrichosis, hereditary hypotrichosis simplex, or alopecia universalis. In another embodiment, the hair disorder is a hair-growth disorder. In one embodiment, the hair-growth disorder comprises X-linked hypertrichosis, generalized hypertrichosis terminalis with or without gingival hyperplasia, autosomal recessive hypertrichosis, Cantu syndrome, Ambras type hypertrichosis and autosomal recessive trichomegaly. In one embodiment, the method further comprises the step (b) determining whether the compound administered induced hair growth in the subject afflicted with a hair loss disorder as compared to the subject's hair growth prior to treatment with the compound. In another embodiment, the method further comprises the step (b) determining whether the compound administered reduced hair growth in the subject afflicted with a hair-growth disorder as compared to the subject's hair growth prior to treatment with the compound.

In a further embodiment, the administering comprises a subcutaneous, intra-muscular, intra-peritoneal, or intravenous injection; an infusion; oral, nasal, or topical delivery; or a combination thereof. In some embodiments, the administering occurs daily, weekly, twice weekly, monthly, twice monthly, or yearly.

### DNA and Amino Acid Manipulation Methods and Purification Thereof

The practice of aspects of the present invention can employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual, 3^{rd} Ed., ed. by Sambrook (2001), Fritsch and Maniatis (Cold Spring Harbor Laboratory Press: 1989); DNA Cloning, Volumes I and II (D. N. Glover ed., 1985); Oljgonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture Of Animal Cells (R. I. Freshney, Alan R. Liss, Inc., 1987); Immobilized Cells and Enzymes (IRL Press, 1986); B. Perbal, A Practical Guide To Molecular Cloning (1984); the series, Methods In Enzymology (Academic Press, Inc., N.Y.), specifically, Methods In Enzymology, Vols. 154 and 155 (Wu et al. eds.); Gene Transfer Vectors For Mammalian Cells (J. H. Miller and M. P. Calos eds., 1987, Cold Spring Harbor Laboratory); Immunochemical Methods In Cell And Molecular Biology (Caner and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). All patents, patent applications and references cited herein are incorporated by reference in their entireties.

One skilled in the art can obtain a protein in several ways, which include, but are not limited to, isolating the protein via biochemical means or expressing a nucleotide sequence encoding the protein of interest by genetic engineering methods.

A protein is encoded by a nucleic acid (including, for example, genomic DNA, complementary DNA (cDNA), synthetic DNA, as well as any form of corresponding RNA). For example, it can be encoded by a recombinant nucleic acid of a gene. The proteins of the invention can be obtained from various sources and can be produced according to various techniques known in the art. For example, a nucleic acid that encodes a protein can be obtained by screening DNA libraries, or by amplification from a natural source. A protein can be a fragment or portion thereof. The nucleic acids encoding a protein can be produced via recombinant DNA technology and such recombinant nucleic acids can be prepared by conventional techniques, including chemical synthesis, genetic engineering, enzymatic techniques, or a combination thereof. For example, a FGF13 protein is the polypeptide encoded by the nucleic acid having the nucleotide sequence shown in SEQ ID NO: 2. An example of a FGF13 polypeptide has the amino acid sequence shown in SEQ ID NO: 1.

The FGF13 protein is encoded by the FGF13 gene (having Gene ID accession no. 2258) is a member of the fibroblast growth factor (FGF) family. FGF family members possess broad mitogenic and cell survival activities, and are involved in a variety of biological processes, including embryonic development, cell growth, morphogenesis, tissue repair, tumor growth, and invasion.

The polypeptide sequence of human FGF13 (transcript variant 1) is depicted in SEQ ID NO: 1. The nucleotide sequence of human FGF13 (transcript variant 1) is shown in SEQ ID NO: 2. Sequence information related to FGF13 (transcript variant 1) is accessible in public databases by GenBank Accession numbers NP_{—}004105.1 (protein) and NM 004114 (nucleic acid).

SEQ ID NO: 1 is the human wild type amino acid sequence corresponding to FGF13 isoform 1 (residues 1-245):

| | |
|---|---|
| 1 | MAAAIASSLI RQKRQARERE KSNACKCVSS PSKGKTSCDK NKLNVFSRVK LFGSKKRRRR |
| 61 | RPEPQLKGIV TKLYSRQGYH LQLQADGTID GTKDEDSTYT LFNLIPVGLR VVAIQGVQTK |
| 121 | LYLAMNSEGY LYTSELFTPE CKFKESVFEN YYVTYSSMIY RQQQSGRGWY LGLNKEGEIM |
| 181 | KGNHVKKNKP AAHFLPKPLK VAMYKEPSLH DLTEFSRSGS GTPTKSRSVS GVLNGGKSMS |
| 241 | HNEST |

SEQ ID NO: 2 is the human wild type nucleotide sequence corresponding to FGF13 (transcript variant 1) (nucleotides 1-2705), wherein the underscored bolded “ATG” denotes the beginning of the open reading frame:

| | |
|---|---|
| 1 | gtgccgcgcc cagagcagca gcaacagcga agatgcgagg ccattacctg tttgatccct |
| 61 | gtcggaaacc tggcacgggc caacttttcc cgattatcac gccaagaagt tgcaaggact |
| 121 | agtcgaagac tcggaggggc cagggcgagg gcgcgctccc ccgcgcgctg cctcatccct |
| 181 | cctccgtccg gccgcccgag ctcccggcct ctctcccgcc cgcgctcact ccctccgccc |
| 241 | gcctccctcc tctggccccc atcagaaggg caacagggcg agggggtccg gcgaaattcg |
| 301 | gaccggagca gctggacatg cacggtgtcc gccgggcgca ggggccgacc acacgcagtc |
| 361 | gcgcagttca gcatccgcgt gccagtctcg cccgcgatcc cgggcccggg gctgtggcgt |
| 421 | cgactccgac ccaggcagcc agcagcccgc gcgggagccg gaccgccgcc ggaggagctc |
| 481 | ggacggcatg ctgagccccc tccttggctg aagcccgagt gcggagaagc ccgggcaaac |
| 541 | gcaggctaag gagaccaaag cggcgaagtc gcgagacagc ggacaagcag cggaggagaa |
| 601 | ggaggaggag gcgaacccag agaggggcag caaaagaagc ggtggtggtg ggcgtcgtgg |
| 661 | ccatggcggc ggctatcgcc agctcgctca tccgtcagaa gaggcaagcc cgcgagcgcg |
| 721 | agaaatccaa cgcctgcaag tgtgtcagca gccccagcaa aggcaagacc agctgcgaca |
| 781 | aaaacaagtt aaatgtcttt tcccgggtca aactcttcgg ctccaagaag aggcgcagaa |
| 841 | gaagaccaga gcctcagctt aagggtatag ttaccaagct atacagccga caaggctacc |
| 901 | acttgcagct gcaggcggat ggaaccattg atggcaccaa agatgaggac agcacttaca |
| 961 | ctctgtttaa cctcatccct gtgggtctgc gagtggtggc tatccaagga gttcaaacca |
| 1021 | agctgtactt ggcaatgaac agtgagggat acttgtacac ctcggaactt ttcacacctg |
| 1081 | agtgcaaatt caaagaatca gtgtttgaaa attattatgt gacatattca tcaatgatat |
| 1141 | accgtcagca gcagtcaggc cgagggtggt atctgggtct gaacaaagaa ggagagatca |
| 1201 | tgaaaggcaa ccatgtgaag aagaacaagc ctgcagctca ttttctgcct aaaccactga |
| 1261 | aagtggccat gtacaaggag ccatcactgc acgatctcac ggagttctcc cgatctggaa |
| 1321 | gcgggacccc aaccaagagc agaagtgtct ctggcgtgct gaacggaggc aaatccatga |
| 1381 | gccacaatga atcaacgtag ccagtgaggg caaaagaagg gctctgtaac agaaccttac |
| 1441 | ctccaggtgc tgttgaattc ttctagcagt ccttcaccca aaagttcaaa tttgtcagtg |
| 1501 | acatttacca aacaaacagg cagagttcac tattctatct gccattagac cttcttatca |
| 1561 | tccatactaa agccccatta tttagattga gcttgtgcat aagaatgcca agcattttag |
| 1621 | tgaactaaat ctgagagaag gactgccaaa ttttctcatg atctcaccta tactttgggg |
| 1681 | atgataatcc aaaagtattt cacagcacta atgctgatca aaatttgctc tcccaccaag |
| 1741 | aaaatgtaaa agaccacaat tgttcttcaa aaacaaacaa aacaaaacaa aacaaaatta |
| 1801 | actgcttaaa tgttttgtcg gggcaaacaa aattatgtga attgtgttgt tttcttggct |
| 1861 | tgatgttttc tatctacgct tgattcacat gtactctttt ctttggcata gtgcaacttt |
| 1921 | atgatttctg aaattcaatg gttctattga ctttttgcgt cacttaatcc aaatcaacca |
| 1981 | aattcagggt tgaatctgaa ttggcttctc aggctcaagg taacagtgtt cttgtggttt |
| 2041 | gaccaattgt ttttctttct ttttctttct ttttagattt gtggtattct ggtcaagtta |
| 2101 | ttgtgctgta ctttgtgcgt agaaattgag ttgtattgtc aaccccagtc agtaaagaga |
| 2161 | acttcaaaaa attatcctca agtgtagatt tctcttaatt ccatttgtgt atcatgttaa |
| 2221 | actattgttg tggcttcttg tgtaaagaca ggaactgtgg aactgtgatg ttgtcttttg |
| 2281 | tgttgttaaa ataagaaatg tcttatctgt atatgtatga gtcttcctgt cattgtattt |
| 2341 | ggcacatgaa tattgtgtac aaggaattgt taagactggt tttccctcaa caacatatat |
| 2401 | tatacttgct actggaaaag tgtttaagac ttagctaggt ttccatttag atcttcatat |
| 2461 | ctgttgcatg gaagaaagtt gggttcttgg catagagttg catgatatgt aagattttgt |
| 2521 | gcattcataa ttgttaaaaa tctgtgttcc aaaagtggac atagcatgta caggcagttt |
| 2581 | tctgtcctgt gcacaaaaag tttaaaaaag ttgtttaata tttgttgttg tatacccaaa |
| 2641 | tacgcaccga ataaactctt tatattcatt caaagaaaaa aaaaaaaaaa aaaaaaaaaa |
| 2701 | aaaaa |

The polypeptide sequence of human FGF13 (transcript variant 2) is depicted in SEQ ID NO: 3. The nucleotide sequence of human FGF13 (transcript variant 2) is shown in SEQ ID NO: 4. Sequence information related to FGF13 (transcript variant 2) is accessible in public databases by GenBank Accession numbers NP_{—}001132972 (protein) and NM_{—}001139500 (nucleic acid).

SEQ ID NO: 3 is the human wild type amino acid sequence corresponding to FGF13 isoform 2 (residues 1-255):

| | |
|---|---|
| 1 | MSGKVTKPKE EKDASKVLDD APPGTQEYIM LRQDSIQSAE LKKKESPFRA KCHEIFCCPL |
| 61 | KQVHHKENTE PEEPQLKGIV TKLYSRQGYH LQLQADGTID GTKDEDSTYT LFNLIPVGLR |
| 121 | VVAIQGVQTK LYLAMNSEGY LYTSELFTPE CKFKESVFEN YYVTYSSMIY RQQQSGRGWY |
| 181 | LGLNKEGEIM KGNHVKKNKP AAHFLPKPLK VAMYKEPSLH DLTEFSRSGS GTPTKSRSVS |
| 241 | GVLNGGKSMS HNEST |

SEQ ID NO: 4 is the human wild type nucleotide sequence corresponding to FGF13 (transcript variant 2) (nucleotides 1-2340), wherein the underscored bolded “ATG” denotes the beginning of the open reading frame:

| | |
|---|---|
| 1 | gtggctctct aggaccggag agttctttgg aaggagagcg cgagcgaggg agcgggcgag |
| 61 | ctccgagggg gtgtgggtgt agggagagag agaaagagag caggcagcgg cggcggcggc |
| 121 | agcggtgggg aaaagcggat tccgccccga accacaccga ggggagctcg tggtcgagac |
| 181 | ttgccgccct aagcactctc ccaagtccga cccgctcggc gaggacttcc gtcttctgag |
| 241 | cgaaccttgt caagcaagct gggatctatg agtggaaagg tgaccaagcc caaagaggag |
| 301 | aaagatgctt ctaaggttct ggatgacgcc ccccctggca cacaggaata cattatgtta |
| 361 | cgacaagatt ccatccaatc tgcggaatta aagaaaaaag agtccccctt tcgtgctaag |
| 421 | tgtcacgaaa tcttctgctg cccgctgaag caagtacacc acaaagagaa cacagagccg |
| 481 | gaagagcctc agcttaaggg tatagttacc aagctataca gccgacaagg ctaccacttg |
| 541 | cagctgcagg cggatggaac cattgatggc accaaagatg aggacagcac ttacactctg |
| 601 | tttaacctca tccctgtggg tctgcgagtg gtggctatcc aaggagttca aaccaagctg |
| 661 | tacttggcaa tgaacagtga gggatacttg tacacctcgg aacttttcac acctgagtgc |
| 721 | aaattcaaag aatcagtgtt tgaaaattat tatgtgacat attcatcaat gatataccgt |
| 781 | cagcagcagt caggccgagg gtggtatctg ggtctgaaca aagaaggaga gatcatgaaa |
| 841 | ggcaaccatg tgaagaagaa caagcctgca gctcattttc tgcctaaacc actgaaagtg |
| 901 | gccatgtaca aggagccatc actgcacgat ctcacggagt tctcccgatc tggaagcggg |
| 961 | accccaacca agagcagaag tgtctctggc gtgctgaacg gaggcaaatc catgagccac |
| 1021 | aatgaatcaa cgtagccagt gagggcaaaa gaagggctct gtaacagaac cttacctcca |
| 1081 | ggtgctgttg aattcttcta gcagtccttc acccaaaagt tcaaatttgt cagtgacatt |
| 1141 | taccaaacaa acaggcagag ttcactattc tatctgccat tagaccttct tatcatccat |
| 1201 | actaaagccc cattatttag attgagcttg tgcataagaa tgccaagcat tttagtgaac |
| 1261 | taaatctgag agaaggactg ccaaattttc tcatgatctc acctatactt tggggatgat |
| 1321 | aatccaaaag tatttcacag cactaatgct gatcaaaatt tgctctccca ccaagaaaat |
| 1381 | gtaaaagacc acaattgttc ttcaaaaaca aacaaaacaa aacaaaacaa aattaactgc |
| 1441 | ttaaatgttt tgtcggggca aacaaaatta tgtgaattgt gttgttttct tggcttgatg |
| 1501 | ttttctatct acgcttgatt cacatgtact cttttctttg gcatagtgca actttatgat |
| 1561 | ttctgaaatt caatggttct attgactttt tgcgtcactt aatccaaatc aaccaaattc |
| 1621 | agggttgaat ctgaattggc ttctcaggct caaggtaaca gtgttcttgt ggtttgacca |
| 1681 | attgtttttc tttctttttt ttttttttta gatttgtggt attctggtca agttattgtg |
| 1741 | ctgtactttg tgcgtagaaa ttgagttgta ttgtcaaccc cagtcagtaa agagaacttc |
| 1801 | aaaaaattat cctcaagtgt agatttctct taattccatt tgtgtatcat gttaaactat |
| 1861 | tgttgtggct tcttgtgtaa agacaggaac tgtggaactg tgatgttgtc ttttgtgttg |
| 1921 | ttaaaataag aaatgtctta tctgtatatg tatgagtctt cctgtcattg tatttggcac |
| 1981 | atgaatattg tgtacaagga attgttaaga ctggttttcc ctcaacaaca tatattatac |
| 2041 | ttgctactgg aaaagtgttt aagacttagc taggtttcca tttagatctt catatctgtt |
| 2101 | gcatggaaga aagttgggtt cttggcatag agttgcatga tatgtaagat tttgtgcatt |
| 2161 | cataattgtt aaaaatctgt gttccaaaag tggacatagc atgtacaggc agttttctgt |
| 2221 | cctgtgcaca aaaagtttaa aaaagttgtt taatatttgt tgttgtatac ccaaatacgc |
| 2281 | accgaataaa ctctttatat tcattcaaag aaaaaaaaaa aaaaaaaaaa aaaaaaaaaa |

The polypeptide sequence of human FGF13 (transcript variant 3) is depicted in SEQ ID NO: 5. The nucleotide sequence of human FGF13 (transcript variant 3) is shown in SEQ ID NO: 6. Sequence information related to FGF13 (transcript variant 3) is accessible in public databases by GenBank Accession numbers NP_{—}001132973 (protein) and N114_{—}001139501 (nucleic acid).

SEQ ID NO: 5 is the human wild type amino acid sequence corresponding to FGF13 isoform 3 (residues 1-226):

| | |
|---|---|
| 1 | MLRQDSIQSA ELKKKESPFR AKCHEIFCCP LKQVHHKENT EPEEPQLKGI VTKLYSRQGY |
| 61 | HLQLQADGTI DGTKDEDSTY TLFNLIPVGL RVVAIQGVQT KLYLAMNSEG YLYTSELFTP |
| 121 | ECKFKESVFE NYYVTYSSMI YRQQQSGRGW YLGLNKEGEI MKGNHVKKNK PAAHFLPKPL |
| 181 | KVAMYKEPSL HDLTEFSRSG SGTPTKSRSV SGVLNGGKSM SHNEST |

SEQ ID NO: 6 is the human wild type nucleotide sequence corresponding to FGF13 (transcript variant 3) (nucleotides 1-2450), wherein the underscored bolded “ATG” denotes the beginning of the open reading frame:

| | |
|---|---|
| 1 | gtggctctct aggaccggag agttctttgg aaggagagcg cgagcgaggg agcgggcgag |
| 61 | ctccgagggg gtgtgggtgt agggagagag agaaagagag caggcagcgg cggcggcggc |
| 121 | agcggtgggg aaaagcggat tccgccccga accacaccga ggggagctcg tggtcgagac |
| 181 | ttgccgccct aagcactctc ccaagtccga cccgctcggc gaggacttcc gtcttctgag |
| 241 | cgaaccttgt caagcaagct gggatctatg agtggaaagg tgaccaagcc caaagaggag |
| 301 | aaagatgctt ctaagggagt ttctctgcac aagctctctg tttgcctgct gtcgtccaca |
| 361 | taagatgtga cttgctcctg cttgccttcc tccatgattg tgaggcctcc ccagccacgt |
| 421 | ggaactttct ggatgacgcc ccccctggca cacaggaata cattatgtta cgacaagatt |
| 481 | ccatccaatc tgcggaatta aagaaaaaag agtccccctt tcgtgctaag tgtcacgaaa |
| 541 | tcttctgctg cccgctgaag caagtacacc acaaagagaa cacagagccg gaagagcctc |
| 601 | agcttaaggg tatagttacc aagctataca gccgacaagg ctaccacttg cagctgcagg |
| 661 | cggatggaac cattgatggc accaaagatg aggacagcac ttacactctg tttaacctca |
| 721 | tccctgtggg tctgcgagtg gtggctatcc aaggagttca aaccaagctg tacttggcaa |
| 781 | tgaacagtga gggatacttg tacacctcgg aacttttcac acctgagtgc aaattcaaag |
| 841 | aatcagtgtt tgaaaattat tatgtgacat attcatcaat gatataccgt cagcagcagt |
| 901 | caggccgagg gtggtatctg ggtctgaaca aagaaggaga gatcatgaaa ggcaaccatg |
| 961 | tgaagaagaa caagcctgca gctcattttc tgcctaaacc actgaaagtg gccatgtaca |
| 1021 | aggagccatc actgcacgat ctaagggagt tctcccgatc tggaagcggg accccaacca |
| 1081 | agagcagaag tgtctctggc gtgctgaacg gaggcaaatc catgagccac aatgaatcaa |
| 1141 | cgtagccagt gagggcaaaa gaagggctct gtaacagaac cttacctcca ggtgctgttg |
| 1201 | aattcttcta gcagtccttc acccaaaagt tcaaatttgt cagtgacatt taccaaacaa |
| 1261 | acaggcagag ttcactattc tatctgccat tagaccttct tatcatccat actaaagccc |
| 1321 | cattatttag attgagcttg tgcataagaa tgccaagcat tttagtgaac taaatctgag |
| 1381 | agaaggactg ccaaattttc tcatgatctc acctatactt tggggatgat aatccaaaag |
| 1441 | tatttcacag cactaatgct gatcaaaatt tgctctccca ccaagaaaat gtaaaagacc |
| 1501 | acaattgttc ttcaaaaaca aacaaaacaa aacaaaacaa aattaactgc ttaaatgttt |
| 1561 | tgtcggggca aacaaaatta tgtgaattgt gttgttttct tggcttgatg ttttctatct |
| 1621 | acgcttgatt cacatgtact cttttctttg gcatagtgca actttatgat ttctgaaatt |
| 1681 | caatggttct attgactttt tgcgtcactt aatccaaatc aaccaaattc agggttgaat |
| 1741 | ctgaattggc ttctcaggct caaggtaaca gtgttcttgt ggtttgacca attgtttttc |
| 1801 | tttctttttt ttttttttta gatttgtggt attctggtca agttattgtg ctgtactttg |
| 1861 | tgcgtagaaa ttgagttgta ttgtcaaccc cagtcagtaa agagaacttc aaaaaattat |
| 1921 | cctcaagtgt agatttctct taattccatt tgtgtatcat gttaaactat tgttgtggct |
| 1981 | tcttgtgtaa agacaggaac tgtggaactg tgatgttgtc ttttgtgttg ttaaaataag |
| 2041 | aaatgtctta tctgtatatg tatgagtctt cctgtcattg tatttggcac atgaatattg |
| 2101 | tgtacaagga attgttaaga ctggttttcc ctcaacaaca tatattatac ttgctactgg |
| 2161 | aaaagtgttt aagacttagc taggtttcca tttagatctt catatctgtt gcatggaaga |
| 2221 | aagttgggtt cttggcatag agttgcatga tatgtaagat tttgtgcatt cataattgtt |
| 2281 | aaaaatctgt gttccaaaag tggacatagc atgtacaggc agttttctgt cctgtgcaca |
| 2341 | aaaagtttaa aaaagttgtt taatatttgt tgttgtatac ccaaatacgc accgaataaa |
| 2401 | ctctttatat tcattcaaag aaaaaaaaaa aaaaaaaaaa aaaaaaaaaa |

The polypeptide sequence of human FGF13 (transcript variant 4) is depicted in SEQ ID NO: 7. The nucleotide sequence of human FGF13 (transcript variant 4) is shown in SEQ ID NO: 8. Sequence information related to FGF13 (transcript variant 4) is accessible in public databases by GenBank Accession numbers NP_{—}001132970 (protein) and NM_{—}001139498 (nucleic acid).

SEQ ID NO: 7 is the human wild type amino acid sequence corresponding to FGF13 isoform 4 (residues 1-199):

| | |
|---|---|
| 1 | MSGKVTKPKE EKDASKEPQL KGIVTKLYSR QGYHLQLQAD GTIDGTKDED STYTLFNLIP |
| 61 | VGLRVVAIQG VQTKLYLAMN SEGYLYTSEL FTPECKFKES VFENYYVTYS SMIYRQQQSG |
| 121 | RGWYLGLNKE GEIMKGNHVK KNKPAAHFLP KPLKVAMYKE PSLEDLTEFS RSGSGTPTKS |
| 181 | RSVSGVLNGG KSMSHNEST |

SEQ ID NO: 8 is the human wild type nucleotide sequence corresponding to FGF13 (transcript variant 4) (nucleotides 1-2172), wherein the underscored bolded “ATG” denotes the beginning of the open reading frame:

| | |
|---|---|
| 1 | gtggctctct aggaccggag agttctttgg aaggagagcg cgagcgaggg agcgggcgag |
| 61 | ctccgagggg gtgtgggtgt agggagagag agaaagagag caggcagcgg cggcggcggc |
| 121 | agcggtgggg aaaagcggat tccgccccga accacaccga ggggagctcg tggtcgagac |
| 181 | ttgccgccct aagcactctc ccaagtccga cccgctcggc gaggacttcc gtcttctgag |
| 241 | cgaaccttgt caagcaagct gggatctatg agtggaaagg tgaccaagcc caaagaggag |
| 301 | aaagatgctt ctaaggagcc tcagcttaag ggtatagtta ccaagctata cagccgacaa |
| 361 | ggctaccact tgcagctgca ggcggatgga accattgatg gcaccaaaga tgaggacagc |
| 421 | acttacactc tgtttaacct catccctgtg ggtctgcgag tggtggctat ccaaggagtt |
| 481 | caaaccaagc tgtacttggc aatgaacagt gagggatact tgtacacctc ggaacttttc |
| 541 | acacctgagt gcaaattcaa agaatcagtg tttgaaaatt attatgtgac atattcatca |
| 601 | atgatatacc gtcagcagca gtcaggccga gggtggtatc tgggtctgaa caaagaagga |
| 661 | gagatcatga aaggcaacca tgtgaagaag aacaagcctg cagctcattt tctgcctaaa |
| 721 | ccactgaaag tggccatgta caaggagcca tcactgcacg atctcacgga gttctcccga |
| 781 | tctggaagcg ggaccccaac caagagcaga agtgtctctg gcgtgctgaa cggaggcaaa |
| 841 | tccatgagcc acaatgaatc aacgtagcca gtgagggcaa aagaagggct ctgtaacaga |
| 901 | accttacctc caggtgctgt tgaattcttc tagcagtcct tcacccaaaa gttcaaattt |
| 961 | gtcagtgaca tttaccaaac aaacaggcag agttcactat tctatctgcc attagacctt |
| 1021 | cttatcatcc atactaaagc cccattattt agattgagct tgtgcataag aatgccaagc |
| 1081 | attttagtga actaaatctg agagaaggac tgccaaattt tctcatgatc tcacctatac |
| 1141 | tttggggatg ataatccaaa agtatttcac agcactaatg ctgatcaaaa tttgctctcc |
| 1201 | caccaagaaa atgtaaaaga ccacaattgt tcttcaaaaa caaacaaaac aaaacaaaac |
| 1261 | aaaattaact gcttaaatgt tttgtcgggg caaacaaaat tatgtgaatt gtgttgtttt |
| 1321 | cttggcttga tgttttctat ctacgcttga ttcacatgta ctcttttctt tggcatagtg |
| 1381 | caactttatg atttctgaaa ttcaatggtt ctattgactt tttgcgtcac ttaatccaaa |
| 1441 | tcaaccaaat tcagggttga atctgaattg gcttctcagg ctcaaggtaa cagtgttctt |
| 1501 | gtggtttgac caattgtttt tctttctttt tttttttttt tagatttgtg gtattctggt |
| 1561 | caagttattg tgctgtactt tgtgcgtaga aattgagttg tattgtcaac cccagtcagt |
| 1621 | aaagagaact tcaaaaaatt atcctcaagt gtagatttct cttaattcca tttgtgtatc |
| 1681 | atgttaaact attgttgtgg cttcttgtgt aaagacagga actgtggaac tgtgatgttg |
| 1741 | tcttttgtgt tgttaaaata agaaatgtct tatctgtata tgtatgagtc ttcctgtcat |
| 1801 | tgtatttggc acatgaatat tgtgtacaag gaattgttaa gactggtttt ccctcaacaa |
| 1861 | catatattat acttgctact ggaaaagtgt ttaagactta gctaggtttc catttagatc |
| 1921 | ttcatatctg ttgcatggaa gaaagttggg ttcttggcat agagttgcat gatatgtaag |
| 1981 | attttgtgca ttcataattg ttaaaaatct gtgttccaaa agtggacata gcatgtacag |
| 2041 | gcagttttct gtcctgtgca caaaaagttt aaaaaagttg tttaatattt gttgttgtat |
| 2101 | acccaaatac gcaccgaata aactctttat attcattcaa agaaaaaaaa aaaaaaaaaa |
| 2161 | aaaaaaaaaa aa |

The polypeptide sequence of human FGF13 (transcript variant 5) is depicted in SEQ ID NO: 9. The nucleotide sequence of human FGF13 (transcript variant 5) is shown in SEQ ID NO: 10. Sequence information related to FGF13 (transcript variant 5) is accessible in public databases by GenBank Accession numbers NP_{—}001132974 (protein) and NM_{—}001139502 (nucleic acid).

SEQ ID NO: 9 is the human wild type amino acid sequence corresponding to FGF13 isoform 5 (residues 1-226):

| | |
|---|---|
| 1 | MLRQDSIQSA ELKKKESPFR AKCHEIFCCP LKQVHHKENT EPEEPQLKGI VTKLYSRQGY |
| 61 | HLQLQADGTI DGTKDEDSTY TLFNLIPVGL RVVAIQGVQT KLYLAMUSEG YLYTSELFTP |
| 121 | ECKFKESVFE NYYVTYSSMI YRQQQSGRGW YLGLNKEGEI MKGNHVKKNK PAAHFLPKPL |
| 181 | KVAMYKEPSL HDLTEFSRSG SGTPTKSRSV SGVLNGGKSM SHNEST |

SEQ ID NO: 10 is the human wild type nucleotide sequence corresponding to FGF13 (transcript variant 5) (nucleotides 1-2093), wherein the underscored bolded “ATG” denotes the beginning of the open reading frame:

| | |
|---|---|
| 1 | catgtaacat gtgatttgct cctccttgcc ttccaccgtg atgtgaggcc tccccaacca |
| 61 | agtggaactt tctggatgac gccccccctg gcacacagga atacattatg ttacgacaag |
| 121 | attccatcca atctgcggaa ttaaagaaaa aagagtcccc ctttcgtgct aagtgtcacg |
| 181 | aaatcttctg ctgcccgctg aagcaagtac accacaaaga gaacacagag ccggaagagc |
| 241 | ctcagcttaa gggtatagtt accaagctat acagccgaca aggctaccac ttgcagctgc |
| 301 | aggcggatgg aaccattgat ggcaccaaag atgaggacag cacttacact ctgtttaacc |
| 361 | tcatccctgt gggtctgcga gtggtggcta tccaaggagt tcaaaccaag ctgtacttgg |
| 421 | caatgaacag tgagggatac ttgtacacct cggaactttt cacacctgag tgcaaattca |
| 481 | aagaatcagt gtttgaaaat tattatgtga catattcatc aatgatatac cgtcagcagc |
| 541 | agtcaggccg agggtggtat ctgggtctga acaaagaagg agagatcatg aaaggcaacc |
| 601 | atgtgaagaa gaacaagcct gcagctcatt ttctgcctaa accactgaaa gtggccatgt |
| 661 | acaaggagcc atcactgcac gatctcacgg agttctcccg atctggaagc gggaccccaa |
| 721 | ccaagagcag aagtgtctct ggcgtgctga acggaggcaa atccatgagc cacaatgaat |
| 781 | caacgtagcc agtgagggca aaagaagggc tctgtaacag aaccttacct ccaggtgctg |
| 841 | ttgaattctt ctagcagtcc ttcacccaaa agttcaaatt tgtcagtgac atttaccaaa |
| 901 | caaacaggca gagttcacta ttctatctgc cattagacct tcttatcatc catactaaag |
| 961 | ccccattatt tagattgagc ttgtgcataa gaatgccaag cattttagtg aactaaatct |
| 1021 | gagagaagga ctgccaaatt ttctcatgat ctcacctata ctttggggat gataatccaa |
| 1081 | aagtatttca cagcactaat gctgatcaaa atttgctctc ccaccaagaa aatgtaaaag |
| 1141 | accacaattg ttcttcaaaa acaaacaaaa caaaacaaaa caaaattaac tgcttaaatg |
| 1201 | ttttgtcggg gcaaacaaaa ttatgtgaat tgtgttgttt tcttggcttg atgttttcta |
| 1261 | tctacgcttg attcacatgt actcttttct ttggcatagt gcaactttat gatttctgaa |
| 1321 | attcaatggt tctattgact ttttgcgtca cttaatccaa atcaaccaaa ttcagggttg |
| 1381 | aatctgaatt ggcttctcag gctcaaggta acagtgttct tgtggtttga ccaattgttt |
| 1441 | tttttttttt tttttttttt ttagatttgt ggtattctgg tcaagttatt gtgctgtact |
| 1501 | ttgtgcgtag aaattgagtt gtattgtcaa ccccagtcag taaagagaac ttcaaaaaat |
| 1561 | tatcctcaag tgtagatttc tcttaattcc atttgtgtat catgttaaac tattgttgtg |
| 1621 | gcttcttgtg taaagacagg aactgtggaa ctgtgatgtt gtcttttgtg ttgttaaaat |
| 1681 | aagaaatgtc ttatctgtat atgtatgagt cttcctgtca ttgtatttgg cacatgaata |
| 1741 | ttgtgtacaa ggaattgtta agactggttt tccctcaaca acatatatta tacttgctac |
| 1801 | tggaaaagtg tttaagactt agctaggttt ccatttagat cttcatatct gttgcatgga |
| 1861 | agaaagttgg gttcttggca tagagttgca tgatatgtaa gattttgtgc attcataatt |
| 1921 | gttaaaaatc tgtgttccaa aagtggacat agcatgtaca ggcagttttc tgtcctgtgc |
| 1981 | acaaaaagtt taaaaaagtt gtttaatatt tgttgttgta tacccaaata cgcaccgaat |
| 2041 | aaactcttta tattcattca aagaaaaaaa aaaaaaaaaa aaaaaaaaaa aaa |

The polypeptide sequence of human FGF13 (transcript variant 6) is depicted in SEQ ID NO: 11. The nucleotide sequence of human FGF13 (transcript variant 6) is shown in SEQ ID NO: 12. Sequence information related to FGF13 (transcript variant 6) is accessible in public databases by GenBank Accession numbers NP_{—}378668 (protein) and NM_{—}033642 (nucleic acid).

SEQ ID NO: 11 is the human wild type amino acid sequence corresponding to FGF13 isoform 6 (residues 1-192):

| | |
|---|---|
| 1 | MALLRKSYSE PQLKGIVTKL YSRQGYHLQL QADGTIDGTK DEDSTYTLFN LIPVGLRVVA |
| 61 | IQGVQTKLYL AMNSEGYLYT SELFTPECKF KESVFENYYV TYSSMIYRQQ QSGRGWYLGL |
| 121 | NKEGEIMKGN HVKKNKPAAH FLPKPLKVAM YKEPSLHDLT EFSRSGSGTP TKSRSVSGVL |
| 181 | NGGKSMSHNE ST |

SEQ ID NO: 12 is the human wild type nucleotide sequence corresponding to FGF13 (transcript variant 6) (nucleotides 1-1968), wherein the underscored bolded “ATG” denotes the beginning of the open reading frame:

| | |
|---|---|
| 1 | aaactttctc tgatctcctc tctctctgtg tctgctccaa atgtagacag caattgtctg |
| 61 | ggtaggacca gcttataaag aagcatggct ttgttaagga agtcgtattc agagcctcag |
| 121 | cttaagggta tagttaccaa gctatacagc cgacaaggct accacttgca gctgcaggcg |
| 181 | gatggaacca ttgatggcac caaagatgag gacagcactt acactctgtt taacctcatc |
| 241 | cctgtgggtc tgcgagtggt ggctatccaa ggagttcaaa ccaagctgta cttggcaatg |
| 301 | aacagtgagg gatacttgta cacctcggaa cttttcacac ctgagtgcaa attcaaagaa |
| 361 | tcagtgtttg aaaattatta tgtgacatat tcatcaatga tataccgtca gcagcagtca |
| 421 | ggccgagggt ggtatctggg tctgaacaaa gaaggagaga tcatgaaagg caaccatgtg |
| 481 | aagaagaaca agcctgcagc tcattttctg cctaaaccac tgaaagtggc catgtacaag |
| 541 | gagccatcac tgcacgatct cacggagttc tcccgatctg gaagcgggac cccaaccaag |
| 601 | agcagaagtg tctctggcgt gctgaacgga ggcaaatcca tgagccacaa tgaatcaacg |
| 661 | tagccagtga gggcaaaaga agggctctgt aacagaacct tacctccagg tgctgttgaa |
| 721 | ttcttctagc agtccttcac ccaaaagttc aaatttgtca gtgacattta ccaaacaaac |
| 781 | aggcagagtt cactattcta tctgccatta gaccttctta tcatccatac taaagcccca |
| 841 | ttatttagat tgagcttgtg cataagaatg ccaagcattt tagtgaacta aatctgagag |
| 901 | aaggactgcc aaattttctc atgatctcac ctatactttg gggatgataa tccaaaagta |
| 961 | tttcacagca ctaatgctga tcaaaatttg ctctcccacc aagaaaatgt aaaagaccac |
| 1021 | aattgttctt caaaaacaaa caaaacaaaa caaaacaaaa ttaactgctt aaatgttttg |
| 1081 | tcggggcaaa caaaattatg tgaattgtgt tgttttcttg gcttgatgtt ttctatctac |
| 1141 | gcttgattca catgtactct tttctttggc atagtgcaac tttatgattt ctgaaattca |
| 1201 | atggttctat tgactttttg cgtcacttaa tccaaatcaa ccaaattcag ggttgaatct |
| 1261 | gaattggctt ctcaggctca aggtaacagt gttcttgtgg tttgaccaat tgtttttctt |
| 1321 | tctttttttt tttttttaga tttgtggtat tctggtcaag ttattgtgct gtactttgtg |
| 1381 | cgtagaaatt gagttgtatt gtcaacccca gtcagtaaag agaacttcaa aaaattatcc |
| 1441 | tcaagtgtag atttctctta attccatttg tgtatcatgt taaactattg ttgtggcttc |
| 1501 | ttgtgtaaag acaggaactg tggaactgtg atgttgtctt ttgtgttgtt aaaataagaa |
| 1561 | atgtcttatc tgtatatgta tgagtcttcc tgtcattgta tttggcacat gaatattgtg |
| 1621 | tacaaggaat tgttaagact ggttttccct caacaacata tattatactt gctactggaa |
| 1681 | aagtgtttaa gacttagcta ggtttccatt tagatcttca tatctgttgc atggaagaaa |
| 1741 | gttgggttct tggcatagag ttgcatgata tgtaagattt tgtgcattca taattgttaa |
| 1801 | aaatctgtgt tccaaaagtg gacatagcat gtacaggcag ttttctgtcc tgtgcacaaa |
| 1861 | aagtttaaaa aagttgttta atatttgttg ttgtataccc aaatacgcac cgaataaact |
| 1921 | ctttatattc attcaaagaa aaaaaaaaaa aaaaaaaaaa aaaaaaaa |

Protein Variants:

Protein variants can include amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture.

Nucleic acid sequences comprising a gene, such as a FGF13 gene, that encodes a polypeptide can be synthesized, in whole or in part, using chemical methods known in the art. Alternatively, a polypeptide, such as FGF13, can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of FGF13 polypeptides can be separately synthesized and combined using chemical methods to produce a full-length molecule.

The nucleic acid can be any type of nucleic acid, including genomic DNA, complementary DNA (cDNA), synthetic or semi-synthetic DNA, as well as any form of corresponding RNA. For example, a FGF13 molecule can comprise a recombinant nucleic acid encoding human FGF13 protein. In one embodiment, a FGF13 molecule can comprise a non-naturally occurring nucleic acid created artificially (such as by assembling, cutting, ligating or amplifying sequences). A FGF13 molecule can be double-stranded. A FGF13 molecule can be single-stranded. The FGF13 molecules of the invention can be obtained from various sources and can be produced according to various techniques known in the art. For example, a nucleic acid that is a FGF13 molecule can be obtained by screening DNA libraries, or by amplification from a natural source. The FGF13 molecules can be produced via recombinant DNA technology and such recombinant nucleic acids can be prepared by conventional techniques, including chemical synthesis, genetic engineering, enzymatic techniques, or a combination thereof. Non-limiting examples of a FGF13 molecule that is a nucleic acid, is the nucleic acid comprising SEQ ID NO: 2. Another example of a FGF13 molecule is a fragment of a nucleic acid comprising the sequence shown in SEQ ID NO: 2, wherein the fragment exhibits FGF13 activity. A FGF13 molecule of this invention also encompasses variants of the human nucleic acid encoding the FGF13 protein, or variants of the human FGF13 proteins that exhibit FGF13 activity. A FGF13 molecule can also include a fragment of the human FGF13 nucleic acid which encodes a polypeptide that exhibits FGF13 activity. A FGF13 molecule can encompass a fragment of the human FGF13 protein that exhibits FGF13 activity.

A FGF13 molecule can also encompass FGF13 ortholog genes, which are genes conserved among different biological species such as humans, dogs, cats, mice, and rats, that encode proteins (for example, homologs (including splice variants), mutants, and derivatives) having biologically equivalent functions as the human-derived protein (such as a FGF13 protein). FGF13 orthologs include any mammalian ortholog of FGF13 inclusive of the ortholog in humans and other primates, experimental mammals (such as mice, rats, hamsters and guinea pigs), mammals of commercial significance (such as horses, cows, camels, pigs and sheep), and also companion mammals (such as domestic animals, e.g., rabbits, ferrets, dogs, and cats).

The FGF13 variants can comprise, for instance, naturally-occurring variants due to allelic variations between individuals (e.g., polymorphisms), mutated alleles related to alopecia areata, or alternative splicing forms. In one embodiment, a FGF13 molecule is a nucleic acid variant of the nucleic acid having the sequence shown in SEQ ID NO: 2, 4, 6, 8, 10, or 12, wherein the variant has a nucleotide sequence identity to SEQ ID NO: 2, 4, 6, 8, 10, or 12 of about 65%, about 75%, about 85%, about 90%, about 91%, about 92%, about 93%, about 94%, about 95%, about 96%, about 97%, about 98%, or about 99% with SEQ ID NO: 2, 4, 6, 8, 10, or 1. In one embodiment, a FGF13 molecule encompasses any portion of about 8 consecutive nucleotides of SEQ ID NO: 2, 4, 6, 8, 10, or 1. In one embodiment, the fragment can comprise about 15 nucleotides, about 20 nucleotides, or about 30 nucleotides of SEQ ID NO: 2, 4, 6, 8, 10, or 12. Fragments include all possible nucleotide lengths between about 8 and 100 nucleotides, for example, lengths between about 15 and 100, or between about 20 and 100.

The invention further provides for nucleic acids that are complementary to a nucleic acid encoding a FGF13 protein. Such complementary nucleic acids can comprise nucleic acid sequences, which hybridize to a nucleic acid sequence encoding a FGF13 protein under stringent hybridization conditions. Non-limiting examples of stringent hybridization conditions include temperatures above 30° C., above 35° C., in excess of 42° C., and/or salinity of less than about 500 mM, or less than 200 mM. Hybridization conditions can be adjusted by the skilled artisan via modifying the temperature, salinity and/or the concentration of other reagents such as SDS or SSC.

In one embodiment, a FGF13 molecule comprises a protein or polypeptide encoded by a FGF13 nucleic acid sequence, such as the sequence shown in SEQ ID NO: 1, 3, 5, 7, 9, or 1. In another embodiment, the polypeptide can be modified, such as by glycosylations and/or acetylations and/or chemical reaction or coupling, and can contain one or several non-natural or synthetic amino acids. An example of a FGF13 molecule is the polypeptide having the amino acid sequence shown in SEQ ID NO: 1, 3, 5, 7, 9, or 1. In another embodiment, a FGF13 molecule can be a fragment of a FGF13 protein. For example, the FGF13 molecule can encompass any portion of about 8 consecutive amino acids of SEQ ID NO: 1, 3, 5, 7, 9, or 11. The fragment can comprise about 10 amino acids, a least about 20 amino acids, about 30 amino acids, about 40 amino acids, a least about 50 amino acids, about 60 amino acids, or about 75 amino acids of SEQ ID NO: 1, 3, 5, 7, 9, or 11. Fragments include all possible amino acid lengths between about 8 and 100 about amino acids, for example, lengths between about 10 and 100 amino acids, between about 15 and 100 amino acids, between about 20 and 100 amino acids, between about 35 and 100 amino acids, between about 40 and 100 amino acids, between about 50 and 100 amino acids, between about 70 and 100 amino acids, between about 75 and 100 amino acids, or between about 80 and 100 amino acids.

In certain embodiments, the FGF13 molecule includes variants of the human FGF13 protein (comprising the amino acid sequence shown in SEQ ID NO: 1, 3, 5, 7, 9, or 11). Such variants can include those having at least from about 46% to about 50% identity to SEQ ID NO: 1, 3, 5, 7, 9, or 11, or having at least from about 50.1% to about 55% identity to SEQ ID NO: 1, 3, 5, 7, 9, or 11, or having at least from about 55.1% to about 60% identity to SEQ ID NO: 1, 3, 5, 7, 9, or 11, or having from about 60.1% to about 65% identity to SEQ ID NO: 1, 3, 5, 7, 9, or 11, or having from about 65.1% to about 70% identity to SEQ ID NO: 1, 3, 5, 7, 9, or 11, or having at least from about 70.1% to about 75% identity to SEQ ID NO: 1, 3, 5, 7, 9, or 11, or having at least from about 75.1% to about 80% identity to SEQ ID NO: 1, 3, 5, 7, 9, or 11, or having at least from about 80.1% to about 85% identity to SEQ ID NO: 1, 3, 5, 7, 9, or 11, or having at least from about 85.1% to about 90% identity to SEQ ID NO: 1, 3, 5, 7, 9, or 11, or having at least from about 90.1% to about 95% identity to SEQ ID NO: 1, 3, 5, 7, 9, or 11, or having at least from about 95.1% to about 97% identity to SEQ ID NO: 1, 3, 5, 7, 9, or 11, or having at least from about 97.1% to about 99% identity to SEQ ID NO: 1, 3, 5, 7, 9, or 11.

Techniques for making substitution mutations at predetermined sites in DNA having a known sequence are well known, for example M13 primer mutagenesis and PCR mutagenesis. Amino acid substitutions can be single residues, but can occur at a number of different locations at once. In one non-limiting embodiment, insertions can be on the order of about from 1 to about 10 amino acid residues, while deletions can range from about 1 to about 30 residues. Deletions or insertions can be made in adjacent pairs (for example, a deletion of about 2 residues or insertion of about 2 residues). Substitutions, deletions, insertions, or any combination thereof can be combined to arrive at a final construct. The mutations cannot place the sequence out of reading frame and should not create complementary regions that can produce secondary mRNA structure. Substitutional variants are those in which at least one residue has been removed and a different residue inserted in its place.

Substantial changes in function or immunological identity are made by selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions that can produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Minor variations in the amino acid sequences of proteins are provided by the present invention. The variations in the amino acid sequence can be when the sequence maintains about 30%, about 40%, about 50%, about 60%, about 70%, about 75%, about 80%, about 90%, about 95%, or about 99% identity to SEQ ID NO: 1, 3, 5, 7, 9, or 11. For example, conservative amino acid replacements can be utilized. Conservative replacements are those that take place within a family of amino acids that are related in their side chains, wherein the interchangeability of residues have similar side chains.

Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) a group of amino acids having aliphatic-hydroxyl side chains, such as serine and threonine; (ii) a group of amino acids having amide-containing side chains, such as asparagine and glutamine; (iii) a group of amino acids having aliphatic side chains such as glycine, alanine, valine, leucine, and isoleucine; (iv) a group of amino acids having aromatic side chains, such as phenylalanine, tyrosine, and tryptophan; and (v) a group of amino acids having sulfur-containing side chains, such as cysteine and methionine. Useful conservative amino acids substitution groups are: valine-leucine-isoleucine, phenylalanine-tyrosine, lysine-arginine, alanine valine, glutamic-aspartic, and asparagine-glutamine.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gin; Ser, Thr; Lys, Arg; and Phe, Tyr. Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

In another embodiment, the FGF13 molecule encompasses a peptidomimetic which exhibits FGF13 activity. A peptidomimetic is a small protein-like chain designed to mimic a peptide that can arise from modification of an existing peptide in order to protect that molecule from enzyme degradation and increase its stability, and/or alter the molecule's properties (e.g., modifications that change the molecule's stability or biological activity). These modifications involve changes to the peptide that cannot occur naturally (such as altered backbones and the incorporation of non-natural amino acids). Drug-like compounds can be developed from existing peptides. A peptidomimetic can be a peptide, partial peptide, or non-peptide molecule that mimics the tertiary binding structure or activity of a selected native peptide or protein functional domain (e.g., binding motif or active site). These peptide mimetics include recombinantly or chemically modified peptides.

In one embodiment, a FGF13 molecule comprising SEQ ID NO: 1, 3, 5, 7, 9, or 11, variants of such, or fragments thereof, can be modified to produce peptide mimetics by replacement of one or more naturally occurring side chains of the 20 genetically encoded amino acids (or D amino acids) with other side chains. This can occur, for instance, with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, ifuryl, imidazolidinyl imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics can also have amino acid residues that have been chemically modified by phosphorylation, sulfonation, biotinylation, or the addition or removal of other moieties. For example, peptidomimetics can be designed and directed to amino acid sequences encoded by a FGF13 molecule comprising SEQ ID NO: 1, 3, 5, 7, 9, or 11.

A variety of techniques are available for constructing peptide mimetics with the same or similar desired biological activity as the corresponding native but with more favorable activity than the peptide with respect to solubility, stability, and/or susceptibility to hydrolysis or proteolysis (see, e.g., Morgan & Gainor, Ann. Rep. Med. Chem. 24,243-252, 1989). Certain peptidomimetic compounds are based upon the amino acid sequence of the peptides of the invention. Peptidomimetic compounds can be synthetic compounds having a three-dimensional structure (i.e. a peptide motif) based upon the three-dimensional structure of a selected peptide. The peptide motif provides the peptidomimetic compound with the desired biological activity, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life. Peptidomimetic design strategies are readily available in the art (see, e.g., Ripka & Rich (1998) Curr. Op. Chem. Biol. 2:441-452; Hruby et. al.. (1997) Curr. Op. Chem, Biol. 1:114-119; Hruby & Balse, (2000) Curr. Med. Chem. 9:945-970).

Bacterial and Yeast Expression, Systems.

In bacterial systems, a number of expression vectors can be selected. For example, when a large quantity of a protein encoded by a gene, such as FGF13, is needed for the induction of antibodies, vectors which direct high level expression of proteins that are readily purified can be used. Non-limiting examples of such vectors include multifunctional E. coli cloning and expression vectors such as BLUESCRIPT (Stratagene). pIN vectors or pGEX vectors (Promega, Madison, Wis.) also can be used to express foreign polypeptide molecules as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will.

Plant and Insect Expression Systems.

If plant expression vectors are used, the expression of sequences encoding a FGF13 protein can be driven by any of a number of promoters. For example, viral promoters such as the 35S and 19S promoters of CaMV can be used alone or in combination with the omega leader sequence from TMV. Alternatively, plant promoters such as the small subunit of RUBISCO or heat shock promoters, can be used. These constructs can be introduced into plant cells by direct DNA transformation or by pathogen-mediated transfection.

An insect system also can be used to express the FGF13 protein. For example, in one such system Autographa californica nuclear polyhedrosis virus (AcNPV) is used as a vector to express foreign genes in Spodoptera frugiperda cells or in Trichoplusia larvae. Sequences encoding a polypeptide of FGF13 can be cloned into a non-essential region of the virus, such as the polyhedrin gene, and placed under control of the polyhedrin promoter. Successful insertion of nucleic acid sequences, such as a sequence corresponding to a gene, such as a FGF13 gene, will render the polyhedrin gene inactive and produce recombinant virus lacking coat protein. The recombinant viruses can then be used to infect S. frugiperda cells or Trichoplusia larvae in which the protein or a variant thereof can be expressed.

Mammalian Expression Systems.

An expression vector can include a nucleotide sequence that encodes a FGF13 polypeptide linked to at least one regulatory sequence in a manner allowing expression of the nucleotide sequence in a host cell. A number of viral-based expression systems can be used to express a FGF13 protein or a variant thereof in mammalian host cells. For example, if an adenovirus is used as an expression vector, sequences encoding a protein can be ligated into an adenovirus transcription/translation complex comprising the late promoter and tripartite leader sequence. Insertion into a non-essential E1 or E3 region of the viral genome can be used to obtain a viable virus which expresses a FGF13 protein in infected host cells. Transcription enhancers, such as the Rous sarcoma virus (RSV) enhancer, can also be used to increase expression in mammalian host cells.

Regulatory sequences are well known in the art, and can be selected to direct the expression of a protein or polypeptide of interest in an appropriate host cell as described in Goeddel, Gene Expression Technology: Methods in Enzymology 185, Academic Press, San Diego, Calif. (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Piers, et al., 1973, Nature 273:113; Hager G L, et al., Curr Opin Genet Dev, 2002, 12(2):137-41) enhancers, and other expression control elements.

Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest is stably integrated into the genome of eukaryotic cells (for example mammalian cells, such as cells from the end bulb of the hair follicle), resulting in the stable expression of transfected genes. An exogenous nucleic acid sequence can be introduced into a cell (such as a mammalian cell, either a primary or secondary cell) by homologous recombination as disclosed in U.S. Pat. No. 5,641,670, the contents of which are herein incorporated by reference.

A gene that encodes a selectable marker (for example, resistance to antibiotics or drugs, such as ampicillin, neomycin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduced on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule (for example, a protein encoded by a gene, such as FGF13).

### Cell Transfection

A eukaryotic expression vector can be used to transfect cells in order to produce proteins encoded by nucleotide sequences of the vector. Mammalian cells (such as isolated cells from the hair bulb; for example dermal sheath cells and dermal papilla cells) can contain an expression vector (for example, one that contains a gene encoding a FGF13 protein or polypeptide) via introducing the expression vector into an appropriate host cell via methods known in the art.

A host cell strain can be chosen for its ability to modulate the expression of the inserted sequences or to process the expressed polypeptide encoded by a gene, such as a FGF13 gene, in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and W138), are available from the American Type Culture Collection (ATCC; 10801 University Boulevard, Manassas, Va. 20110-2209) and can be chosen to ensure the correct modification and processing of the foreign protein.

An exogenous nucleic acid can be introduced into a cell via a variety of techniques known in the art, such as lipofection, microinjection, calcium phosphate or calcium chloride precipitation, DEAE-dextran-mediated transfection, or electroporation. Electroporation is carried out at approximate voltage and capacitance to result in entry of the DNA construct(s) into cells of interest (such as cells of the end bulb of a hair follicle, for example dermal papilla cells or dermal sheath cells). Other transfection methods also include modified calcium phosphate precipitation, polybrene precipitation, liposome fusion, and receptor-mediated gene delivery.

Cells that will be genetically engineered can be primary and secondary cells obtained from various tissues, and include cell types which can be maintained and propagated in culture. Non-limiting examples of primary and secondary cells include epithelial cells (for example, dermal papilla cells, hair follicle cells, inner root sheath cells, outer root sheath cells, sebaceous gland cells, epidermal matrix cells), neural cells, endothelial cells, glial cells, fibroblasts, muscle cells (such as myoblasts) keratinocytes, formed elements of the blood (e.g., lymphocytes, bone marrow cells), and precursors of these somatic cell types.

Vertebrate tissue can be obtained by methods known to one skilled in the art, such a punch biopsy or other surgical methods of obtaining a tissue source of the primary cell type of interest. In one embodiment, a punch biopsy or removal can be used to obtain a source of keratinocytes, fibroblasts, endothelial cells, or mesenchymal cells (for example, hair follicle cells or dermal papilla cells). In another embodiment, removal of a hair follicle can be used to obtain a source of fibroblasts, keratinocytes, endothelial cells, or mesenchymal cells (for example, hair follicle cells or dermal papilla cells). A mixture of primary cells can be obtained from the tissue, using methods readily practiced in the art, such as explanting or enzymatic digestion (for examples using enzymes such as pronase, trypsin, collagenase, elastase dispase, and chymotrypsin). Biopsy methods have also been described in U.S. Pat. No. 7,419,661 and PCT application publication WO/2001/032840, and are hereby each incorporated by reference in their entireties.

Primary cells can be acquired from the individual to whom the genetically engineered primary or secondary cells are administered. However, primary cells can also be obtained from a donor, other than the recipient, of the same species. The cells can also be obtained from another species (for example, rabbit, cat, mouse, rat, sheep, goat, dog, horse, cow, bird, or pig). Primary cells can also include cells from an isolated vertebrate tissue source grown attached to a tissue culture substrate (for example, flask or dish) or grown in a suspension; cells present in an explant derived from tissue; both of the aforementioned cell types plated for the first time; and cell culture suspensions derived from these plated cells. Secondary cells can be plated primary cells that are removed from the culture substrate and replated, or passaged, in addition to cells from the subsequent passages. Secondary cells can be passaged one or more times. These primary or secondary cells can contain expression vectors having a gene that encodes a protein of interest (for example, a FGF13 protein or polypeptide).

### Cell Culturing

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized. Non-limiting examples of medium include, for example, Minimal Essential Medium (MEM, Sigma, St. Louis, Mo.); Dulbecco's Modified Eagles Medium (DMEM, Sigma); Ham's F10 Medium (Sigma); HyClone cell culture medium (HyClone, Logan, Utah); RPMI-1640 Medium (Sigma); and chemically-defined (CD) media, which are formulated for various cell types, e.g., CD-CHO Medium (Invitrogen, Carlsbad, Calif.).

The cell culture media can be supplemented as necessary with supplementary components or ingredients, including optional components, in appropriate concentrations or amounts, as necessary or desired. Cell culture medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that can be required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, and epidermal growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example pluronic polyol; and (8) galactose. In one embodiment, soluble factors can be added to the culturing medium.

The mammalian cell culture that can be used with the present invention is prepared in a medium suitable for the type of cell being cultured. In one embodiment, the cell culture medium can be any one of those previously discussed (for example, MEM) that is supplemented with serum from a mammalian source (for example, fetal bovine serum (FBS)). In another embodiment, the medium can be a conditioned medium to sustain the growth of epithelial cells or cells obtained from the hair bulb of a hair follicle (such as dermal papilla cells or dermal sheath cells). For example, epithelial cells can be cultured according to Barnes and Mather in Animal Cell Culture Methods (Academic Press, 1998), which is hereby incorporated by reference in its entirety. In a further embodiment, epithelial cells or hair follicle cells can be transfected with DNA vectors containing genes that encode a polypeptide or protein of interest (for example, a FGF13 protein or polypeptide). In other embodiments of the invention, cells are grown in a suspension culture (for example, a three-dimensional culture such as a hanging drop culture) in the presence of an effective amount of enzyme, wherein the enzyme substrate is an extracellular matrix molecule in the suspension culture. For example, the enzyme can be a hyaluronidase. Epithelial cells or hair follicle cells can be cultivated according to methods practiced in the art, for example, as those described in U.S. Pat. No. 7,785,876, or as described by Harris in Handbook in Practical Animal Cell Biology: Epithelial Cell Culture (Cambridge Univ. Press, Great Britain; 1996; see Chapter 8), which are each hereby incorporated by reference.

A suspension culture is a type of culture wherein cells, or aggregates of cells (such as aggregates of DP cells), multiply while suspended in liquid medium. A suspension culture comprising mammalian cells can be used for the maintenance of cell types that do not adhere or to enable cells to manifest specific cellular characteristics that are not seen in the adherent form. Some types of suspension cultures can include three-dimensional cultures or a hanging drop culture. A hanging-drop culture is a culture in which the material to be cultivated is inoculated into a drop of fluid attached to a flat surface (such as a coverglass, glass slide, Petri dish, flask, and the like), and can be inverted over a hollow surface. Cells in a hanging drop can aggregate toward the hanging center of a drop as a result of gravity. However, according to the methods of the invention, cells cultured in the presence of a protein that degrades the extracellular matrix (such as collagenase, chondroitinase, hyaluronidase, and the like) will become more compact and aggregated within the hanging drop culture, for degradation of the ECM will allow cells to become closer in proximity to one another since less of the ECM will be present. See also U.S. Patent Publication No. US 2010-0303767 A1, which is incorporated by reference.

Cells obtained from the hair bulb of a hair follicle (such as dermal papilla cells or dermal sheath cells) can be cultured as a single, homogenous population (for example, comprising DP cells) in a hanging drop culture so as to generate an aggregate of DP cells. Cells can also be cultured as a heterogeneous population (for example, comprising DP and DS cells) in a hanging drop culture so as to generate a chimeric aggregate of DP and DS cells. Epithelial cells can be cultured as a monolayer to confluency as practiced in the art. Such culturing methods can be carried out essentially according to methods described in Chapter 8 of the Handbook in Practical Animal Cell Biology: Epithelial Cell Culture (Cambridge Univ. Press, Great Britain; 1996); Underhill CB, J Invest Dermatol, 1993, 101(6):820-6); in Armstrong and Armstrong, (1990) J Cell Biol 110:1439-55; or in Animal Cell Culture Methods (Academic Press, 1998), which are all hereby incorporated by reference in their entireties.

Three-dimensional cultures can be formed from agar (such as Gey's Agar), hydrogels (such as matrigel, agarose, and the like; Lee et al., (2004) Biomaterials 25: 2461-2466) or polymers that are cross-linked. These polymers can comprise natural polymers and their derivatives, synthetic polymers and their derivatives, or a combination thereof. Natural polymers can be anionic polymers, cationic polymers, amphipathic polymers, or neutral polymers. Non-limiting examples of anionic polymers can include hyaluronic acid, alginic acid (alginate), carageenan, chondroitin sulfate, dextran sulfate, and pectin. Some examples of cationic polymers, include but are not limited to, chitosan or polylysine. (Peppas et al., (2006) Adv Mater. 18: 1345-60; Hoffman, A. S., (2002) Adv Drug Deliv Rev. 43: 3-12; Hoffman, A. S., (2001) Ann NY Acad Sci 944: 62-73). Examples of amphipathic polymers can include, but are not limited to collagen, gelatin, fibrin, and carboxymethyl chitin. Non-limiting examples of neutral polymers can include dextran, agarose, or pullulan. (Peppas et al., (2006) Adv Mater. 18: 1345-60; Hoffman, A. S., (2002) Adv Drug Deliv Rev. 43: 3-12; Hoffman, A. S., (2001) Ann NY Acad Sei 944: 62-73).

Cells suitable for culturing according to methods of the invention can harbor introduced expression vectors, such as plasmids. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 2001, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

### Obtaining and Purifying Polypeptides

A polypeptide molecule encoded by a gene, such as a FGF13 gene, or a variant thereof, can be obtained by purification from human cells expressing a protein or polypeptide encoded by a FGF13 gene via in vitro or in vivo expression of a nucleic acid sequence encoding a FGF13 protein or polypeptide; or by direct chemical synthesis.

Detecting Polypeptide Expression.

Host cells which contain a nucleic acid encoding a FGF13 protein or polypeptide, and which subsequently express a protein encoded by a FGF13 gene, can be identified by various procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a nucleic acid encoding a FGF13 protein or polypeptide can be detected by DNA-DNA or DNA-RNA hybridization or amplification using probes or fragments of nucleic acids encoding a FGF13 protein or polypeptide. In one embodiment, a fragment of a nucleic acid of a FGF13 gene can encompass any portion of about 8 consecutive nucleotides of SEQ ID NO: 2, 4, 6, 8, 10, or 1. In another embodiment, the fragment can comprise about 10 consecutive nucleotides, about 15 consecutive nucleotides, about 20 consecutive nucleotides, or about 30 consecutive nucleotides of SEQ ID NO: 2, 4, 6, 8, 10, or 12. Fragments can include all possible nucleotide lengths between about 8 and about 100 nucleotides, for example, lengths between about 15 and about 100 nucleotides, or between about 20 and about 100 nucleotides. Nucleic acid amplification-based assays involve the use of oligonucleotides selected from sequences encoding a polypeptide encoded by a FGF13 gene to detect transformants which contain a nucleic acid encoding a FGF13 protein or polypeptide.

Protocols for detecting and measuring the expression of a polypeptide encoded by a gene, such as a FGF13 gene, using either polyclonal or monoclonal antibodies specific for the polypeptide are well established. Non-limiting examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a polypeptide encoded by a gene, such as a FGF13 gene, can be used, or a competitive binding assay can be employed.

Labeling and conjugation techniques are known by those skilled in the art and can be used in various nucleic acid and amino acid assays. Methods for producing labeled hybridization or PCR probes for detecting sequences related to nucleic acid sequences encoding a protein, such as FGF13, include, but are not limited to, oligolabeling, nick translation, end-labeling, or PCR amplification using a labeled nucleotide. Alternatively, nucleic acid sequences encoding a polypeptide encoded by a gene, such as a FGF13 gene, can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, and/or magnetic particles.

Expression and Purification of Polypeptides.

Host cells transformed with a nucleic acid sequence encoding a polypeptide, such as FGF13, can be cultured under conditions suitable for the expression and recovery of the protein from cell culture. The polypeptide produced by a transformed cell can be secreted or contained intracellularly depending on the sequence and/or the vector used. Expression vectors containing a nucleic acid sequence encoding a polypeptide, such as FGF13, can be designed to contain signal sequences which direct secretion of soluble polypeptide molecules encoded by a gene, such as a FGF13 gene, or a variant thereof, through a prokaryotic or eukaryotic cell membrane or which direct the membrane insertion of membrane-bound a polypeptide molecule encoded by a FGF13 gene or a variant thereof.

Other constructions can also be used to join a gene sequence encoding a FGF13 polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein. A domains that allow purification on immobilized immunoglobulin, and the domain utilized in the FLAGS extension/affinity purification system (Immunex Corp., Seattle, Wash.). Including cleavable linker sequences (i.e., those specific for Factor Xa or enterokinase (Invitrogen, San Diego, Calif.)) between the purification domain and a polypeptide encoded by a FGF13 gene also can be used to facilitate purification. One such expression vector provides for expression of a fusion protein containing a polypeptide encoded by a FGF13 gene and 6 histidine residues preceding a thioredoxin or an enterokinase cleavage site. The histidine residues facilitate purification by immobilized metal ion affinity chromatography, while the enterokinase cleavage site provides a means for purifying the polypeptide encoded by a FGF13 gene.

A FGF13 polypeptide can be purified from any human or non-human cell which expresses the polypeptide, including those which have been transfected with expression constructs that express a FGF13 protein. A purified FGF13 protein can be separated from other compounds which normally associate with a protein encoded by a FGF13 gene in the cell, such as certain proteins, carbohydrates, or lipids, using methods practiced in the art. Non-limiting methods include size exclusion chromatography, ammonium sulfate fractionation, ion exchange chromatography, affinity chromatography, and preparative gel electrophoresis.

Chemical Synthesis.

Nucleic acid sequences comprising a gene, such as a FGF13 gene, that encodes a polypeptide can be synthesized, in whole or in part, using chemical methods known in the art. Alternatively, a polypeptide, such as FGF13, can be produced using chemical methods to synthesize its amino acid sequence, such as by direct peptide synthesis using solid-phase techniques. Protein synthesis can either be performed using manual techniques or by automation. Automated synthesis can be achieved, for example, using Applied Biosystems 431A Peptide Synthesizer (Perkin Elmer). Optionally, fragments of FGF13 polypeptides can be separately synthesized and combined using chemical methods to produce a full-length molecule. In one embodiment, a fragment of a nucleic acid sequence that comprises a FGF13 gene can encompass any portion of about 8 consecutive nucleotides of SEQ ID NO: 2, 4, 6, 8, 10, or 12. In one embodiment, the fragment can comprise about 10 nucleotides, about 15 nucleotides, about 20 nucleotides, or about 30 nucleotides of SEQ ID NO: 2, 4, 6, 8, 10, or 12. Fragments include all possible nucleotide lengths between about 8 and about 100 nucleotides, for example, lengths between about 15 and about 100 nucleotides, or between about 20 and about 100 nucleotides.

A FGF13 fragment can be a fragment of a protein, such as FGF13. For example, the FGF13 fragment can encompass any portion of about 8 consecutive amino acids of SEQ ID NO: 1, 3, 5, 7, 9, or 11. The fragment can comprise about 10 consecutive amino acids, about 20 consecutive amino acids, about 30 consecutive amino acids, about 40 consecutive amino acids, a least about 50 consecutive amino acids, about 60 consecutive amino acids, about 70 consecutive amino acids, or about 75 consecutive amino acids of SEQ ID NO: 1, 3, 5, 7, 9, or 11. Fragments include all possible amino acid lengths between about 8 and 100 about amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids.

A synthetic peptide can be substantially purified via high performance liquid chromatography (HPLC). The composition of a synthetic polypeptide of FGF13 can be confirmed by amino acid analysis or sequencing. Additionally, any portion of an amino acid sequence comprising a protein encoded by a FGF13 gene can be altered during direct synthesis and/or combined using chemical methods with sequences from other proteins to produce a variant polypeptide or a fusion protein.

### Identifying FGF13 Modulating Compounds

The invention provides methods for identifying compounds which can be used for controlling and/or regulating hair growth (for example, hair density) in a subject. Since the invention has provided the identification of the gene listed herein as a gene associated with a hair loss disorder, the invention also provides methods for identifying compounds that modulate the expression or activity of a gene and/or protein of FGF1. In addition, the invention provides methods for identifying compounds which can be used for the treatment of a hair loss disorder. The invention also provides methods for identifying compounds which can be used for the treatment of hypotrichosis (for example, hereditary hypotrichosis simplex (HHS)), Non limiting examples of hair loss disorders include: androgenetic alopecia, Alopecia areata, telogen effluvium, alopecia areata, alopecia totalis, and alopecia universalis. The invention also provides methods for identifying compounds which can be used for the treatment of a hair growth disorder. The invention also provides methods for identifying compounds which can be used for the treatment of hypertrichosis (for example, X-linked hypertrichosis). Non-limiting examples of hair growth disorders include X-linked hypertrichosis, generalized hypertrichosis terminalis with or without gingival hyperplasia, autosomal recessive hypertrichosis, Cantu syndrome, Ambras type hypertrichosis and autosomal recessive trichomegaly. The methods can comprise the identification of test compounds or agents (e.g., peptides (such as antibodies or fragments thereof), small molecules, nucleic acids (such as siRNA or antisense RNA), or other agents) that can bind to a polypeptide molecule encoded by a FGF13 gene and/or have a stimulatory or inhibitory effect on the biological activity of a protein encoded by a FGF13 gene or its expression, and subsequently determining whether these compounds can regulate hair growth in a subject or can have an effect on symptoms associated with the hair loss disorders or hair growth disorders in an in vivo assay (i.e., examining an increase or reduction in hair growth).

As used herein, a “FGF13 modulating compound” refers to a compound that interacts with a FGF13 gene or a FGF13 protein or polypeptide and modulates its activity and/or its expression. The compound can either increase the activity or expression of a protein encoded by a FGF13 gene. The compound can be a FGF13 agonist (e.g., a FGF13 activator). In one embodiment, the FGF13 activator is a polypeptide comprising SEQ ID NO: 1, 3, 5, 7, 9, or 11, or a fragment thereof; or a peptidomimetic comprising SEQ ID NO: 1, 3, 5, 7, 9, or 11. Conversely, the compound can decrease the activity or expression of a protein encoded by a FGF13 gene. The compound can be a FGF13 agonist or a FGF13 antagonist (e.g., a FGF13 inhibitor). Some non-limiting examples of FGF13 modulating compounds include peptides (such as peptide fragments comprising a polypeptide encoded by a FGF13 gene, or antibodies or fragments thereof), small molecules, and nucleic acids (such as siRNA or antisense RNA specific for a nucleic acid comprising a FGF13 gene). Agonists of a FGF13 protein can be molecules which, when bound to a FGF13 protein, increase or prolong the activity of the FGF13 protein. FGF13 agonists include, but are not limited to, proteins, nucleic acids, small molecules, or any other molecule which activates a FGF13 protein. Antagonists of a FGF13 protein can be molecules which, when bound to a FGF13 protein decrease the amount or the duration of the activity of the FGF13 protein. Antagonists include proteins, nucleic acids, antibodies, small molecules, or any other molecule which decrease the activity of a FGF13 protein.

The term “modulate,” as it appears herein, refers to a change in the activity or expression of a gene or protein of FGF13. For example, modulation can cause an increase or a decrease in protein activity, binding characteristics, or any other biological, functional, or immunological properties of a FGF13 protein.

In one embodiment, a FGF13 modulating compound can be a peptide fragment of a FGF13 protein that binds to the protein. For example, the FGF13 polypeptide can encompass any portion of about 8 consecutive amino acids of SEQ ID NO: 1, 3, 5, 7, 9, or 11. The fragment can comprise about 10 consecutive amino acids, about 20 consecutive amino acids, about 30 consecutive amino acids, about 40 consecutive amino acids, about 50 consecutive amino acids, about 60 consecutive amino acids, or about 75 consecutive amino acids of SEQ ID NO: 1, 3, 5, 7, 9, or 11. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England). The FGF13 peptide fragments can be isolated from a natural source, genetically engineered, or chemically prepared. These methods are well known in the art.

A FGF13 modulating compound can be a protein, such as an antibody (monoclonal, polyclonal, humanized, chimeric, or fully human), or a binding fragment thereof, directed against a polypeptide encoded by a FGF13 gene. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (see Roland E. Kontermann and Stefan Dübel (editors), Antibody Engineering, Vol. I & II, (2010) 2^{nd} ed., Springer; Antony S. Dimitrov (editor), Therapeutic Antibodies: Methods and Protocols (Methods in Molecular Biology), (2009), Humana Press; Benny Lo (editor) Antibody Engineering: Methods and Protocols (Methods in Molecular Biology), (2004) Humana Press, each of which are hereby incorporated by reference in their entireties). For example, antibodies directed to FGF13 can be obtained commercially from Abeam, Santa Cruz Biotechnology, Abgent, R&D Systems, Novus Biologicals, etc. Human antibodies directed to either FGF13 (such as monoclonal, humanized, or chimeric antibodies) can be useful antibody therapeutics for use in humans. In one embodiment, an antibody or binding fragment thereof is directed against SEQ ID NO: 1, 3, 5, 7, 9, or 11.

Inhibition of RNA encoding a polypeptide encoded by a FGF13 gene can effectively modulate the expression of a FGF13 gene from which the RNA is transcribed. Inhibitors are selected from the group comprising: siRNA; interfering RNA or RNAi; dsRNA; RNA Polymerase III transcribed DNAs; ribozymes; and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. For example, antisense oligonucleotides of about 15 bases and complementary to unique regions of the DNA sequence encoding a polypeptide encoded by a FGF13 gene can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59). Antisense nucleotide sequences include, but are not limited to: morpholinos, 2′-O-methyl polynucleotides, DNA, RNA and the like. In one embodiment, the FGF13 antisense oligonucleotide comprises CACCACCACCGCTTCTTTTGCTGC (SEQ ID NO: 21).

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area. See also, McMnaus and Sharp (2002) Nat Rev Genetics, 3:737-47, and Sen and Blau (2006) FASEB J. 20:1293-99, the entire disclosures of which are herein incorporated by reference.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribo-nucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector (for example, see U.S. Pat. No. 7,294,504 and U.S. Pat. No. 7,422,896, the entire disclosures of which are herein incorporated by reference). Exemplary methods for producing and testing dsRNA or siRNA molecules are described in U.S. Patent Application Publication No. 2002/0173478 to Gewirtz, U.S. Pat. No. 8,071,559 to Hannon et al., U.S. Pat. No. 7,674,895 to Reich et al., and in U.S. Pat. No. 7,148,342 to Tolentino et al., the entire disclosures of which each are hereby incorporated by reference.

In one embodiment, an siRNA directed to a human nucleic acid sequence comprising a FGF13 gene can be generated against SEQ ID NO: 2, 4, 6, 8, 10, or 1. In another embodiment, an siRNA directed to a human nucleic acid sequence comprising a FGF13 gene can comprise any one of the sequences listed in Table 1.

In another embodiment, the siRNA directed to FGF13 is listed in Table 1.

TABLE 1
siRNA SEQUENCES for FGF13

| SEQ ID NO: | |
|---|---|
| 13 | GAACAAAGAAGGAGAGATC |
| 14 | CAGCTTAAGGGTATAGTTA |
| 15 | ACAAAGAAGGAGAGATCAT |
| 16 | GCAACCATGTGAAGAAGAA |
| 17 | GAACAAGCCTGCAGCTCAT |
| 18 | GCACTTACACTCTGTTTAA |
| 19 | GAGAGATCATGAAAGGCAA |
| 20 | TGAAAGTGGCCATGTACAA |

RNA polymerase III transcribed DNAs contain promoters, such as the U6 promoter. These DNAs can be transcribed to produce small hairpin RNAs in the cell that can function as siRNA or linear RNAs that can function as antisense RNA. The FGF13 modulating compound can contain ribonucleotides, deoxyribonucleotides, synthetic nucleotides, or any suitable combination such that the target RNA and/or gene is inhibited. In addition, these forms of nucleic acid can be single, double, triple, or quadruple stranded. (see for example Bass (2001) Nature, 411:428-429; Elbashir et al., (2001) Nature, 411:494 498; U.S. Pat. No. 6,509,154; and PCT Publication Nos. WO 00/044895, WO 01/036646, WO 99/032619, WO 00/01846, WO 01/029058, WO 00/44914).

A FGF13 modulating compound can be a small molecule that binds to a FGF13 protein and disrupts its function, or conversely, enhances its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate a FGF13 protein can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described below (Werner et al., (2006) Brief Funct. Genomic Proteomic 5(1):32-6).

Knowledge of the primary sequence of a molecule of interest, such as a polypeptide encoded by a FGF13 gene, and the similarity of that sequence with proteins of known function, can provide information as to the inhibitors or antagonists of the protein of interest in addition to agonists. Identification and screening of agonists and antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Test compounds, such as FGF13 modulating compounds, can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76). Numerous means are currently used for random and directed synthesis of saccharide, peptide, and nucleic acid based compounds. Synthetic compound libraries are commercially available from Maybridge Chemical Co. (Trevillet, Cornwall, UK), AMRI (Albany, N.Y.), ChemBridge (San Diego, Calif.), and MicroSource (Gaylordsville, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. For example, libraries can also include, but are not limited to, peptide-on-plasmid libraries, synthetic small molecule libraries, aptamer libraries, in vitro translation-based libraries, polysome libraries, synthetic peptide libraries, neurotransmitter libraries, and chemical libraries.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/020242, dated Oct. 14, 1993; and Brenner et al., (1992) Proc. Natl. Acad. Sci. USA 89:5381-5383.

Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/108318.

In vitro translation-based libraries include but are not limited to those described in PCT Publication No. WO 91/005058; and Mattheakis et al., (1994) Proc. Natl. Acad. Sci. USA 91:9022-9026.

As used herein, the term “ligand source” can be any compound library described herein, or tissue extract prepared from various organs in an organism's system, that can be used to screen for compounds that would act as an agonist or antagonist of a FGF13 protein. Screening compound libraries listed herein (also see U.S. Pat. No. 7,884,189, which is hereby incorporated by reference in its entirety], in combination with in vivo animal studies, functional and signaling assays described below can be used to identify FGF13 modulating compounds that regulate hair growth or treat hair loss disorders.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815, 5,223,409, and 5,198,346, all to Ladner et al.; Rebar et al., (1993) Science 263:671-673; and PCT Publication No. WO 94/018318.

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT Publication No. WO 95/018972, as well as U.S. Pat. No. 5,712,171 and its corresponding PCT Publication No. WO 96/022529, which are each hereby incorporated by reference in their entireties.

In one non-limiting example, non-peptide libraries, such as a benzodiazepine library (see e.g., Bunin et al., (1994) Proc. Natl. Acad. Sci. USA 91:4708-4712), can be screened. Peptoid libraries, such as that described by Simon et al., (1992) Proc. Natl. Acad. Sci. USA 89:9367-9371, can also be used. Another example of a library that can be used, in which the amide functionalities in peptides have been pennethylated to generate a chemically transformed combinatorial library, is described by Ostresh et al. (1994), Proc. Natl. Acad. Sci. USA 91:11138-11142.

Computer modeling and searching technologies permit the identification of compounds, or the improvement of already identified compounds, that can modulate the expression or activity of a FGF13 protein. Having identified such a compound or composition, the active sites or regions of a FGF13 protein can be subsequently identified via examining the sites to which the compounds bind. These sites can be ligand binding sites and can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

The three dimensional geometric structure of a site, for example that of a polypeptide encoded by a FGF13 gene, can be determined by known methods in the art, such as X-ray crystallography, which can determine a complete molecular structure. Solid or liquid phase NMR can be used to determine certain intramolecular distances. Any other experimental method of structure determination can be used to obtain partial or complete geometric structures. The geometric structures can be measured with a complexed ligand, natural or artificial, which can increase the accuracy of the active site structure determined.

Other methods for preparing or identifying peptides that bind to a target are known in the art. Molecular imprinting, for instance, can be used for the de novo construction of macromolecular structures such as peptides that bind to a molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Mosbach, (1994) Trends in Biochem. Sci., 19(9); and Wulff, G., in Polymeric Reagents and Catalysts (Ford, W. T., Ed.) ACS Symposium Series No. 308, pp 186-230, American Chemical Society (1986). One method for preparing mimics of a FGF13 modulating compound involves the steps of: (i) polymerization of functional monomers around a known substrate (the template) that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are prepared by the free radical polymerization of functional monomers, resulting in a compound with a nonbiodegradable backbone. Other methods for designing such molecules include for example drug design based on structure activity relationships, which require the synthesis and evaluation of a number of compounds and molecular modeling.

### Screening Assays

FGF13 Modulating Compounds.

A FGF13 modulating compound can be a compound that affects the activity and/or expression of a FGF13 protein in vivo and/or in vitro. FGF13 modulating compounds can be agonists and antagonists of a FGF13 protein, and can be compounds that exert their effect on the activity of a FGF13 protein via the expression, via post-translational modifications, or by other means.

Test compounds or agents which bind to a FGF13 protein, and/or have a stimulatory or inhibitory effect on the activity or the expression of a FGF13 protein, can be identified by two types of assays: (a) cell-based assays which utilize cells expressing a FGF13 protein or a variant thereof on the cell surface; or (b) cell-free assays, which can make use of isolated FGF13 proteins. These assays can employ a biologically active fragment of a FGF13 protein, full-length proteins, or a fusion protein which includes all or a portion of a polypeptide encoded by a FGF13 gene. A FGF13 protein can be obtained from any suitable mammalian species (e.g., human, rat, chick, xenopus, equine, bovine or murine). The assay can be a binding assay comprising direct or indirect measurement of the binding of a test compound. The assay can also be an activity assay comprising direct or indirect measurement of the activity of a FGF13 protein. The assay can also be an expression assay comprising direct or indirect measurement of the expression of FGF13 mRNA nucleic acid sequences or a protein encoded by a FGF13 gene. The various screening assays can be combined with an in vivo assay comprising measuring the effect of the test compound on the symptoms of a hair loss disorder or disease in a subject (for example, androgenetic alopecia, alopecia areata, alopecia totalis, or alopecia universalis), hair growth disorder (for example, hypertrichosis), or even hypotrichosis.

An in viva assay can also comprise assessing the effect of a test compound on regulating hair growth in known mammalian models that display defective or aberrant hair growth phenotypes or mammals that contain mutations in the open reading frame (ORF) of nucleic acid sequences comprising a FGF13 gene that affects hair growth regulation or hair density. In one embodiment, controlling hair growth can comprise an induction of hair growth or density in the subject. Here, the compound's effect in regulating hair growth can be observed either visually via examining the organism's physical hair growth or loss, or by assessing protein or mRNA expression using methods known in the art.

Assays for screening test compounds that bind to or modulate the activity of a FGF13 protein can also be carried out. The test compound can be obtained by any suitable means, such as from conventional compound libraries. Determining the ability of the test compound to bind to a membrane-bound form of the FGF13 protein can be accomplished via coupling the test compound with a radioisotope or enzymatic label such that binding of the test compound to the cell expressing a FGF13 protein can be measured by detecting the labeled compound in a complex. For example, the test compound can be labeled with ³H, ¹⁴C, ³⁵S, or ¹²⁵I, either directly or indirectly, and the radioisotope can be subsequently detected by direct counting of radioemmission or by scintillation counting. Alternatively, the test compound can be enzymatically labeled with, for example, horseradish peroxidase, alkaline phosphatase, or luciferase, and the enzymatic label detected by determination of conversion of an appropriate substrate to product.

Cell-based assays can comprise contacting a cell expressing FGF13 with a test agent and determining the ability of the test agent to modulate (such as increase or decrease) the activity or the expression of the membrane-bound molecule. Determining the ability of the test agent to modulate the activity of the membrane-bound FGF13 molecule can be accomplished by any method suitable for measuring the activity of such a molecule, such as monitoring downstream signaling events (e.g., You et al., Ann N Y Acad Sci. 2008 December; 1150:300-10; Posadas et al., Expert Rev Clin Immunol. 2009 January; 5(1):9-17; Korhonen et al., Basic Clin Pharmacol Toxicol. 2009 April; 104(4):276-84; Vital et al., Ther Clin Risk Manag. 2006 December; 2(4):365-75; Malek and Castro, Immunity. 2010 Aug. 27; 33(2):153-65; Cheng et al., Immunol Rev. 2011 May; 241(1):63-76; Lanier, Nat Immunol. 2008 May; 9(5):495-502; Lowell, Cold Spring Harb Perspect Biol. 2011 Mar. 1; 3(3). pii: a002352; Mócsai et al., Nat Rev Immunol. 2010 June; 10(6):387-402; Bradshaw, Cell Signal. 2010 August; 22(8):1175-84; Ivanenkov et al., Mini Rev Med Chem. 2011 January; 11(1):55-78; Himpe et al., Biofactors. 2009 January-February; 35(1):76-81, each of which are incorporated by reference in their entireties).

A FGF13 protein or the target of a FGF13 protein can be immobilized to facilitate the separation of complexed from uncomplexed forms of one or both of the proteins. Binding of a test compound to a FGF13 protein or a variant thereof, or interaction of a FGF13 protein with a target molecule in the presence and absence of a test compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix (for example, glutathione-S-transferase (GST) fusion proteins or glutathione-S-transferase fusion proteins can be adsorbed onto glutathione sepharose beads (Sigma Chemical; St. Louis, Mo.) or glutathione derivatized microtiter plates).

A FGF13 protein, or a variant thereof, can also be immobilized via being bound to a solid support. Non-limiting examples of suitable solid supports include glass or plastic slides, tissue culture plates, microtiter wells, tubes, silicon chips, or particles such as beads (including, but not limited to, latex, polystyrene, or glass beads). Any method known in the art can be used to attach a polypeptide (or polynucleotide) corresponding to FGF13 or a variant thereof, or test compound to a solid support, including use of covalent and non-covalent linkages, or passive absorption.

The expression of a FGF13 protein can also be monitored. For example, regulators of the expression of a FGF13 protein can be identified via contacting a cell with a test compound and determining the expression of a protein encoded by a FGF13 gene or FGF13 mRNA nucleic acid sequences in the cell. The expression level of a protein encoded by a FGF13 gene or FGF13 mRNA nucleic acid sequences in the cell in the presence of the test compound is compared to the protein or mRNA expression level in the absence of the test compound. The test compound can then be identified as a regulator of the expression of a FGF13 protein based on this comparison. For example, when expression of a protein encoded by a FGF13 gene or FGF13 mRNA nucleic acid sequences in the cell is statistically or significantly greater in the presence of the test compound than in its absence, the test compound is identified as a stimulator/enhancer of expression of a protein encoded by a FGF13 gene or FGF13 mRNA nucleic acid sequences in the cell. The test compound can be said to be a FGF13 modulating compound (such as an agonist).

Alternatively, when expression of a protein encoded by a FGF13 gene or FGF13 mRNA nucleic acid sequences in the cell is statistically or significantly less in the presence of the test compound than in its absence, the compound is identified as an inhibitor of the expression of a protein encoded by a FGF13 gene or FGF13 mRNA nucleic acid sequences in the cell. The test compound can also be said to be a FGF13 modulating compound (such as an antagonist). The expression level of a protein encoded by a FGF13 gene or FGF13 mRNA nucleic acid sequences in the cell in cells can be determined by methods previously described.

For binding assays, the test compound can be a small molecule which binds to and occupies the binding site of a polypeptide encoded by a FGF13 gene, or a variant thereof. This can make the ligand binding site inaccessible to substrate such that normal biological activity is prevented. Examples of such small molecules include, but are not limited to, small peptides or peptide-like molecules. In binding assays, either the test compound or a polypeptide encoded by a FGF13 gene can comprise a detectable label, such as a fluorescent, radioisotopic, chemiluminescent, or enzymatic label (for example, alkaline phosphatase, horseradish peroxidase, or luciferase). Detection of a test compound which is bound to a polypeptide encoded by a FGF13 gene can then be determined via direct counting of radioemmission, by scintillation counting, or by determining conversion of an appropriate substrate to a detectable product.

Determining the ability of a test compound to bind to a FGF13 protein also can be accomplished using real-time Biamolecular Interaction Analysis (BIA) [McConnell et al., 1992, Science 257, 1906-1912; Sjolander, Urbaniczky, 1991, Anal. Chem. 63, 2338-23451. BIA is a technology for studying biospecific interactions in real time, without labeling any of the interactants (for example, BIA-core™). Changes in the optical phenomenon surface plasmon resonance (SPR) can be used as an indication of real-time reactions between biological molecules.

To identify other proteins which bind to or interact with a FGF13 protein and modulate its activity, a polypeptide encoded by a FGF13 gene can be used as a bait protein in a two-hybrid assay or three-hybrid assay (Szabo et al., 1995, Curr. Opin. Struct. Biol. 5, 699-705; U.S. Pat. No. 5,283,317), according to methods practiced in the art. The two-hybrid system is based on the modular nature of most transcription factors, which consist of separable DNA-binding and activation domains.

Functional Assays.

Test compounds can be tested for the ability to increase or decrease the activity of a FGF13 protein, or a variant thereof. Activity can be measured after contacting a purified FGF13 protein, a cell membrane preparation, or an intact cell with a test compound. A test compound that decreases the activity of a FGF13 protein by about 10%, about 20%, about 30%, about 40%, about 50%, about 60%, about 70%, about 75%, about 80%, about 90%, about 95% or 100% is identified as a potential agent for decreasing the activity of a FGF13 protein, for example an antagonist. A test compound that increases the activity of a FGF13 protein by about 10%, about 20%, about 30%, about 40%, about 50%, about 60%, about 70%, about 75%, about 80%, about 90%, about 95% or 100% is identified as a potential agent for increasing the activity of a FGF13 protein, for example an agonist.

### Treatment and Prevention

The invention also provides a method for treating or preventing a hair-loss disorder in a subject. In one embodiment, the method comprises detecting the presence of an alteration in a FGF13 gene in a sample from the subject, the presence of the alteration being indicative of a hair-loss disorder, or the predisposition to a hair-loss disorder, and, administering to the subject in need a therapeutic treatment against a hair-loss disorder. The therapeutic treatment can be a drug administration (for example, a pharmaceutical composition comprising a siRNA directed to a FGF13 nucleic acid). In one embodiment, the therapeutic molecule to be administered comprises a polypeptide encoded by a FGF13 gene, comprising about 75%, about 80%, about 85%, about 90%, about 93%, about 95%, about 97%, about 98%, about 99%, or 100% of the amino acid sequence of SEQ ID NO: 1, 3, 5, 7, 9, or 11, and exhibits the function of decreasing expression of a protein encoded by a FGF13 gene. This can restore the capacity to initiate hair growth in cells derived from hair follicles or skin. In another embodiment, the therapeutic molecule to be administered comprises a nucleic acid sequence comprising a FGF13 gene that encodes a polypeptide, comprising about 75%, about 80%, about 85%, about 90%, about 93%, about 95%, about 97%, about 98%, about 99%, or 100% of the nucleic acid sequence of SEQ ID NO: 2, 4, 6, 8, 10, or 12, and encodes a polypeptide with the function of decreasing expression of a protein encoded by a FGF13 gene, thus restoring the capacity to initiate hair growth in cells derived from hair follicle cells or skin.

The alteration can be determined at the level of the DNA, RNA, or polypeptide. Optionally, detection can be determined by performing an oligonucleotide ligation assay, a confirmation based assay, a hybridization assay, a sequencing assay, an allele-specific amplification assay, a microsequencing assay, a melting curve analysis, a denaturing high performance liquid chromatography (DHPLC) assay (for example, see Jones et al, (2000) Hum Genet., 106(6):663-8), or a combination thereof. In another embodiment, the detection is performed by sequencing all or part of a FGF13 gene or by selective hybridization or amplification of all or part of a FGF13 gene. A FGF13 gene specific amplification can be carried out before the alteration identification step.

An alteration in a chromosome region occupied by a FGF13 gene can be any form of mutation(s), deletion(s), rearrangement(s) and/or insertions in the coding and/or non-coding region of the locus, alone or in various combination(s). Mutations can include point mutations. Insertions can encompass the addition of one or several residues in a coding or non-coding portion of the gene locus. Insertions can comprise an addition of between 1 and 50 base pairs in the gene locus. Deletions can encompass any region of one, two or more residues in a coding or non-coding portion of the gene locus, such as from two residues up to the entire gene or locus. Deletions can affect smaller regions, such as domains (introns) or repeated sequences or fragments of less than about 50 consecutive base pairs, although larger deletions can occur as well. Rearrangement includes inversion of sequences. The alteration in a chromosome region occupied by a FGF13 gene can result in amino acid substitutions, RNA splicing or processing, product instability, the creation of stop codons, frame-shift mutations, and/or truncated polypeptide production. The alteration can result in the production of a polypeptide encoded by a FGF13 gene with altered function, stability, targeting or structure. The alteration can also cause a reduction, or even an increase in protein expression. In one embodiment, the alteration in the chromosome region occupied by a FGF13 gene can comprise a point mutation, a deletion, or an insertion in a FGF13 gene or corresponding expression product. In another embodiment, the alteration can be a deletion or partial deletion of a FGF13 gene. The alteration can be determined at the level of the DNA, RNA, or polypeptide.

In another embodiment, the method can comprise detecting the presence of altered RNA expression. Altered RNA expression includes the presence of an altered RNA sequence, the presence of an altered RNA splicing or processing, or the presence of an altered quantity of RNA. These can be detected by various techniques known in the art, including sequencing all or part of the RNA or by selective hybridization or selective amplification of all or part of the RNA. In a further embodiment, the method can comprise detecting the presence of altered expression of a polypeptide encoded by a FGF13 gene. Altered polypeptide expression includes the presence of an altered polypeptide sequence, the presence of an altered quantity of polypeptide, or the presence of an altered tissue distribution. These can be detected by various techniques known in the art, including by sequencing and/or binding to specific ligands (such as antibodies).

Various techniques known in the art can be used to detect or quantify altered gene or RNA expression or nucleic acid sequences, which include, but are not limited to, hybridization, sequencing, amplification, and/or binding to specific ligands (such as antibodies). Other suitable methods include allele-specific oligonucleotide (ASO), oligonucleotide ligation, allele-specific amplification, Southern blot (for DNAs), Northern blot (for RNAs), single-stranded conformation analysis (SSCA),), pulsed-field gel electrophoresis (PFGE), isoelectric focusing, fluorescent in situ hybridization (FISH), gel migration, clamped denaturing gel electrophoresis, denaturing HPLC, melting curve analysis, heteroduplex analysis, RNase protection, chemical or enzymatic mismatch cleavage, ELISA, radio-immunoassays (RIA) and immuno-enzymatic assays (IEMA). In one embodiment, the detecting comprises using a northern blot; real time PCR and primers directed to SEQ ID NO: 2, 4, 6, 8, 10, or 12; a ribonuclease protection assay; a hybridization, amplification, or sequencing technique to distinguish SEQ ID NO: 2, 4, 6, 8, 10, or 12; or a combination thereof.

Some of these approaches (such as SSCA and constant gradient gel electrophoresis (CGGE)) are based on a change in electrophoretic mobility of the nucleic acids, as a result of the presence of an altered sequence. According to these techniques, the altered sequence is visualized by a shift in mobility on gels. The fragments can then be sequenced to confirm the alteration. Some other approaches are based on specific hybridization between nucleic acids from the subject and a probe specific for wild type or altered gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand specific for the polypeptide, for example, the use of a specific antibody.

Sequencing.

Sequencing can be carried out using techniques well known in the art, using automatic sequencers. The sequencing can be performed on the complete FGF13 gene or on specific domains thereof, such as those known or suspected to carry deleterious mutations or other alterations.

Amplification.

Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR, allele-specific PCR, or PCR based single-strand conformational polymorphism (SSCP). Amplification usually requires the use of specific nucleic acid primers, to initiate the reaction. Nucleic acid primers useful for amplifying sequences from a FGF13 gene or locus are able to specifically hybridize with a portion of a FGF13 gene locus that flank a target region of the locus, wherein the target region is altered in certain subjects having a hair-loss disorder. In one embodiment, amplification can comprise using forward and reverse PCR primers comprising nucleotide sequences of SEQ ID NO: 2, 4, 6, 8, 10, or 12. Non-limiting amplification methods include, e.g., polymerase chain reaction, PCR (PCR Protocols, A Guide To Methods And Applications, ed. Innis, Academic Press, N.Y., 1990 and PCR Strategies, 1995, ed. Innis, Academic Press, Inc., N.Y.); ligase chain reaction (LCR) (Wu (1989) Genomics 4:560; Landegren (1988) Science 241:1077; Barringer (1990) Gene 89:117); transcription amplification (Kwoh (1989) PNAS 86:1173); and, self-sustained sequence replication (Guatelli (1990) PNAS 87:1874); Q Beta replicase amplification (Smith (1997) J. Clin. Microbiol. 35:1477-1491), automated Q-beta replicase amplification assay (Burg (1996) Mol. Cell. Probes 10:257-271) and other RNA polymerase mediated techniques (e.g., NASBA, Cangene, Mississauga, Ontario; see also Berger (1987) Methods Enzymol. 152:307-316; U.S. Pat. Nos. 4,683,195 and 4,683,202; and Sooknanan (1995) Biotechnology 13:563-564). All the references stated above are incorporated by reference in their entireties.

The invention provides for a nucleic acid primer, wherein the primer can be complementary to and hybridize specifically to a portion of a FGF13 coding sequence (e.g., gene or RNA) altered in certain subjects having a hair-loss disorder. Primers of the invention can be specific for altered sequences in a FGF13 gene or RNA. By using such primers, the detection of an amplification product indicates the presence of an alteration in a FGF13 gene or the absence of such gene. Primers can also be used to identify single nucleotide polymorphisms (SNPs) located in or around a FGF13 gene locus; SNPs can comprise a single nucleotide change, or a cluster of SNPs in and around a FGF13 gene. Examples of primers of this invention can be single-stranded nucleic acid molecules of about 5 to 60 nucleotides in length, or about 8 to about 25 nucleotides in length. The sequence can be derived directly from the sequence of a FGF13 gene. Perfect complementarity is useful to ensure high specificity; however, certain mismatch can be tolerated. For example, a nucleic acid primer or a pair of nucleic acid primers as described above can be used in a method for detecting the presence of or a predisposition to a hair-loss disorder in a subject.

Selective Hybridization.

Hybridization detection methods are based on the formation of specific hybrids between complementary nucleic acid sequences that serve to detect nucleic acid sequence alteration(s). A detection technique involves the use of a nucleic acid probe specific for wild type or altered gene or RNA, followed by the detection of the presence of a hybrid. The probe can be in suspension or immobilized on a substrate or support (for example, as in nucleic acid array or chips technologies). The probe can be labeled to facilitate detection of hybrids. For example, a sample from the subject can be contacted with a nucleic acid probe specific for a wild type FGF13 gene or an altered FGF13 gene, and the formation of a hybrid can be subsequently assessed. In one embodiment, the method comprises contacting simultaneously the sample with a set of probes that are specific, respectively, for a wild type FGF13 gene and for various altered forms thereof. Thus, it is possible to detect directly the presence of various forms of alterations in a FGF13 gene in the sample. Also, various samples from various subjects can be treated in parallel.

According to the invention, a probe can be a polynucleotide sequence which is complementary to and can specifically hybridize with a (target portion of a) FGF13 gene or RNA, and that is suitable for detecting polynucleotide polymorphisms associated with alleles of a FGF13 gene (or genes) which predispose to or are associated with a hair-loss disorder. Useful probes are those that are complementary to a FGF13 gene, RNA, or target portion thereof. Probes can comprise single-stranded nucleic acids of between 8 to 1000 nucleotides in length, for instance between 10 and 800, between 15 and 700, or between 20 and 500. Longer probes can be used as well. A useful probe of the invention is a single stranded nucleic acid molecule of between 8 to 500 nucleotides in length, which can specifically hybridize to a region of a FGF13 gene or RNA that carries an alteration. For example, the probe can be directed to a chromosome region occupied by a FGF13 gene.

The sequence of the probes can be derived from the sequences of a FGF13 gene and RNA as provided herein. Nucleotide substitutions can be performed, as well as chemical modifications of the probe. Such chemical modifications can be accomplished to increase the stability of hybrids (e.g., intercalating groups) or to label the probe. Some examples of labels include, without limitation, radioactivity, fluorescence, luminescence, and enzymatic labeling.

A guide to the hybridization of nucleic acids is found in e.g., Sambrook, ed., Molecular Cloning: A Laboratory Manual (3^{1d} Ed.), Vols. 1-3, Cold Spring Harbor Laboratory, 1989; Current Protocols In Molecular Biology, Ausubel, ed. John Wiley & Sons, Inc., New York, 2001; Laboratory Techniques In Biochemistry And Molecular Biology: Hybridization With Nucleic Acid Probes, Part 1. Theory and Nucleic Acid Preparation, Tijssen, ed. Elsevier, N.Y., 1993.

### Specific Ligand Binding

As discussed herein, alteration in a chromosome region occupied by a FGF13 gene or alteration in expression of a FGF13 gene, can also be detected by screening for alteration(s) in a sequence or expression level of a polypeptide encoded by a FGF13 gene. Different types of ligands can be used, such as specific antibodies. In one embodiment, the sample is contacted with an antibody specific for a polypeptide encoded by a FGF13 gene and the formation of an immune complex is subsequently determined. Various methods for detecting an immune complex can be used, such as ELISA, radioimmunoassays (RIA) and immuno-enzymatic assays (IEMA). In one embodiment, levels are measured by ELISA using an antibody directed to SEQ ID NO: 1, 3, 5, 7, 9, or 11; western blot using an antibody directed to SEQ ID NO: 1, 3, 5, 7, 9, or 11; mass spectroscopy, isoelectric focusing, or electrophoresis-based techniques targeting epitopes of SEQ ID NO: 1, 3, 5, 7, 9, or 11; or a combination thereof.

For example, an antibody can be a polyclonal antibody, a monoclonal antibody, as well as fragments or derivatives thereof having substantially the same antigen specificity. Fragments include Fab, Fab′2, or CDR regions. Derivatives include single-chain antibodies, humanized antibodies, or poly-functional antibodies. An antibody specific for a polypeptide encoded by a FGF13 gene can be an antibody that selectively binds such a polypeptide, namely, an antibody raised against a polypeptide encoded by a FGF13 gene or an epitope-containing fragment thereof. Although non-specific binding towards other antigens can occur, binding to the target polypeptide occurs with a higher affinity and can be reliably discriminated from non-specific binding. In one embodiment, the method can comprise contacting a sample from the subject with an antibody specific for a wild type or an altered form of a polypeptide encoded by a FGF13 gene, and determining the presence of an immune complex. Optionally, the sample can be contacted to a support coated with antibody specific for the wild type or altered form of a polypeptide encoded by a FGF13 gene. In one embodiment, the sample can be contacted simultaneously, or in parallel, or sequentially, with various antibodies specific for different forms of a polypeptide encoded by a FGF13 gene, such as a wild type and various altered forms thereof.

### Gene Therapy and Protein Replacement Methods

Delivery of nucleic acids into viable cells can be effected ex vivo, in situ, or in vivo by use of vectors, such as viral vectors (e.g., lentivirus, adenovirus, adeno-associated virus, or a retrovirus), or ex vivo by use of physical DNA transfer methods (e.g., liposomes or chemical treatments). Non-limiting techniques suitable for the transfer of nucleic acid into mammalian cells in vitro include the use of liposomes, electroporation, microinjection, cell fusion, DEAE-dextran, and the calcium phosphate precipitation method (See, for example, Anderson, (1998) Nature, 392(6679):25 (1998)). Introduction of a nucleic acid or a gene encoding a polypeptide of the invention can also be accomplished with extrachromosomal substrates (transient expression) or artificial chromosomes (stable expression). Cells can also be cultured ex vivo in the presence of therapeutic compositions of the present invention in order to proliferate or to produce a desired effect on or activity in such cells. Treated cells can then be introduced in vivo for therapeutic purposes.

Nucleic acids can be inserted into vectors and used as gene therapy vectors. A number of viruses have been used as gene transfer vectors, including papovaviruses, e.g., SV40 (Madzak et al., (1992) J Gen Virol. 73(Pt 6):1533-6), adenovirus (Berkner (1992) Curr Top Microbial Immunol. 158:39-66; Berkner (1988) Biotechniques, 6(7):616-29; Gorziglia and Kapikian (1992) J Virol. 66(7):4407-12; Quantin et al., (1992) Proc Natl Acad Sci USA. 89(7):2581-4; Rosenfeld et al., (1992) Cell. 68(1):143-55; Wilkinson et al., (1992) Nucleic Acids Res. 20(9):2233-9; Stratford-Perricaudet et al., (1990) Hum Gene Ther. 1(3):241-56), vaccinia virus (Moss (1992) Curr Opin Biotechnol. 3(5):518-22), adeno-associated virus (Muzyczka, (1992) Curr Top Microbial Immunol. 158:97-129; Ohi et al., (1990) Gene. 89(2):279-82), herpesviruses including HSV and EBV (Margolskee (1992) Curr Top Microbial Immunol. 158:67-95; Johnson et al., (1992) Brain Res Mol Brain Res. 12(1-3):95-102; Fink et al., (1992) Hum Gene Ther. 3(1):11-9; Breakefield and Geller (1987) Mol Neurobiol. 1(4):339-71; Freese et al., (1990) Biochem Pharmacol. 40(10):2189-99), and retroviruses of avian (Bandyopadhyay and Temin (1984) Mol Cell Biol. 4(4):749-54; Petropoulos et al., (1992) J Virol. 66(6):3391-7), murine (Miller et al. (1992) Mol Cell Biol. 12(7):3262-72; Miller et al., (1985) J Virol. 55(3):521-6; Sorge et al., (1984) Mol Cell Biol. 4(9):1730-7; Mann and Baltimore (1985) J Virol. 54(2):401-7; Miller et al., (1988) J Virol. 62(11):4337-45), and human origin (Shimada et al., (1991) J Clin Invest. 88(3):1043-7; Helseth et al., (1990) J Viral. 64(12):6314-8; Page et al., (1990) J Virol. 64(11):5270-6; Buchschacher and Panganiban (1992) J Virol. 66(5):2731-9).

Non-limiting examples of in vivo gene transfer techniques include transfection with viral (e.g., retroviral) vectors (see U.S. Pat. No. 5,252,479, which is incorporated by reference in its entirety) and viral coat protein-liposome mediated transfection (Dzau et al., (1993) Trends in Biotechnology 11:205-210), incorporated entirely by reference). For example, naked DNA vaccines are generally known in the art; see Brower, (1998) Nature Biotechnology, 16:1304-1305, which is incorporated by reference in its entirety. Gene therapy vectors can be delivered to a subject by, for example, intravenous injection, local administration (see, e.g., U.S. Pat. No. 5,328,470) or by stereotactic injection (see, e.g., Chen, et al., (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

For reviews of gene therapy protocols and methods see Anderson et al. (1992) Science 256:808-813; U.S. Pat. Nos. 5,252,479, 5,747,469, 6,017,524, 6,143,290, 6,410,010 6,511,847, 8,398,968; and 8,404,653; and U.S. Application Publication Nos. 2002/0077313 and 2002/00069, which are all hereby incorporated by reference in their entireties. For an example of gene therapy treatment in humans see Porter et al., NEJM 2011 365:725-733 and Kalos et al. Sci. Transl. Med. 2011. 201 3(95):95. For additional reviews of gene therapy technology, see Friedmann (1989) Science, 244:1275-1281; Verma, Scientific American: 68-84 (1990); Miller (1992) Nature, 357: 455-460; Kikuchi et al. (2008) J Dermatol Sci. 50(2):87-98; Isaka et al. (2007) Expert Opin Drug Deliv. 4(5):561-71; Jager et al. (2007) Curr Gene Ther. 7(4):272-83; Waehler et al. (2007) Nat Rev Genet. 8(8):573-87; Jensen et al. (2007) Ann Med. 39(2):108-15; Henveijer et al. (2007) Gene Ther. 14(2):99-107; Eliyahu et al. (2005) Molecules 10(1):34-64; and Altaras et al. (2005) Adv Biochem Eng Biotechnol. 99:193-260, all of which are hereby incorporated by reference in their entireties.

These methods described herein are by no means all-inclusive, and further methods to suit the specific application is understood by the ordinary skilled artisan. Moreover, the effective amount of the compositions can be further approximated through analogy to compounds known to exert the desired effect.

### Protein Delivery Methods

Protein replacement therapy can increase the amount of protein by exogenously introducing wild-type or biologically functional protein by way of infusion. A replacement polypeptide can be synthesized according to known chemical techniques or can be produced and purified via known molecular biological techniques. Protein replacement therapy has been developed for various disorders. For example, a wild-type protein can be purified from a recombinant cellular expression system (e.g., mammalian cells or insect cells-see U.S. Pat. No. 5,580,757 to Desnick et al.; U.S. Pat. Nos. 6,395,884 and 6,458,574 to Selden et al.; U.S. Pat. No. 6,461,609 to Calhoun et al.; U.S. Pat. No. 6,210,666 to Miyamura et al.; U.S. Pat. No. 6,083,725 to Selden et al.; U.S. Pat. No. 6,451,600 to Rasmussen et al.; U.S. Pat. No. 5,236,838 to Rasmussen et al. and U.S. Pat. No. 5,879,680 to Ginns et al.), human placenta, or animal milk (see U.S. Pat. No. 6,188,045 to Reuser et al.), or other sources known in the art. After the infusion, the exogenous protein can be taken up by tissues through non-specific or receptor-mediated mechanism.

A polypeptide encoded by a FGF13 gene can also be delivered in a controlled release system. For example, the polypeptide can be administered using intravenous infusion, an implantable osmotic pump, a transdermal patch, liposomes, or other modes of administration. In one embodiment, a pump can be used (see Sefton (1987) Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, (1983) J. Macromol. Sci, Rev. Macroinol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target thus requiring only a fraction of the systemic dose (see, e.g., Goodson, in Medical Applications of Controlled Release, supra, vol. 2, pp. 115-138 (1984)). Other controlled release systems are discussed in the review by Langer (Science (1990) 249:1527-1533)).

### Pharmaceutical Compositions and Administration for Therapy

FGF13 proteins and FGF13 modulating compounds of the invention can be administered to the subject once (e.g., as a single injection or deposition). Alternatively, FGF13 proteins and FGF13 modulating compounds can be administered once or twice daily to a subject in need thereof for a period of from about two to about twenty-eight days, or from about seven to about ten days. FGF13 proteins and FGF13 modulating compounds can also be administered once or twice daily to a subject for a period of 1, 2, 3, 4, 5, 6, 7, 8, 9, 10, 11, 12 times per year, or a combination thereof. Furthermore, FGF13 proteins and FGF13 modulating compounds of the invention can be co-administrated with another therapeutic. Where a dosage regimen comprises multiple administrations, the effective amount of the FGF13 proteins and FGF13 modulating compounds administered to the subject can comprise the total amount of gene product administered over the entire dosage regimen.

FGF13 proteins and FGF13 modulating compounds can be administered to a subject by any means suitable for delivering the FGF13 proteins and FGF13 modulating compounds to cells of the subject, such as the dermis, epidermis, dermal papilla cells, or hair follicle cells. For example, FGF13 proteins and FGF13 modulating compounds can be administered by methods suitable to transfeet cells. Transfection methods for eukaryotic cells are well known in the art, and include direct injection of the nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

The compositions of this invention can be formulated and administered to reduce the symptoms associated with a hair-loss disorder by any means that produces contact of the active ingredient with the agent's site of action in the body of a subject, such as a human or animal (e.g., a dog, cat, or horse). They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

A therapeutically effective dose of FGF13 modulating compounds can depend upon a number of factors known to those or ordinary skill in the art. The dose(s) of the FGF13 modulating compounds can vary, for example, depending upon the identity, size, and condition of the subject or sample being treated, further depending upon the route by which the composition is to be administered, if applicable, and the effect which the practitioner desires the FGF13 modulating compounds to have upon the nucleic acid or polypeptide of the invention. These amounts can be readily determined by a skilled artisan. Any of the therapeutic applications described herein can be applied to any subject in need of such therapy, including, for example, a mammal such as a dog, a cat, a cow, a horse, a rabbit, a monkey, a pig, a sheep, a goat, or a human.

Pharmaceutical compositions for use in accordance with the invention can be formulated in conventional manner using one or more physiologically acceptable carriers or excipients. The therapeutic compositions of the invention can be formulated for a variety of routes of administration, including systemic and topical or localized administration. Techniques and formulations generally can be found in Remington's The Science and Practice of Pharmacy, 20^{th} ed. Lippincott Williams & Wilkins., Philadelphia, Pa. (2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers such as Hank's solution or Ringer's solution. In addition, the therapeutic compositions can be formulated in solid form and redissolved or suspended immediately prior to use. Lyophilized forms are also included. Pharmaceutical compositions of the present invention are characterized as being at least sterile and pyrogen-free. These pharmaceutical formulations include formulations for human and veterinary use.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

The invention also provides for a kit that comprises a pharmaceutically acceptable carrier and a FGF13 modulating compound (for example one described herein or one identified using the screening assays of the invention) packaged with instructions for use. For modulators that are antagonists of the activity of a FGF13 protein, or which reduce the expression of a FGF13 protein, the instructions would specify use of the pharmaceutical composition for promoting the loss of hair on the body surface of a mammal (for example, arms, legs, bikini area, face).

For FGF13 modulating compounds that are agonists of the activity of a FGF13 protein or increase the expression of one or more proteins encoded by the FGF13 gene, the instructions would specify use of the pharmaceutical composition for regulating hair growth. In one embodiment, the instructions would specify use of the pharmaceutical composition for the treatment of hair loss disorders.

A pharmaceutical composition containing a FGF13 modulating compound can be administered in conjunction with a pharmaceutically acceptable carrier, for any of the therapeutic effects discussed herein. Such pharmaceutical compositions can comprise, for example antibodies directed to polypeptides encoded by genes comprising a FGF13 gene, or variants thereof, or agonists and antagonists of a polypeptide encoded by a FGF13 gene. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

Sterile injectable solutions can be prepared by incorporating the FGF13 modulating compound (e.g., a polypeptide or antibody) in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

In some embodiments, the FGF13 modulating compound can be applied via transdermal delivery systems, which slowly releases the active compound for percutaneous absorption. Permeation enhancers can be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Various routes of administration and various sites of cell implantation can be utilized, such as, subcutaneous or intramuscular, in order to introduce the aggregated population of cells into a site of preference. Once implanted in a subject (such as a mouse, rat, or human), the aggregated cells can then stimulate the formation of a hair follicle and the subsequent growth of a hair structure at the site of introduction. In another embodiment, transfected cells (for example, cells expressing a protein encoded by a FGF13 gene are implanted in a subject to promote the formation of hair follicles within the subject. In further embodiments, the transfected cells are cells derived from the end bulb of a hair follicle (such as dermal papilla cells or dermal sheath cells). Aggregated cells (for example, cells grown in a hanging drop culture) or transfected cells (for example, cells produced as described herein) maintained for 1 or more passages can be introduced (or implanted) into a subject (such as a rat, mouse, dog, cat, human, and the like).

“Subcutaneous” administration can refer to administration just beneath the skin (i.e., beneath the dermis). Generally, the subcutaneous tissue is a layer of fat and connective tissue that houses larger blood vessels and nerves. The size of this layer varies throughout the body and from person to person. The interface between the subcutaneous and muscle layers can be encompassed by subcutaneous administration.

This mode of administration can be feasible where the subcutaneous layer is sufficiently thin so that the factors present in the compositions can migrate or diffuse from the locus of administration and contact the hair follicle cells responsible for hair formation. Thus, where intradermal administration is utilized, the bolus of composition administered is localized proximate to the subcutaneous layer.

Administration of the cell aggregates (such as DP or DS aggregates) is not restricted to a single route, but can encompass administration by multiple routes. For instance, exemplary administrations by multiple routes include, among others, a combination of intradermal and intramuscular administration, or intradermal and subcutaneous administration. Multiple administrations can be sequential or concurrent. Other modes of application by multiple routes will be apparent to the skilled artisan.

In other embodiments, this implantation method will be a one-time treatment for some subjects. In further embodiments of the invention, multiple cell therapy implantations will be required. In some embodiments, the cells used for implantation will generally be subject-specific genetically engineered cells. In another embodiment, cells obtained from a different species or another individual of the same species can be used. Thus, using such cells can require administering an immunosuppressant to prevent rejection of the implanted cells. Such methods have also been described in U.S. Pat. No. 7,419,661 and PCT application publication WO 2001/32840, and are hereby incorporated by reference.

### Inhibitors

The inhibitors can comprise peptides (such as antibodies or fragments thereof), small molecules, nucleic acids (such as siRNA or antisense RNA), or other agents) that can bind to a polypeptide molecule encoded by a gene of interest and/or molecules that have an inhibitory effect on the biological activity of a protein of interest or its expression.

As used herein, a “FGF13 inhibitor” refers to a compound that interacts with a FGF13 gene or a FGF13 protein or polypeptide and inhibits its activity and/or its expression. The compound can decrease the activity or expression of a protein encoded by FGF13.

In one embodiment, a FGF13 inhibitor can be a peptide fragment that binds a protein comprising SEQ ID NO: 1, 3, 5, 7, 9, or 11. For example, the fragment can encompass any portion of about 8 consecutive amino acids of SEQ ID NO: 1, 3, 5, 7, 9, or 11. The fragment can comprise about 10 consecutive amino acids, about 20 consecutive amino acids, about 30 consecutive amino acids, about 40 consecutive amino acids, about 50 consecutive amino acids, about 60 consecutive amino acids, or about 75 consecutive amino acids of SEQ ID NO: 1, 3, 5, 7, 9, or 11. Fragments include all possible amino acid lengths between and including about 8 and about 100 amino acids, for example, lengths between about 10 and about 100 amino acids, between about 15 and about 100 amino acids, between about 20 and about 100 amino acids, between about 35 and about 100 amino acids, between about 40 and about 100 amino acids, between about 50 and about 100 amino acids, between about 70 and about 100 amino acids, between about 75 and about 100 amino acids, or between about 80 and about 100 amino acids. These peptide fragments can be obtained commercially or synthesized via liquid phase or solid phase synthesis methods (Atherton et al., (1989) Solid Phase Peptide Synthesis: a Practical Approach. IRL Press, Oxford, England).

An inhibitor of the invention can be a protein, such as an antibody (monoclonal, polyclonal, humanized, chimeric, or fully human), or a binding fragment thereof, directed against a polypeptide encoded by SEQ ID NO: 1, 3, 5, 7, 9, or 11. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fabr)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

An inhibitor of the invention can also be a small molecule that binds to a protein and disrupts its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate a protein can be identified via in silky screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries (Werner et al., (2006) Brief Funct. Genomic Proteomic 5(1):32-6). In some embodiments, the agent is a small molecule that binds, interacts, or associates with a target protein or RNA. Such a small molecule can be an organic molecule that, when the target is an intracellular target, is capable of penetrating the lipid bilayer of a cell to interact with the target. Small molecules include, but are not limited to, toxins, chelating agents, metals, and metalloid compounds. Small molecules can be attached or conjugated to a targeting agent so as to specifically guide the small molecule to a particular cell.

An inhibitor or agonist of the invention can be incorporated into pharmaceutical compositions suitable for administration, for example the inhibitor and a pharmaceutically acceptable carrier.

According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Any of the therapeutic applications described herein can be applied to any subject in need of such therapy, including, for example, a mammal such as a dog, a cat, a cow, a horse, a rabbit, a monkey, a pig, a sheep, a goat, or a human.

A pharmaceutical composition of the invention can be administered in conjunction with a pharmaceutically acceptable carrier, for any of the therapeutic effects discussed herein. Such pharmaceutical compositions can comprise, for example antibodies directed to polypeptides. The compositions can be administered alone or in combination with at least one other agent, such as a stabilizing compound, which can be administered in any sterile, biocompatible pharmaceutical carrier including, but not limited to, saline, buffered saline, dextrose, and water. The compositions can be administered to a patient alone, or in combination with other agents, drugs or hormones.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the inhibitor (e.g., a polypeptide or antibody or small molecule) or agonist of the invention in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier and subsequently swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

Those skilled in the art will recognize, or be able to ascertain, using no more than routine experimentation, numerous equivalents to the specific substances and procedures described herein. Such equivalents are considered to be within the scope of this invention, and are covered by the following claims.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Genetic Basis of Hypertrichosis

Hypertrichosis is defined as excessive hair growth for a particular site of the body or age of a patient that is not hormone-dependent. Hypertrichoses are characterized on the basis of multiple criteria: cause (genetic or acquired), age of onset, extent of hair distribution (universal or localized) and affected sites. Several different forms of hypertrichosis are being studied in humans, including X-linked hypertrichosis (OMIM 307150), generalized hypertrichosis terminalis with or without gingival hyperplasia (CGHT; OMIM 135400), autosomal recessive hypertrichosis, Cantu syndrome (OMIM 239850), Ambras type hypertrichosis (AS; OMIM 145701) and autosomal recessive trichomegaly (OMIM 190330).

Whereas many additional anomalies are associated with hypertrichosis, studies have focused on a subset of disorders with congenital hypertrichosis which present with excessive hair as the primary clinical feature. A consanguineous family from Mexico with X-linked hypertrichosis, and three consanguineous families from Pakistan, one with autosomal recessive hypertrichosis and two with autosomal recessive trichomegaly, have been identified. The genetic basis of hypertrichosis remains undefined.

The following projects will be undertaken:
Perform linkage analysis on hypertrichosis families and search for CNVs (copy number variations) using the SNP data from linkage
Perform whole genome or whole exome sequencing to identify mutations
Analyze the function of candidate genes

To date, the genes that control increased density of hair follicles or increased caliber of the hair shaft remain unknown. Without being bound by theory, and based on the patterns of inheritance within the families identified, the syndromes are inherited. The genetic basis of this rare class of disorders will be defined using modern high-throughput genomic techniques, and the function of the candidate genes identified will be defined.

Background of Hypertrichosis

Hypertrichosis syndromes fall under the larger umbrella of ectodermal dysplasias, which are characterized by abnormal development of the hair, skin, nails, teeth and/or eccrine glands. While these appendages vary greatly in their shape and function, they share several common developmental features, namely, formation through a series of interactions between the epithelia and adjacent mesenchyme during embryogenesis. Interestingly, many additional anomalies are associated with hypertrichosis. Members of the X-linked hypertrichosis family that was identified also exhibit dental anomalies and deafness. Moreover, generalized hypertrichosis terminalis is often associated with gingival hyperplasia, Cantu syndrome is additionally characterized by skeletal dysplasia and cardiomegaly, and Ambras syndrome patients commonly present with facial dysmorphologies and bone abnormalities. Despite the wide range phenotypes of these syndromes, the causative mechanism(s) underlying human hypertrichoses have remained elusive. A better understanding of ectodermal structure formation can provide critical insight on a wide range of birth defects and the myriad forms of ectodermal dysplasias, as well as these related developmental processes. In particular, without being bound by theory, new genes and variants that regulate the formation of hair follicles and additional tissues throughout the body during development will be identified.

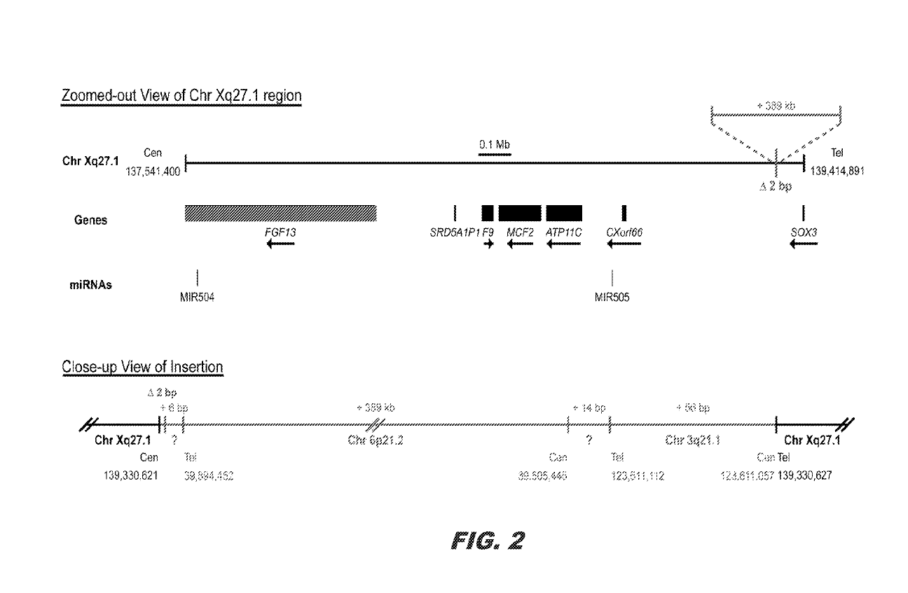

A 389 kb interchromosomal insertion at chromosome Xq27.1 has recently been identified in a family from Mexico with X-linked hypertrichosis (CGH), using WGS (see above). An interchromosomal insertion or interchromosomal insertional translocation is a genomic rearrangement where part of one chromosome is intercalated into another, non-homologous chromosome. The insertion that was identified contains a 389 kb segment of chromosome 6p21.2 in the reverse orientation, as well as a 56 bp segment of chromosome 3q21.1 in the reverse orientation, separated by 14 bp of unknown origin. In addition, the insertion also contains a 6 bp sequence of unknown origin at the centromeric breakpoint, and results in a 2 bp deletion at this junction (FIG. 2). Importantly, the insertion completely cosegregates with the disease in this family.

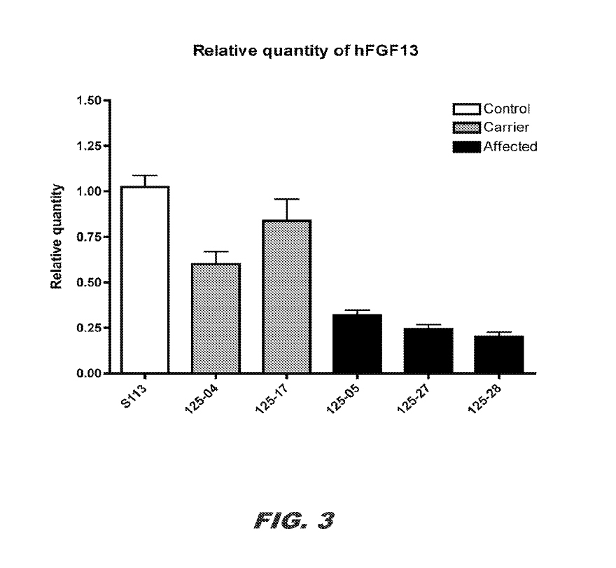

The duplication is located near a few genes, of which FGF13 is of interest because of the known expression of FGF13 in the bulge stem cell compartment of the hair follicle (Kawano et al., 2004). FGF13 levels are significantly decreased in the Mexican CGH patients (FIG. 3). The relative quantity of FGF13 was assessed in CGH patients using the same methods described above which showed that these patients have an approximately 4-fold decrease in the amount of FGF13 when compared to control samples (p<0.001).

Expression of X Chromosome Genes Surrounding Insertion Site

To further characterize the effect of the chromosomal translocation on the genes surrounding the insertion site in the X chromosome, the relative quantity of F9, MCF2, ATP11C, CXorf66, and SOX3 will be assessed according to the procedures described above. These genes reside both downstream of the insertion (SOX3) and between the insertion and FGF13 (FIG. 2). Whether these genes are expressed in normal scalp tissue will be examined using standard PCR techniques. The genes expressed in scalp will then be quantified in the CGH patients relative to control scalp tissue as performed for FGF13.

Immunofluorescence studies will next be conducted using antibodies specific to those genes expressed in the hair follicle to ascertain if there are any changes in protein levels. Following common laboratory procedures, tissues samples from the affected, carrier and control individuals will be embedded in O.C.T. TissueTek (Sakura Torrence, Calif., USA) and a microtome cryostat will be used to create individual slides containing hair follicle sections for analysis. The sections will be triple stained with K14, an outer root sheath marker; CD200, a bulge marker; and the selected antibody. Antibody staining will be visualized using appropriate secondary fluorophores. The same immunofluorescent studies will be performed on FGF13 expression in order to confirm the changes that are seen in at the transcriptional level using qRT-PCR.

Fluorescence In Situ Hybridization

To confirm the insertion, fluorescence in situ hybridization (FISH) was performed to confirm and demonstrate the insertion event at a cytogenetic level. A fluorescent probe specific to the portion of chromosome 6 that is translocated to the X chromosome will be used in conjunction with standard karyotyping techniques to visualize the translocation during both the metaphase and interphase portions of mitosis.

Methylation Status of FGF13 Promoter

Bisulfite sequencing will be performed to determine the methylation status of FGF13, helping to assess the effect of the patients 389kb insertion on the activity of this gene. Bisulfite sequencing analysis has been successfully performed on previous occasions (Itoh et al, 2011). Brieflu, 1 μg genomic DNA will be bisulfite converted using the EZ DNA Methylation Gold Kit (Zymo Labs). Resultant bisulfite converted DNA will be amplified using specific primers for FGF13. Primers will be designed using MethPrimer, an online program that detects CpG islands within the promoter and intronic regions of a gene. Amplified products will be ligated into PCRII, transformed, and resultant clones will be sequenced to determine the methylation status within CpG islands.

Functional Studies of FGF13

In order to study the biological functions of FGF13, mouse models can be very useful. For example, an inducible transgenic animal expressing FGF13 in the hair follicle can be used. Taconic (Hudson, N.Y., USA) has developed a mouse model with targeted mutation of FGF13 (model # TF2342) and cryopreserved the line as sperm. This resource will be utilized and a mouse line that lacks FGF13 will be generated in order to better understand the effect of decreased FGF13 on the hair follicle and general development. Both embryonic and postnatal timepoints will be taken and examined for gross developmental defects as well as hair follicle defects. Without being bound by theory, the lack of FGF13 will be most apparent on the amount of hair follicles that develops on the mouse, and previous work has demonstrated significant experience in assessing differences in hair follicle morphogenesis (Fantauzzo et al., 2008b). Altogether the mouse model will provide a promising start for future functional studies to decipher the molecular mechanism(s) behind the CGH phenotype.

Further experiments that are planned include modulating FGF13 levels in normal human keratinoctyes (NHKs) and creating an organotypic skin culture. Modulation of FGF13 levels in NHKs will be achieved via transfection with a plasmid containing either the FGF13 coding sequence of or shRNA specifically tailored to knockdown FGF13. NHKs are readily available and transfections will be performed using Lipfectamine 2000 (Invitrogen) following standard procedures. These NHKs with different levels of FGF13 expression will then be used in the organotypic skin culture system as described in Itoh et al. (2011). This experiment is designed to assess the impact of FGF13 on epidermal proliferation and stratification/differentiation and provide a greater understanding of the role of FGF13 in the hair follicle.

### Example 2

### Linkage Studies and Whole Genome Sequencing (WGS) in Hypertrichosis Families

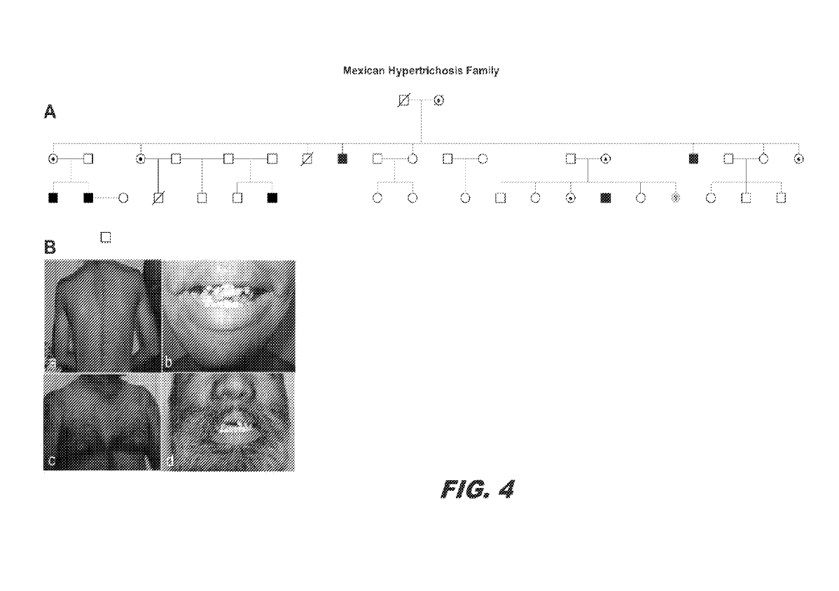

A large kindred from Mexico was ascertained in which congenital universal hypertrichosis with deafness and dental anomalies (FIG. 4) is clearly segregating as an X-linked recessive trait. DNA was collected from 26 members of this family, three of whom are obligate carriers and six of whom are affected. Haplotype analysis and linkage with microsatellite markers defined a 19 Mb region on the X chromosome that co-segregates with the disease.

First, CGH analysis identified a 389 kb duplication on chromosome 6. Next, to identify its location, WGS was performed on one affected male of this Mexican family using the following methods. The DNA was prepared for sequencing according to the Illumina DNA sample preparation kit protocol. In brief, the DNA was randomly fragmented by nebulization followed by end repair, addition of a single A base, adaptor ligation, gel electrophoresis to isolate 300 bp fragments followed by PCR amplification. Next, the size-selected libraries were used for cluster generation on the flow cell. All prepared flow cells were run on the Illumina HiSeq using the paired-end module: the paired-end reads were each 100 bp long.

DNA was aligned to the reference genome (NCBI Build 36 Ensemb1 release 50) using the BWA software (version 0.4.9) (Li and Durbin, 2009). Picard was used to remove potential PCR duplicates via the rmdup command. SAMtools (version 0.1.5c) was used for variant identification, using the pileup command with the −c option and default settings (Li et al., 2009). The variants were then filtered using SAMtool's variation filter with the default settings but removing the filter for a maximum allowed coverage per variant by setting it to 10 million. All variants were screened for quality by only keeping those with a consensus score and quality score of at least 20 (50 for indels) and that had at least 3 reads supporting the variant. Heterozygous indels were also excluded if the ratio of variant reads to reference reads was less than 0.2. The average coverage for this sample was 44.4x. Large structural variants were identified with the Estimation by Read Depth with SNVs (ERDS; http://www.duke.edu/˜mz34/erds.htm) software.

The genomewide identification of functional gene variants is facilitated by SequenceVariantAnalyzer (SVA) (Ge et al., 2011) a suite of bioinformaties tools which allows evaluation and prioritization of variants that may have a functional effect. SVA comprises two modules, the annotation module and the biostatistical module. The annotation module is used to determine and filter the genomic context and potential function of the identified variants. It performs three main functions: (1) determines the novelty of each identified variant; (2) annotates the functions of the identified variants; (3) enables filtering of genetic variants by gene or gene-sets, gene ontology terms, or molecular pathways. The biostatistical module enables the comparison of the newly identified and annotated genetic variants to those already identified, both from public databases and from other sample sets sequenced.

SV-Finder is an unpublished new program for identifying structural variants (SVs) from next generation sequencing data. It utilizes multiple alignment based approaches with emphasis on split-read and pair-end. The main idea is that SVs with supports from more than one approach will be given additional benefit scores; therefore stringent criteria can be used to filter false positive ones.

Importantly, using these methods, a 389 kb chromosomal insertion was identified at chromosome Xq27.1 in the Mexican family with congenital X-linked hypertrichosis (see Example 1).

### Example 3

### Copy Number Variation and Position Effects in Hypertrichosis

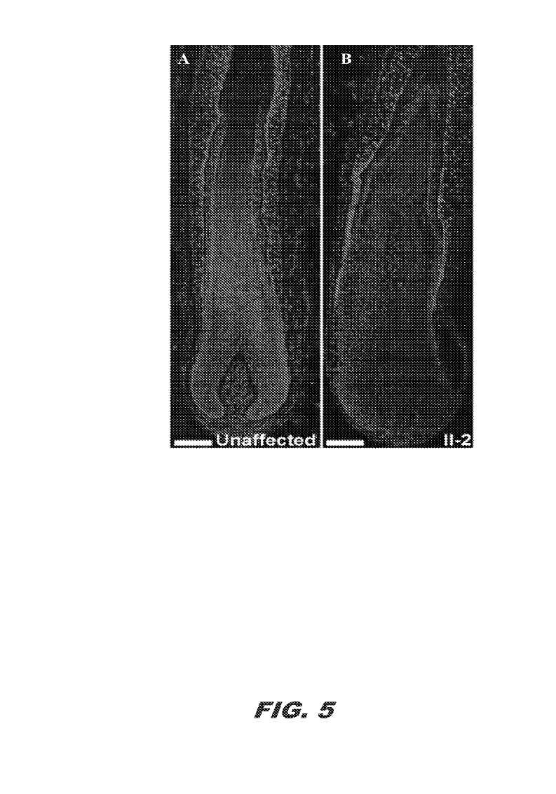

A family was recently identified in which a position effect on the SRY-box transcription factor SOX9 is associated with congenital hypertrichosis terminalis with mild gingival hyperplasia (CGHT) in the affected father and son (proband). The family members were genotyped using the Affymetrix Cytogenetics Whole-Genome 2.7M Array and a series of four new duplications were identified within a 2.4 Mb region in chromosome 17q24.2-q24.3. The telomeric end of this region lies 975 kb upstream of SOX9. Quantitative PCR (qPCR) analysis confirmed that the proband had a 2.24-fold increase (p<0.001) in relative copy number of one amplicon within the region and a 1.54-fold increase (p<0.05) of a second amplicon within the duplication region, as compared to an unaffected control individual. Immunofluorescence analyses was additionally performed on a biopsy taken from the posterior neck of the proband and a sample taken from the scalp of an unaffected control individual, revealing a striking decrease in SOX9 protein expression through the follicle epithelium of the patient (FIG. 5).

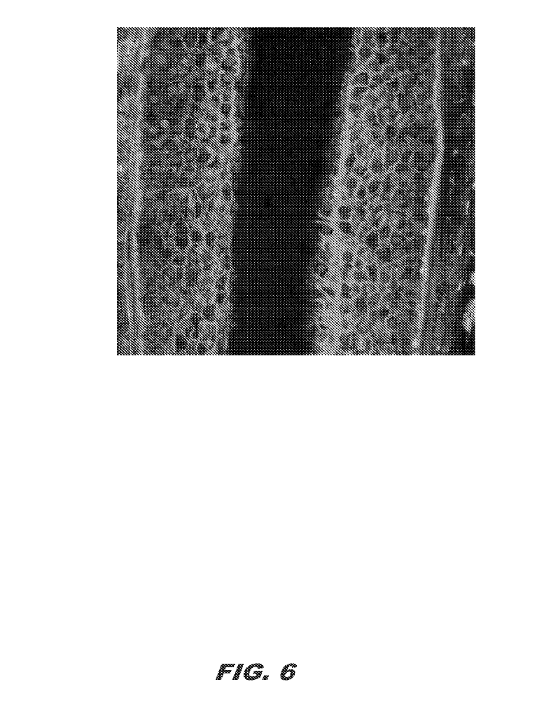

In addition, the DNA of a patient with sporadic Cantu syndrome was analyzed. Using the Affymetrix Cytogenetics Whole-Genome 2.7M array, a 363 kb duplication on chromosome 4q26-q27 was identified. The duplication region encompassed three genes, MYOZ2, USP53 and FABP2. Genomic copy number quantification was performed using qPCR and confirmed the duplications in all three genes. Additionally, the expression of these genes was examined in the hair follicle and USP53 was expressed in the outer root sheath layer (FIG. 6). These data indicate the association of CNVs with the pathogenesis of Cantu syndrome.

A position effect on the zinc-finger transcription factor TRPS1 can be associated with a third form of hypertrichosis in humans, Ambras syndrome (AS) (Fantauzzo et al., 2008). An 11.5 Mb candidate interval was examined for AS on chromosome 8q23-q24 based on cytogenetic breakpoints in three patients. One of these patients had an inversion breakpoint 7.3 Mb downstream of TRPS1. To determine the effect of the inversion on TRPS1 transcript expression, RNA from lymphoblast cell lines derived from the blood of the patient and an unaffected parent was isolated, Quantitative Real-Time PCR analysis of multiple transcripts spanning 8q23-q24 revealed a striking reduction (97.35%, p<0.0001) in TRPS1 expression in the patient (Fantauzzo et al., 2008).

Without being bound by theory, CNVs and position effects are a genetic mechanism of disease in at least four different forms of hypertrichosis.

### Example 4

### Whole Exome Sequence Analysis in Consanguineous Recessive Families

Whole exome sequencing in recessive hypertrichosis families will be performed. To demonstrate familiarity with these methods in other diseases, the example described herein is representative.

A large consanguineous family was ascertained in which peeling skin syndrome noninflammatory type A (OMIM 270300) is segregating as a recessive trait. Whole-genome genotyping was performed with a low density genotyping array on 18 family members and a 15 Mb region on chromosome 19 was identified with strong evidence for allele sharing among affected individuals, with a LOD score of 10.9. Because the region was well-defined, whole-exome sequencing on a single affected family member could be performed. A total of 3,440 variants was identified in the genome of this person, 477 of which were not present in any public databases or a database of ethnically-matched samples. However, only 1 of these variants, located within the gene CHST8, was homozygous and located within the autozygous region. This finding was validated to be a new missense mutation with Sanger sequencing in all members of the pedigree and demonstrated co-segregation of the variant with the trait, and showed the impact of the mutation on protein function using enzymatic assays.

### Example 5

### Functional and Expression Analysis of Newly Identified Disease Genes

The CHST8 gene encodes a member of the carbohydrate sulfotransferase family of proteins, named N-acetylgalactosamine-4-O-sulfotransferase 1 (GaINAc4-ST1), which carries out sulfation of carbohydrates. Immunohistochemistry was performed on normal skin sections with a polyclonal antibody raised against GalNAc4-ST1, and expression of GalNAc4-ST1 was observed throughout the epidermis, predominantly in the granular and cornified layers. In order to investigate the effect of the 229C>T homozygous mutation (which substitutes an arginine amino acid residue by a tryptophan) on the function of GalNAc4-ST1, wild-type and mutant CHST8 cDNA constructs were first generated and expressed in a keratinocyte cell line. Western blot analysis revealed the presence of a lower molecular weight immunoreactive band in addition to the full length protein in cells transfected with the mutant CHST8 construct, which is not observed in cells transfected with the wild type CHST8 construct. Immunofluorescence staining with the polyclonal GalNAc4-ST1 antibody showed that GalNAc4-ST1 co-localizes with the Golgi apparatus, both in cells expressing wild type and mutant recombinant GalNAc4-ST1. A colorimetric assay was also performed for sulfated glycosaminoglycans (GAGs) quantification based on the ability of sulfated GAGs to bind the cationic dye 1,9-dimethylmethylene blue. This method was used to compare the total amount of sulfated GAGs between cells transfected with wild type and mutant CHST8 constructs. Results showed decreased levels of total sulfated GAGs in cells transfected with the mutant CHST8 construct compared to wild type, indicating loss of function of mutant GalNAe4-ST1.

Additionally, the function of a gene that was recently demonstrated as playing a pathogenic role in hereditary leukonychia (porcelain nails or white nails, OMIM 151600) was also similarly analyzed using whole exome sequencing. Four families of Pakistani origin were identified showing features of hereditary leukonychia. Using Affymetrix 10K chips, linkage to chromosome 3p22-p21.3 was established with a LOD score (Z) of 5.1. Pathogenic mutations were identified in the PLCD1 gene, which encodes phosphoinositide-specific phospholipase C delta 1 subunit, a key enzyme in phosphoinositide metabolism, in all four families. PLCD 1 is expressed in the nail matrix and determined by proteomic analysis that it is a component of the human nail plate. Furthermore, mutations in PLCD1 resulted in reduced enzymatic activity of the protein in vitro (see Kiuru et al., 2011).

### Example 6

### Method of Study

To Perform Linkage Analysis on Hypertrichosis Families and to Search for CNVs Using the SNP Data from Linkage

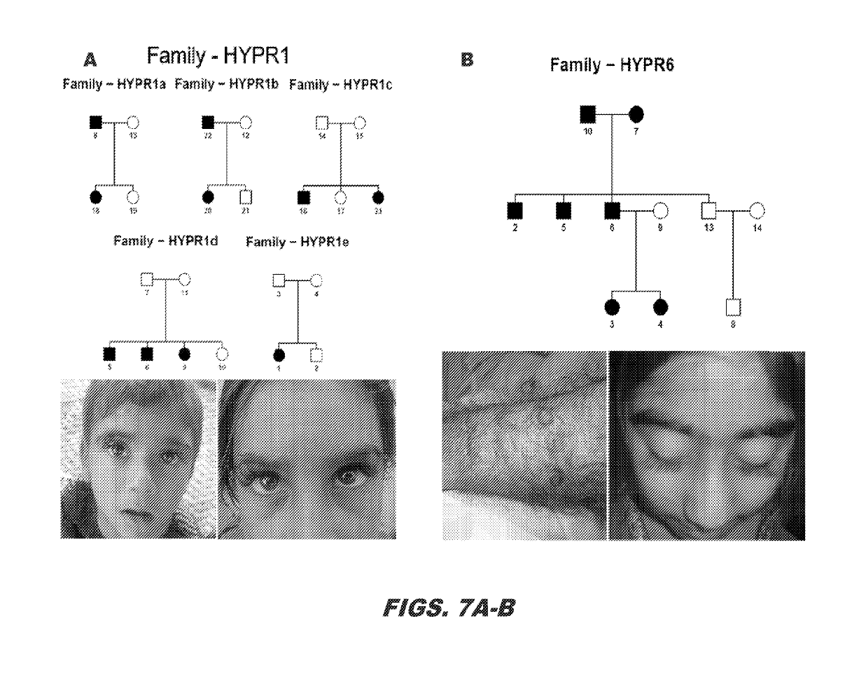

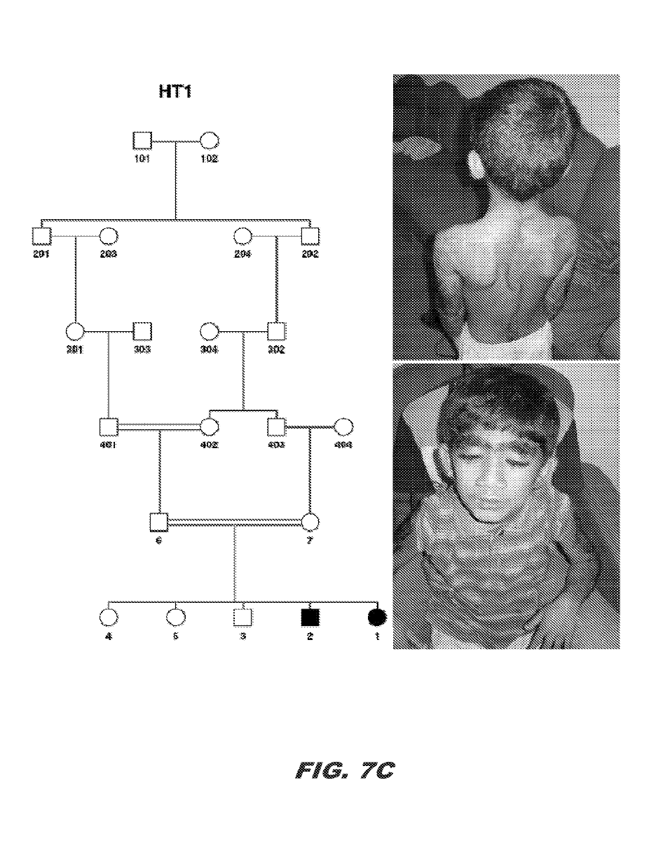

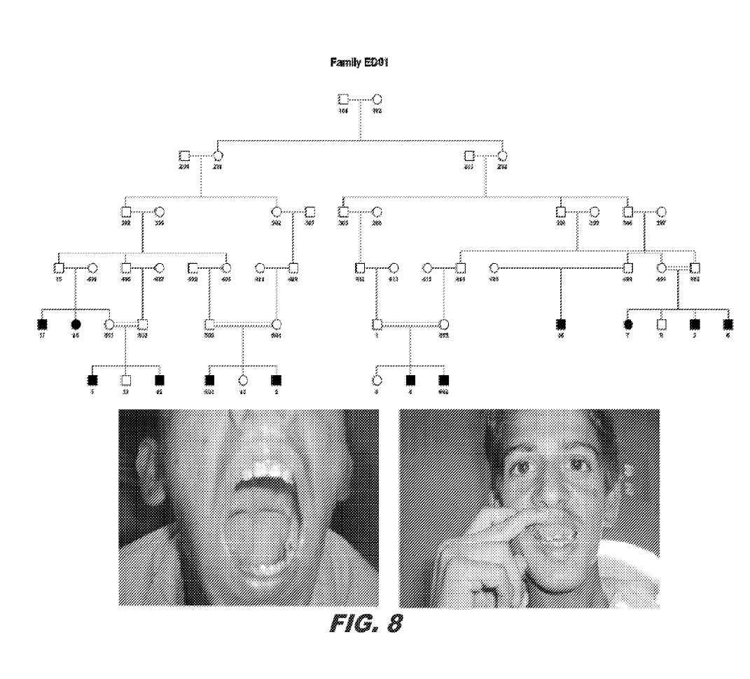

Pedigrees and power analysis for linkage. Gene mapping in consanguineous families has proven to be a powerful method for identifying autosomal recessive disease genes. Four consanguineous families were ascertained, of which two are segregating trichomegaly (OMIM 190330, FIGS. 7A and 7B), one is segregating hypertrichosis (FIG. 7C) and one is segregating gingival hyperplasia (FIG. 8), all of which are consistent with transmission of a recessive disease allele. For one of the families segregating trichomegaly (FIG. 7B), eight members were ascertained, six of whom are affected. For the second family (FIG. 7A), 18 members were ascertained, eight of whom are affected. For the families segregating hypertrichosis (FIG. 7C) and gingival hyperplasia (FIG. 8), 7 members were ascertained, 2 of whom are affected and 22 members, 12 of whom are affected, respectively. Family based linkage studies will be performed on these pedigrees, which have significant power for linkage.

Specifically, as has been done with success with other families, genome-wide scans will first be performed on members of these autosomal recessive families with generalized hypertrichosis, trichomegaly, and gingival hyperplasia using the low density Affymetrix 10K SNP array. Initial analysis will include genome-wide autozygosity mapping to identify regions identical by descent that are shared among affected individuals. Parametric linkage analysis will be performed twice, once using SNP genotypes and once using haplotypes. All tests will assume a recessive mode of inheritance with 100% penetrance and a disease allele frequency of 0.001. Microsatellite markers spanning the regions of autozygosity will be used to genotype members of the family and to detect key recombination events.

Genotyping Using Illumina Human 1M Duo Platform.

The Human 1M Duo beadchip will be used for the genotyping platform because of extensive experience with Illumina arrays. Importantly for this project, this platform contains both SNP markers to be used for mapping and CNV probes, which will be used for analysis of structural variants. Once regions with evidence for harboring a disease allele are identified, fine mapping with microsatellite markers will then be performed to exclude spurious linkage peaks and narrow the intervals. Critical recombination events will define minimal regions of linkage and candidate genes will be prioritized based on functional evidence and/or expression patterns in the literature. These will be sequenced and mutations will be analyzed by comparison to databases and by excluding them as polymorphisms in the general population. These methods have been used successfully to identify numerous new disease genes in Mendelian disorders. (Kiuru et al., 2011; Kurban et al., 2010; Shimomura et al., 2010).

Statistical Analysis for Linkage.

The commercial software package Genespring GT (Agilent) will be used to perform stringent quality control filtering of the data and statistical analysis. Parametric linkage analysis will be performed on subsets of uncorrelated SNP markers and also additionally on haplotypes that will be inferred from the SNP data. Autozygosity mapping will also be conducted on SNPs and haplotypes. By integrating evidence across these statistical methods we will identify a set of regions with evidence for harboring the disease allele. These statistical methods for linkage analysis have been used successfully as evidenced by Kurban et al. (2011) amongst other recent publications. (Kiuru et al., 2011; Kurban et al., 2010; Wajid et al., 2010).

Analysis of Illumina Array Data for CNVs.

Several large-scale chromosomal rearrangements, including translocations, have been associated with hypertrichosis syndromes. (Kim et al., 2010; Sun et al., 2009; Zhu et al., 2011). However, since no gene disruptions have been detected at the inversion breakpoints, these findings point to a more complex role of chromosomal architecture in the pathogenesis of hypertrichosis. In fact, recent evidence points to an association of these syndromes with position effects secondary to copy number variations. One example of this is the identification of a position effect on the transcription factor SOX9 which is associated with CGHT. A position effect is an alteration in gene expression caused by a change in the position of a gene relative to its native chromosomal surroundings. Gene expression can be affected by a variety of mechanisms, for example, disruption of transcriptional regulation in cis and/or modification of the surrounding chromatin structure. The resulting phenotype may be attributed to separation of the gene from a tissue- or temporal-specific modifier of gene expression, such as an enhancer or repressor element. In the case of hypertrichoses, CNVs within the linkage regions may help define the molecular basis of hypertrichosis related syndromes which have unrelated and seemingly disparate clinical features.

The Illumina Human 1M Duo arrays that will be used for the above linkage analysis also contain around 60,000 CNV-targeted markers, covering regions such as segmental duplications, megasatellites, and regions lacking SNPs. As such, we will be able to perform linkage and CNV analysis simultaneously on the same samples. The CNVs will be compared to hundreds of controls in the HapMap data and to several controls performed in our cytogenetics laboratory.

Confirmation and Validation of CNVs Using qPCR.

To confirm any duplications or deletions identified in the CNV analysis, quantitative PCR (qPCR) analysis will be performed using the DNA of the patients of interest as well as that of unaffected control individuals, to examine the relative copy number of amplicons within the vicinity of the anomaly. Reactions will be prepared using Power SYBR Green PCR Master Mix, 500 nM primers and 50 ng of genomic DNA in a 20 μL, reaction volume. qPCR analysis will be performed on an ABI 7300 machine using the following protocol: step 1: 50° C. for 2 min; step 2: 95° C. for 10 min; step 3: 95° C. for 15 s; step 4: 60° C. for 1 min; repeat steps 3 and 4 for 40 cycles. All samples will be run in triplicate for three independent runs and normalized against an internal control, GAPDH. Results will be analyzed with ABI Relative Quantification Study software. Differences in relative copy number between the patient and an unaffected control individual with a p-value less than or equal to 0.05 will be considered significant.

To Perform Whole Genome or Whole Exome Sequencing to Identify Mutations

Whole exome sequencing has recently proven to be an extremely efficient method for identifying genetic lesions that cause Mendelian diseases, primarily because the vast majority of these diseases are caused by mutations in the coding regions of genes. This experiment allows for the simultaneous survey of all coding regions in the genomes of family members to catalogue all variants that are cosegregating with the disease. A battery of bioinformatic approaches can be used to filter through variant lists to identify the causative gene. These methods have been successfully used previously.

Whole Exome or Whole Genome Sequence Analysis.

The SureSelect Human All Exon Kit (Agilent) will be used to capture the exomes of a subset of family members. This kit targets approximately 38 Mb of the genome in a single tube, covering 1.22% of human genomic regions corresponding to the coding exons. Captured exons will then be subject to paired end sequencing on an Applied Biosystems SOLiD 4 next generation sequencing system, and reads will be aligned using the Bioscope software for SOLiD,

Analysis of Data from WGS and Identification of Variants.

Sequence data is first mapped into a reference genome based on the March 2006 human reference sequence (hg18), using the SHRiMP algorithm with its default parameters. SOLiD platform employs a two-base encoding system, where a single variation in the color space solely indicates a sequencing error and two consecutive variations in the color space point to a base change in the nucleotide-space. In the analysis, the reads with a maximum number of two color-space mismatches that are also uniquely mapped to the reference genome will be included. An average 90.1% of the reference genome will be covered in each sample, where the mean depth will be 42 per base. Less restrictive filtering would increase the false-positive rate of candidate genomic variants without improving the coverage to any great extent.

Variant lists are then filtered against public databases of variants (e.g. HapMap, 1000s Genome Project) as well as a population-matched database that we have created from our previous whole exome sequencing experiments. Variants that remain will then be prioritized for validation on the basis of predicted mutational consequences and reported gene functions,

Confirmation and Validation of Sequence Variants.

Sanger sequencing of PCR amplified exons will be used to validate next-generation sequencing results. Validated variants will then be genotyped in all family members to determine patterns of cosegregation with the disease.

Quantification of Gene Expression.

To determine the outcome of putative position effects, chromosomal rearrangements and CNVs on the expression levels of specific transcripts of interest in the hair follicle, quantitative RT-PCR will be performed and gene expression will be compared between affected and non-affected individuals. Genes will be selected on the basis of their relationship with the next genetic region, for example, within, upstream and downstream of a CNV. in order to conduct this experiment, biopsies will be taken from the affected areas of hypertrichosis patients and from matching body site locations of non-affected individuals, RNA will be extracted and gene expression levels will be compared between these biopsies. qPCR analysis will be performed as described, except that cDNA will be used instead of genomic DNA.

### REFERENCES

Fantauzzo, K. A., Bazzi, H., Jahoda, C. A., Christiane, A. M., Dynamic expression of the zinc-finger transcription factor Trps1 during hair follicle morphogenesis and cycling. Gene Expr Patterns. 2008b. 8: 51-7.
Fantauzzo, K. A., Tadin-Strapps, M., You, Y., Mentzer, S. E., Baumeister, F. A., Cianfarani, S., Van Maldergem, L., Warburton, D., Sundberg, J. P., Christiane, A. M., A position effect on TRPS1 is associated with Ambras syndrome in humans and the Koala phenotype in mice. Hum Mol Genet., 2008. 17: 3539-51.
Ge, D., Ruzzo, E. K., Shianna, K. V., He, M., Pelak, K., Heinzen, E. L., Need, A. C., Cirulli, E. T., Maia, J. M., Dickson, S. P., Zhu, M., Singh, A., Allen, A. S., Goldstein, D. B., SVA; software for annotating and visualizing sequenced human genomes. Bioinformatics, 2011.27: p. 1998-2000.
Itoh, M., Kiuru, M., Cairo, M. S., Christiane, A. M., Generation of keratinocytes from no mal and recessive dystrophic epidermolysis bullosa-induced pluripotent stem cells. Proc Nati Acad Sci USA., 2011. 108: 8797-802.
Kim J, Lee G, Choi J R, Kurban M, Christiane A M, Levy B, Kim J H, Ma S H, Lee K A. Ambras syndrome in a Korean patient with balanced pericentric inversion (8)(p 11.2q24.2). J Dermatol Sci, 2010 September; 59(3):204-6.
Kawano M, Suzuki S, Suzuki M, Oki J, Imamura T. Bulge- and basal layer-specific expression of fibroblast growth factor-13 (FHF-2) in mouse skin. J Invest Dermatol. 2004 May; 122(5): 1084-90.
Kiuru M, Kurban M, Itoh M, Petukhova L, Shimomura Y, Wajid M, Christiane A M. Hereditary leukonychia, or porcelain nails, resulting from mutations in PLCD1. Am J Hum Genet. 2011 Jun. 10; 88(6):839-44.
Kurban, M., Wajid, M., Petukhova, L., Shimomura, Y., Christiane, A. M., A nonsense mutation in the HOXD13 gene underlies synpolydactyly with incomplete penetrance. J Hum Genet., 2011. [Epub ahead of print].
Kurban M, Wajid M, Shimomura Y, Christiane A M. A nonsense mutation in the SCN9A gene in congenital insensitivity to pain. Dermatology. 2010; 221(2):179-83.
Li, H. and R. Durbin, Fast and accurate short read alignment with Burrows-Wheeler transform. Bioinformatics, 2009. 25: 1754-1760.
Li, H., Handsaker, B., Wysoker, A., Fennell, T., Ruan, J., Homer, N., Marth, G., Abecasis, G., Durbin, R., 1000 Genome Project Data Processing Subgroup, The Sequence Alignment/Map format and SAMtools. Bioinformatics, 2009. 25: 2078-2079.
Shimomura Y, Wajid M, Petukhova L, Kurban M, Christiane A M. Autosomal-dominant woolly hair resulting from disruption of keratin 74 (KRT74), a potential determinant of human hair texture. Am J Hum Genet. 2010 Apr. 9; 86(4):632-8.
Sun M, Li N, Dong W, Chen Z, Liu Q, Xu Y, He G, Shi Y, Li X, Hao J, Luo Y, Shang D, Ly D, Ma F, Zhang D, Hua R, Lu C, Wen Y, Cao L, Irvine A D, McLean W H, Dong Q, Wang M R, Yu J, He L, Lo W H, Zhang X. Copy-number mutations on chromosome 17q24.2-q24.3 in congenital generalized hypertrichosis terminalis with or without gingival hyperplasia. Am J Hum Genet. 2009 June; 84(6):807-13.
Wajid M, Kurban M, Shimomura Y, Christiane A M. NIPAL4/ichthyin is expressed in the granular layer of human epidermis and mutated in two Pakistani families with autosomal recessive ichthyosis. Dermatology. 2010; 220(1):8-14.
Zhu H, Shang D, Sun M, Choi S, Liu Q, Hao J, Figuera L E, Zhang F, Choy K W, Ao Y, Liu Y, Zhang X L, Yue F, Wang M R, Jin L, Patel P I, Jing T, Zhang X. X-linked congenital hypertrichosis syndrome is associated with interchromosomal insertions mediated by a human-specific palindrome near SOX3. Am J Hum Genet. 2011 Jun. 10; 88(6):819-26
Kawano et al, 2004, Bulge- and Basal Layer-Specific Expression of Fibroblast Growth Factor-13 (FHF-2) in Mouse Skin, Journal of Investigative Dermatology (2004) 122, 1084-1090.
Ohyama et al., 2006, Characterization and isolation of stem cell-enriched human hair follicle bulge cells, J Clin Invest. 2006 116:249-60.
Ohyama et al., 2007, Gene ontology analysis of human hair follicle bulge molecular signature. J Dermatol Sci. 2007 45:147-50.
Zhu et al., 2011, X-linked congenital hypertrichosis syndrome is associated with interchromosomal insertions mediated by a human-specific palindrome near SOX3., Am J Hum Genet. 2011 88:819-26.

### Example 7

### A Position Effect on FGF13 Underlies X-Linked Hypertrichosis

Hypertrichosis describes all forms of excessive hair growth for a given body location and age of an individual that does not depend on androgen stimulation. Inherited hypertrichoses are very rare human disorders, with an estimated incidence of 1 in 1 billion.

Position effects have been defined involving the TRPS1 and SOX9 genes underlying autosomal forms of hypertrichosis, however, the genes that control increased density of hair follicles or caliber of the hair shaft in X-linked hypertrichosis remain unknown.

DNA was analyzied from a Mexican family with X-linked congenital generalized hypertrichosis (CGH) (MIM307150) as well as deafness and dental anomalies. Using whole genome sequencing and comparative genome hybridization analysis, we identified a 389 kb intrachromosomal insertion at an extragenic palindrome site on chromosome Xq27.1 that completely cosegregates with the disease, and confirmed it using FISH. Among the six genes surrounding the insertion, FGF13 levels are significantly decreased in the patients (p<0.001) by approximately 4-fold relative to control samples, whereas mRNA levels of the neighboring genes remained unchanged. FGF13 lies ˜1 Mb away from the 389 kb insertion, therefore, without being bound by theory, a position effect occurs as a result of the chromosomal insertion at Xq27.1, leading to decreased FGF13 expression. FGF13 has been localized to the human hair follicle bulge region using in situ hybridization and the regulatory effects of FGF13 are being determined on bulge stem cells to identify how modulation of this compartment results in the hypertrichosis phenotype.

### Example 8

### A Position Effect on FGF13 is Associated with X-Linked Congenital

Generalized Hypertrichosis

X-linked congenital generalized hypertrichosis (CGH) (OMIM 307150) is an extremely rare condition of hair overgrowth on different body sites. Linkage in a large Mexican family has been previously reported with X-linked CGH co-segregating with deafness, dental and palate anomalies to Xq24-27. Using SNP oligonucleotide microarray analysis (SOMA) and whole-genome sequencing (WGS), a 389 kb interchromosomal insertion was identified at an extragenic palindrome site at Xq27.1 that completely co-segregates with the disease. Among the genes surrounding the insertion, FGF13 mRNA levels were significantly reduced in the affected individuals and immunofluorescence staining revealed a striking decrease in FGF13 localization throughout the outer root sheath of affected hair follicles. Moreover, murine Fgf13 expression was detected in the developing and cycling hair follicle. Without being bound by theory, FGF13 plays a role in hair follicle growth and the hair cycle.

Genetic defects have been identified in two forms of autosomal dominant congenital generalized hypertrichosis associated with copy number variants (CNVs) on chromosome 17q24 and rearrangements on chromosomes 3, 7, and 8 (P5-P7). A position effect on the zinc finger transcription factor TRPS1 is associated with Ambras syndrome congenital hypertrichosis, which was recapitulated in the koala (Koa) mouse hypertrichosis model (P8). Likewise, a position effect on SOX9 is associated with congenital generalized hypertrichosis terminalis (P9). Without being bound by theory, position effects, instances in which a change in gene expression results from altering the location of a gene relative to its native chromosomal position (P10), are mechanisms that can contribute to inherited hypertrichoses (P8).

In this example, the genetic mechanism is discussed that is associated with X-linked congenital generalized hypertrichosis in a family where linkage to chromosome Xq24-27 that was previously reported (P11). A large interchromosomal insertion was identified that leads to decreased expression of a distant gene, FGF13, which is expressed in the hair follicle, providing evidence to support a position effect as the underlying genetic basis of X-linked hypertrichosis.

### Results and Discussion

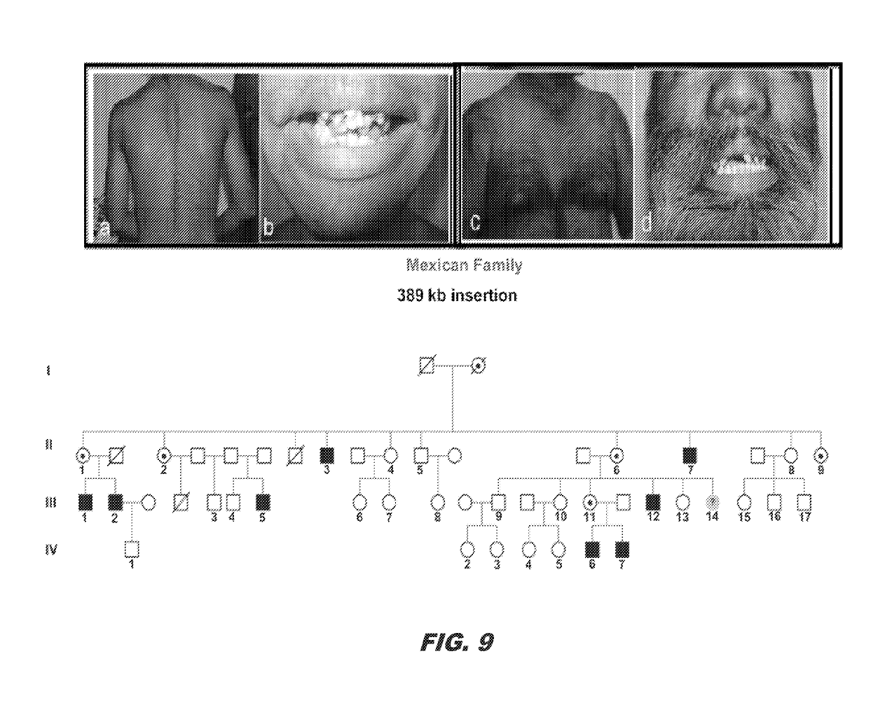

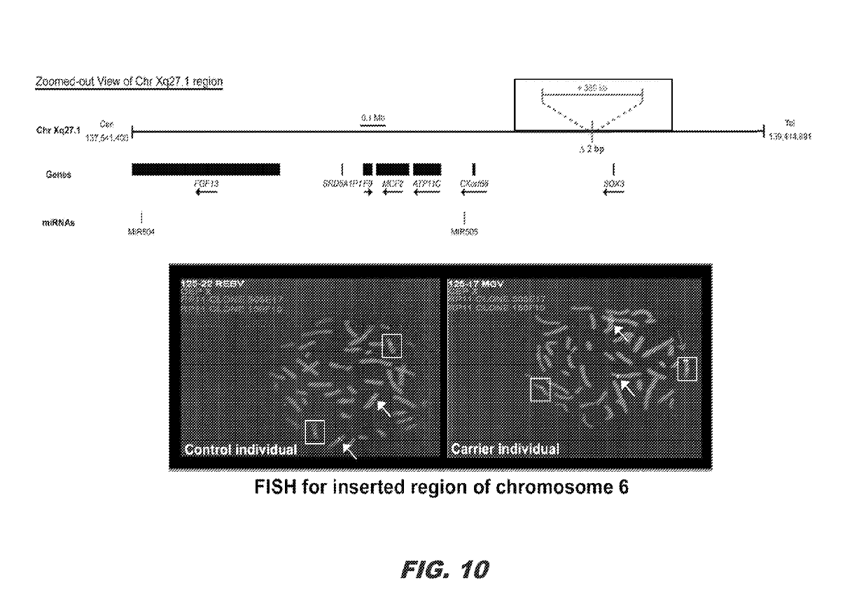

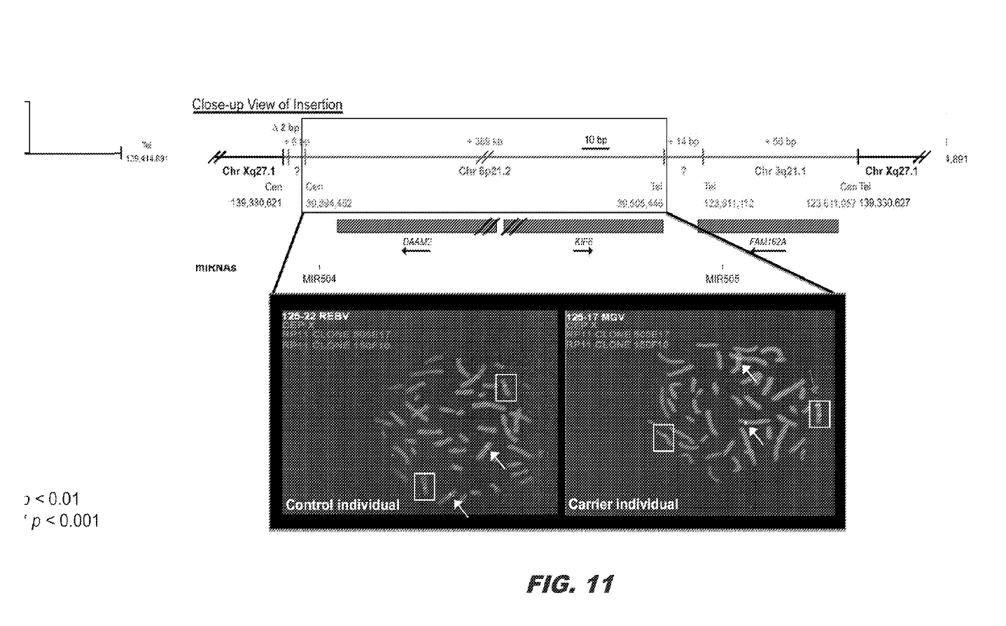

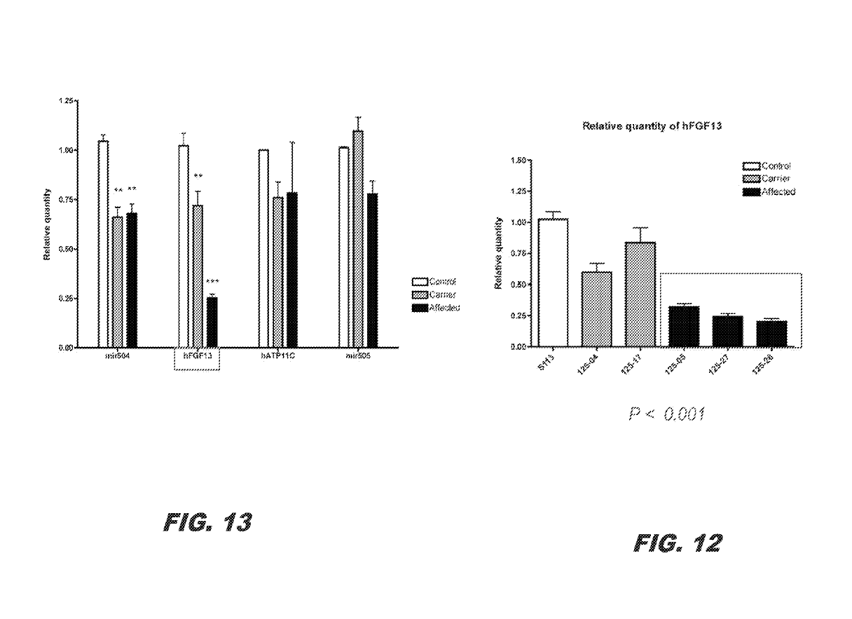

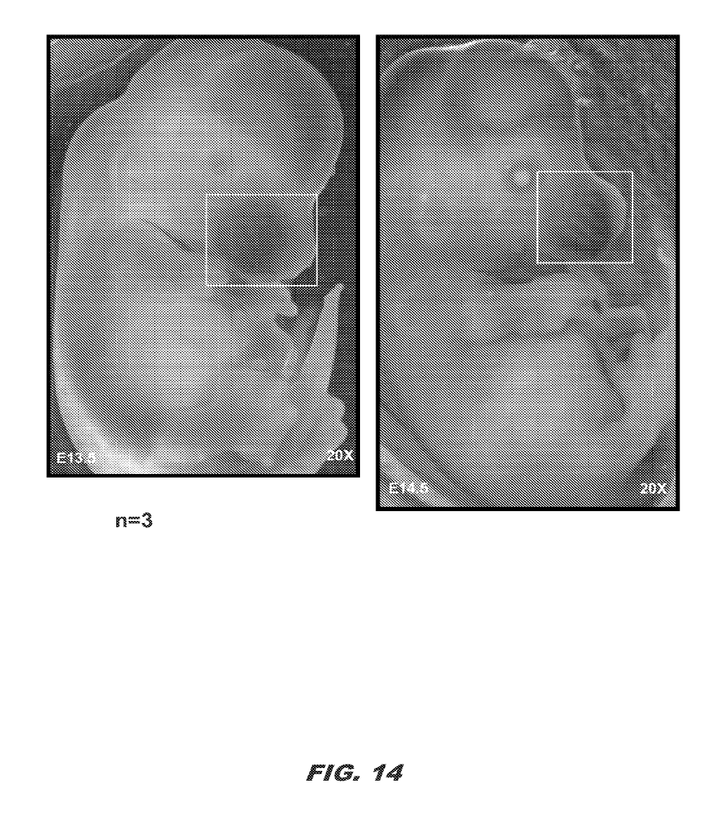

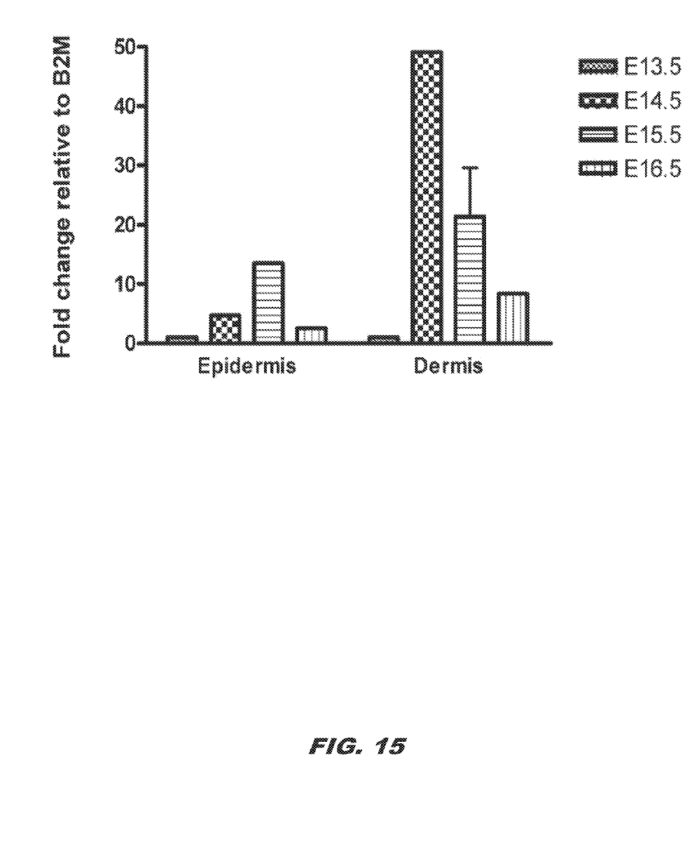

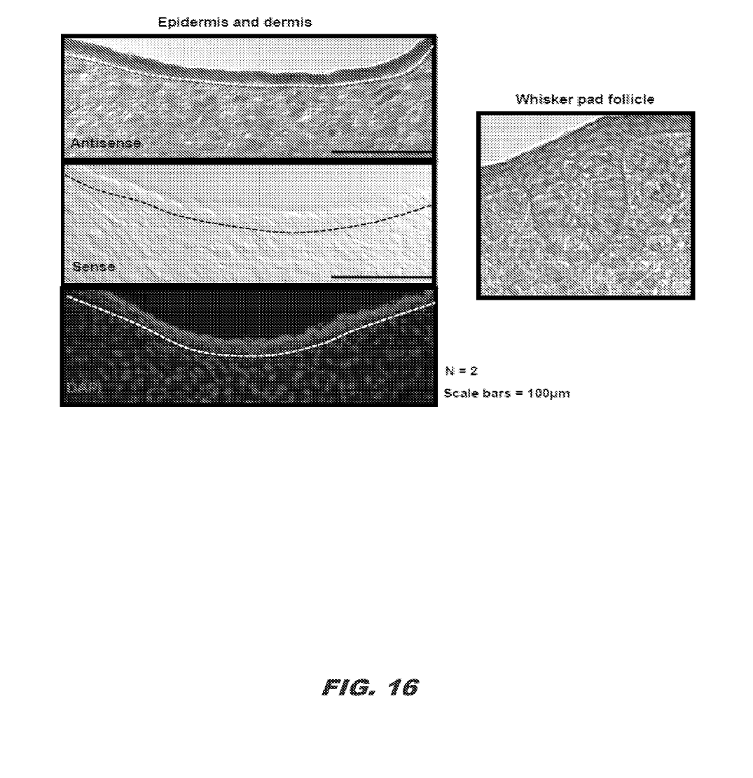

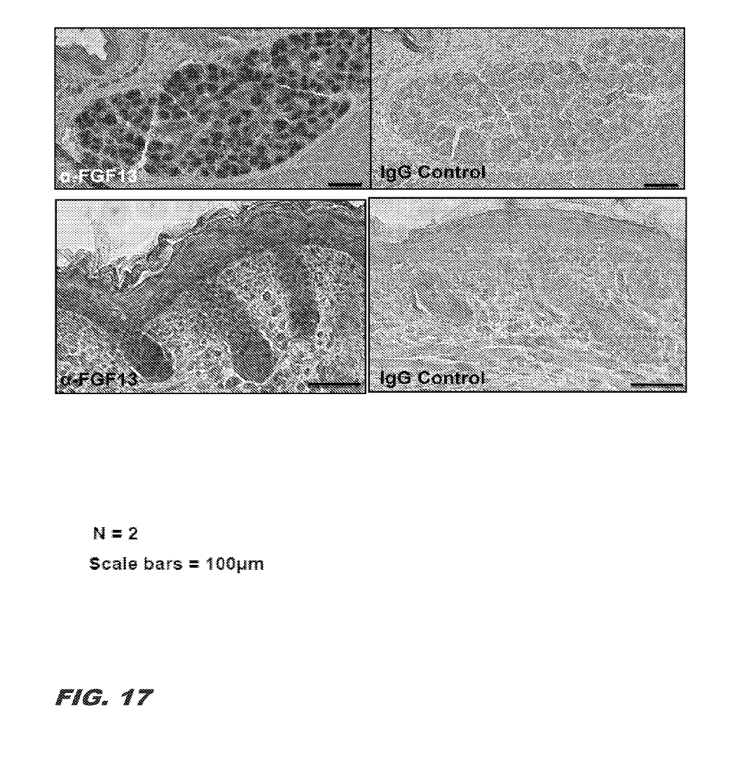

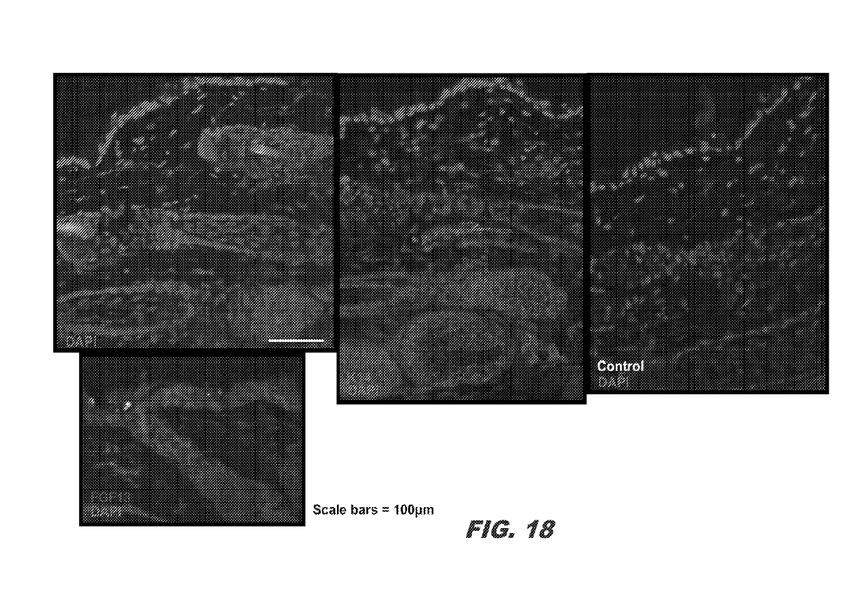

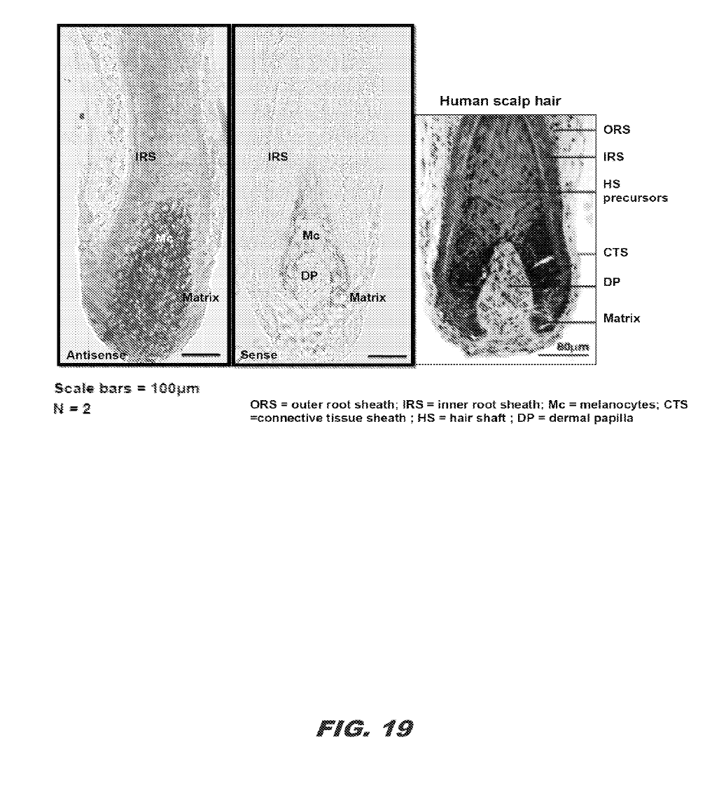

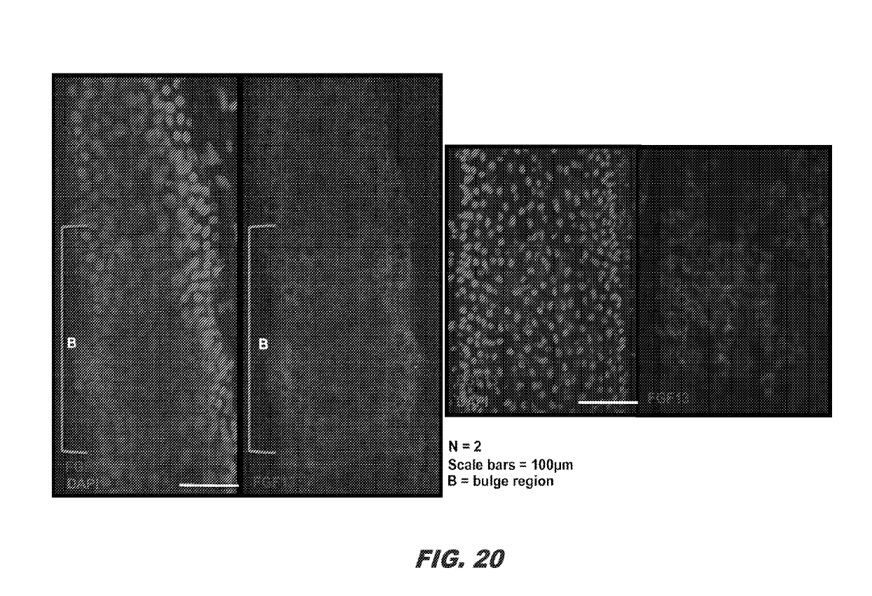

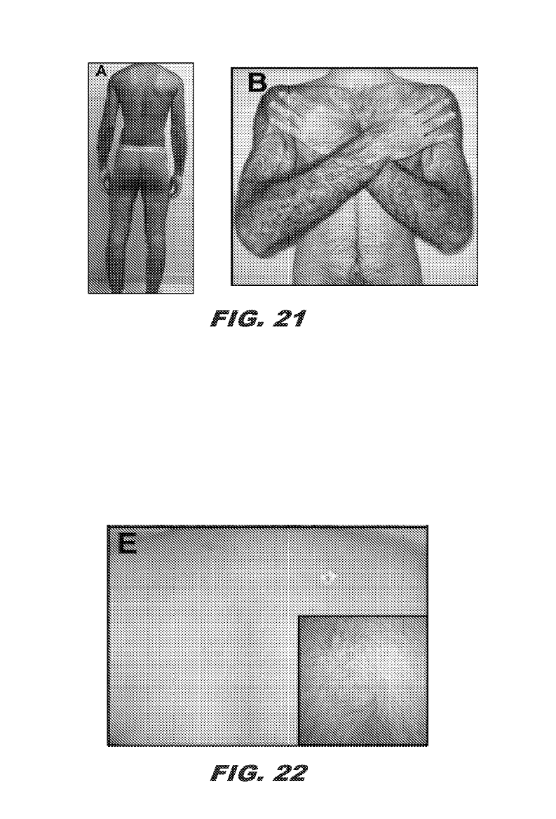

A large kindred from Mexico was ascertained with X′-linked congenital generalized hypertrichosis (CGH) (OMIM 307150) co-segregating with deafness, dental, and palate anomalies (FIGS. 4B-D, FIGS. 9C-D, FIG. 21-22) (P11). Affected males have approximately three times the number of normal hairs on the scalp and exhibit excessive growth of highly pigmented terminal hairs (medullated) on the scalp, back, shoulders, chest, arms, legs as well as on the face (FIGS. 4B-D, FIGS. 9C-D, FIG. 21), whereas hemizygous carrier females have mild hypertrichosis uniformly distributed across the body (FIG. 22).

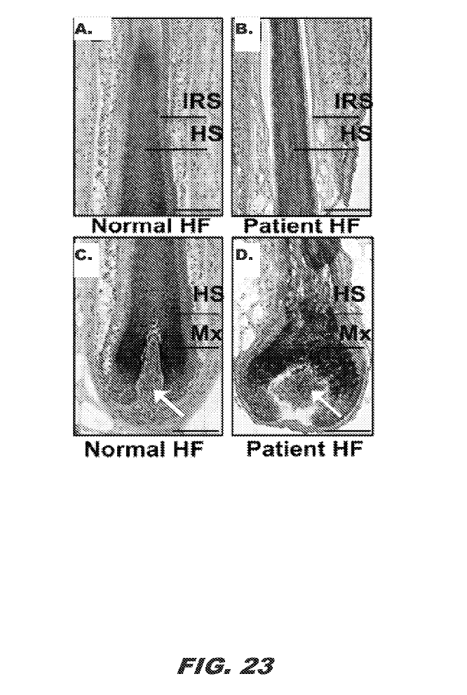

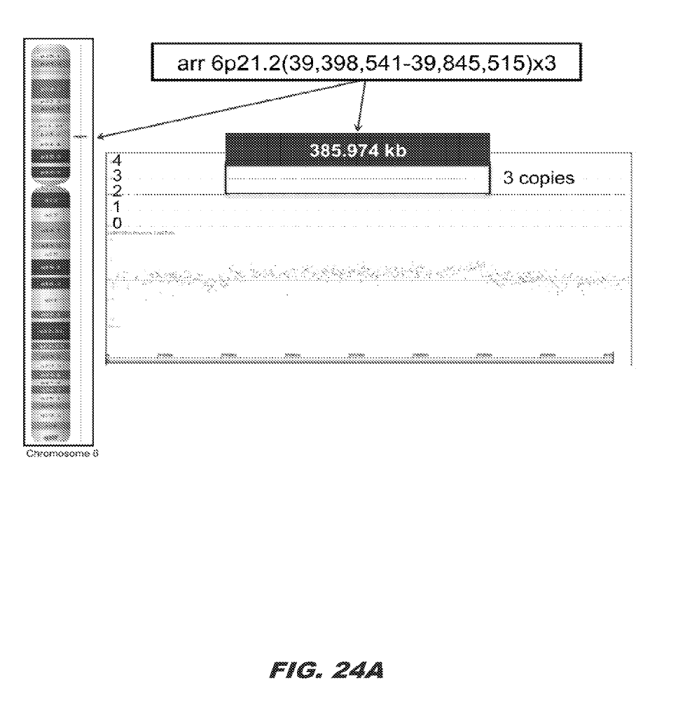

Histological analysis of affected hair follicles using hematoxylin and eosin staining confirmed that the hairs are of the terminal type, since they are medullated, pigmented and penetrate deep within the dermis (FIG. 23B, D). Affected individuals have an increased density in the number of hair follicles as well as a transformation from vellus (fine, non-medullated, unpigmented) to terminal hair follicles on multiple body sites, which cause an excessive hair overgrowth phenotype. Morphometric analysis of affected hair follicles revealed a widened dermal papilla (3-fold increase; p=0.0000343), matrix (1.9-fold increase; p=0.0000642), and hair shaft (1.25-fold increase; p=0.036) in hair follicles from three affected individuals compared to controls (FIG. 23A,C; FIG. 28).

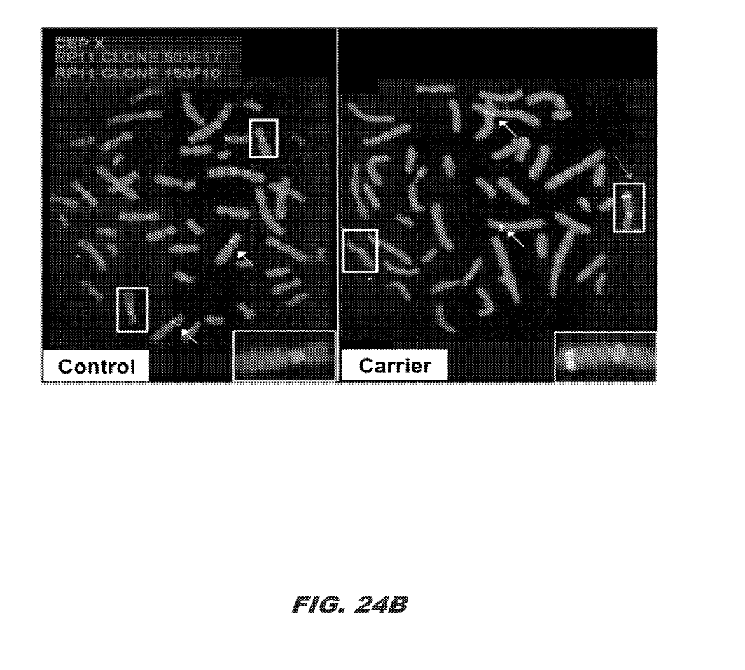

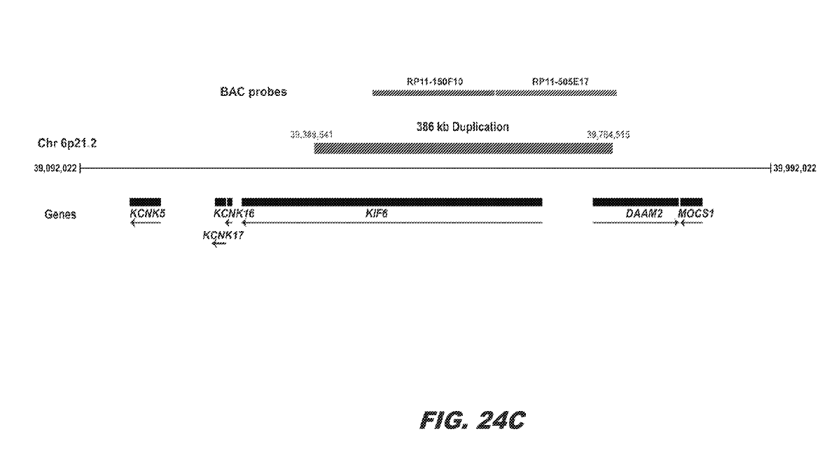

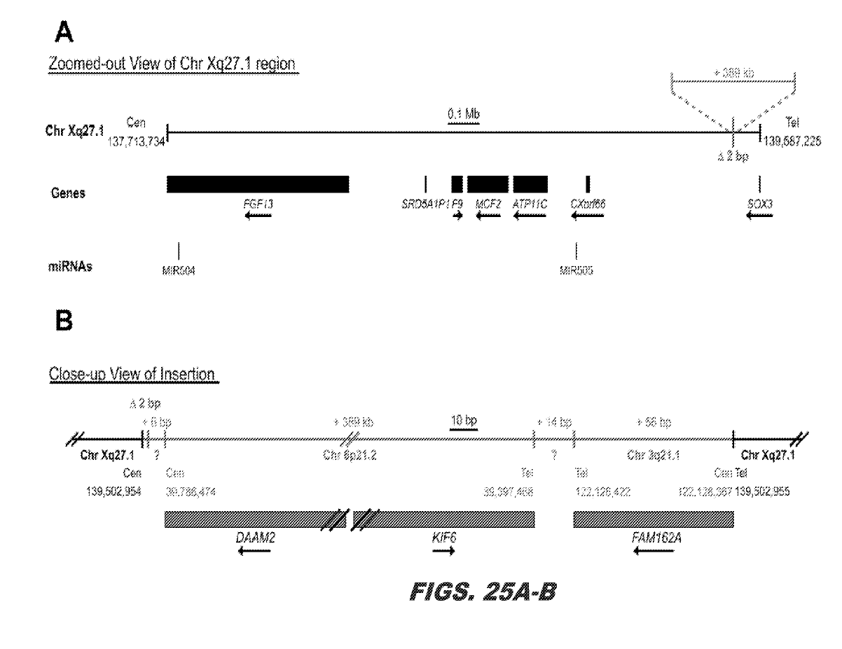

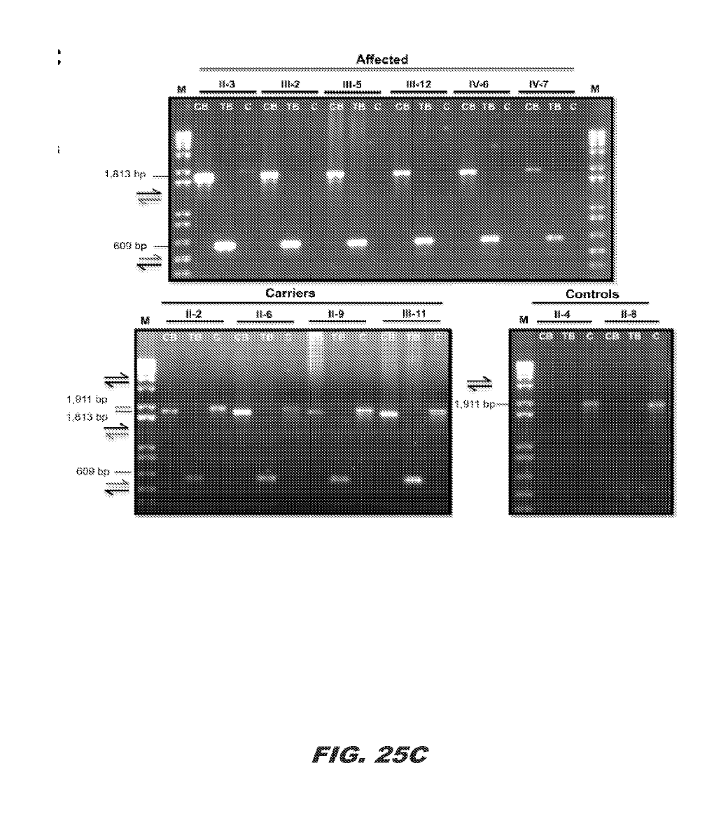

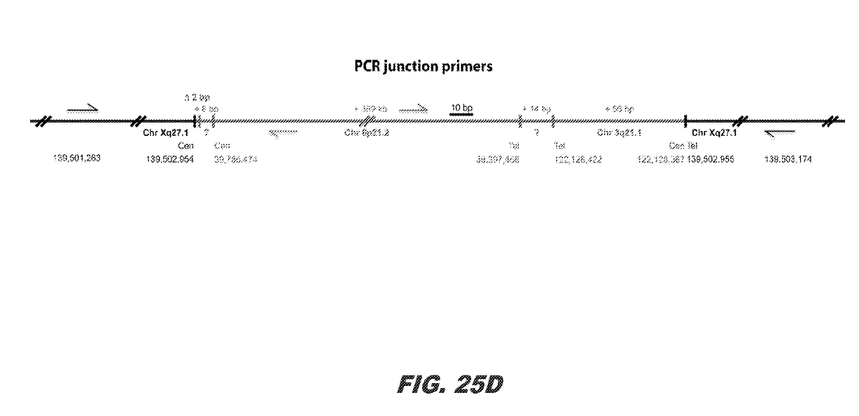

In previous work on this family, linkage analysis was performed, which defined a 19 Mb region on Xq24-27 that spans approximately 82 genes and completely co-segregates with the disease (P11). Sequencing the coding exons of every gene in the interval proved unsuccessful in identifying the mutation. To identify the genetic defect, SNP oligonucleotide microarray analysis (SOMA) was next performed using the Affymetrix Cytogenetics Whole-Genome 2.7M array, which revealed a 386 kb duplication of chromosome 6p21.2 (FIG. 24A). The insertion was then visualized at the cytogenetic level using fluorescence in situ hybridization (FISH) with two non-overlapping BAC probes spanning the chromosome 6p21.2 duplication, which revealed a third signal for chromosome 6 present on the X chromosome (FIG. 24B-C). To identify the insertion breakpoints as well as its content, whole-genome sequencing (WGS) was performed, which revealed a large interchromosomal insertion at an extragenic palindromic sequence on chromosome Xq27.1, consisting of a 386 kb duplication of chromosome 6p21.2 and 56 bp of chromosome 3q21.1 in the reverse orientation, separated by 14 bp of unknown origin (FIG. 25A-B). This complex insertion leads to a two base-pair deletion within the Xq27.1 palindromic site and contains sequences from two genes on chromosome 6p21.2 (DAAM2 and KIF6) and one gene from chromosome 3q21.2 (FAM162A), none of which have reported functions in the skin or hair follicle. To confirm that the insertion co-segregated with the disease phenotype, PCR amplification of the centromeric and telomeric breakpoint junctions on genomic DNA from control, carrier, and affected individuals was used, which revealed junction bands present only in affected and carrier individuals, whereas DNA from controls produced an amplicon representative of an unaffected X chromosome (FIG. 25C-D).

To gain insight into whether the insertion at Xq27.1 affects X-chromosome inactivation in female carriers, as skewed X inactivation is a phenomenon reported in several X-linked disorders (P12, P13), the XCI assay was performed and did not observe significant skewing (>80%) in four out of five carriers (Table 2 and Materials and Methods).

TABLE 2
X chromosome inactivation experiment in XLH female carriers.

| Sample ID | Description | % Skewing |
|---|---|---|
| II-2 | Female carrier | 43% |
| II-9 | Female carrier | 81% |
| II-1 | Female carrier | 67% |
| II-6 | Female carrier | 62% |
| III-11 | Female carrier | 74% |
| II-8 | Female non-affected | Non-informative |
| III-8 | Female non-affected | Non-informative |

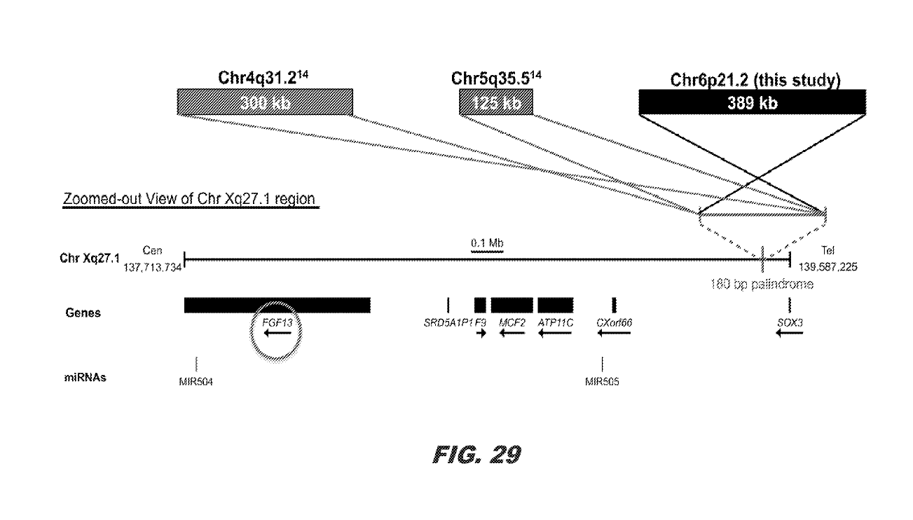

Importantly, two other recently reported families with X-linked congenital generalized hypertrichosis contain linkage to chromosome Xq24-27, consistent with these findings, in which one family contains a 300 kb interchromosomal insertion from chromosome 4q31.2, and the other family contains a 125 kb interchromosomal insertion from chromosome 5q35.3 (P14) (FIG. 29). Interestingly, the insertion events in all three families occur at the same human-specific extragenic palindrome sequence at Xq27.1, indicating that the presence of the insertion (rather than its content) may be responsible for the excessive hair overgrowth phenotype by disruption of the chromosomal architecture in the region.

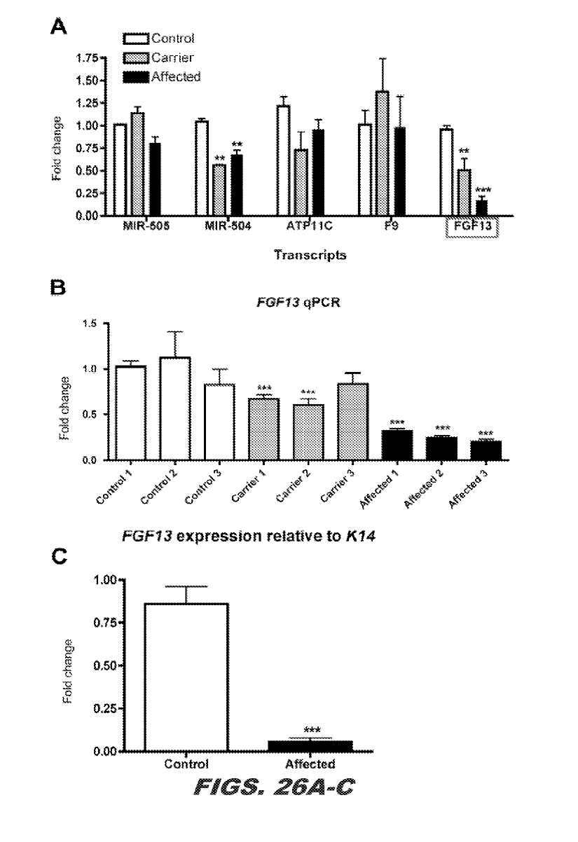

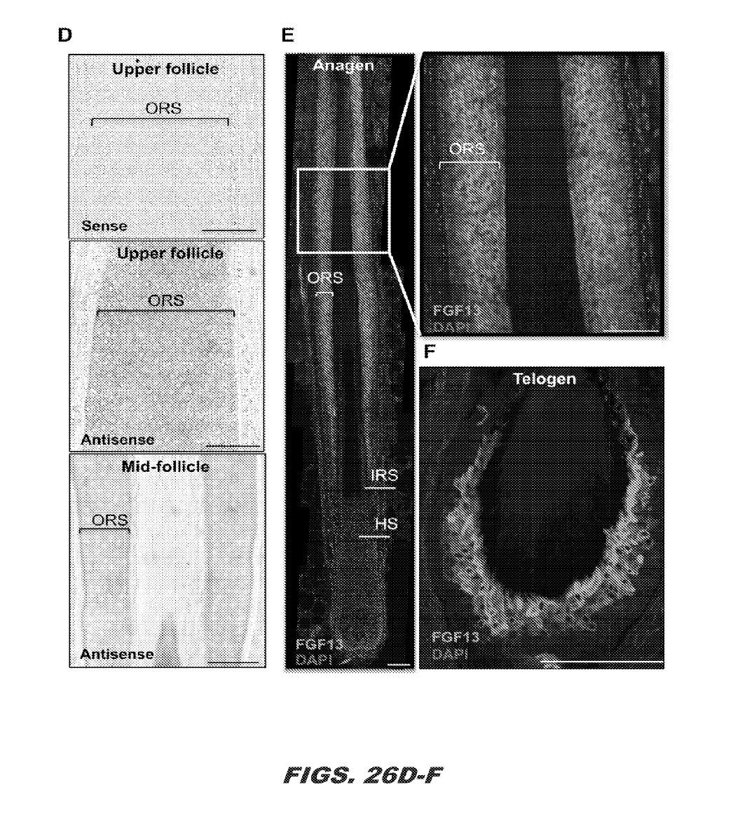

Despite the identification of different insertions in these three cases, their impact on the expression of the surrounding genes has not been thoroughly investigated. Therefore, the expression of several neighboring genes was analyzed using quantitative RT-PCR (qRT-PCR) on RNA isolated from control, carrier and affected skin biopsies. Unexpectedly, FGF13 levels were significantly reduced in affected individuals by approximately 4-fold (p=0.0007) relative to controls and observed a clear dosage effect when comparing levels between carrier and affected individuals (FIG. 26A-B). To verify that the change in FGF13 expression observed in affected skin biopsies was not due to differences in the number of hair follicle cells present, FGF13 expression was normalized to that of KRT14, which marks the outer root sheath and basal layer of the epidermis, and FGF13 levels were dramatically reduced (18.1-fold; p=0.00006) in affected hair follicle cells (FIG. 26C).

Importantly, the mRNA levels of additional neighboring genes were not significantly changed, and SOX3 and Cxorf66 expression levels were undetectable in control and affected individuals (FIG. 26A). To further examine the expression levels of the genes surrounding the 389 kb insertion, RNA sequencing (RNA-seq) was performed on RNA from the skin of one control and one affected individual, which verified the decrease in FGF13 expression (8-fold; p=0.012) and revealed that the expression levels of most of the genes in the surrounding region over a distance of ˜3 Mb on either side of the insertion were not significantly changed (Table 3 and Materials and Methods).

TABLE 3
Differential expression of genes ~3 Mb on either
side of the 389 kb insertion at Xq27.1 by RNA-seq

| | | | In (fold | Fold | |
|---|---|---|---|---|---|
| | | Detectable | change | change | |
| Gene Name | Locus | Expression | A v. U) | (A v. U) | P value |
| ZIC3 | chrX: 136476011-136481925 | NO | N.D. | N.D. | 0.213 |
| LOC158696 | chrX: 137524557-137527465 | NO | N.D. | N.D. | 0.159 |
| FGF13 | chrX: 137541399-138114851 | YES | 2.076 | −7.976 | 0.012 |
| MIR504 | chrX: 137541399-138114851 | NO | N.D. | N.D. | 1.000 |
| LOC100129662 | chrX: 137541399-138114851 | NO | N.D. | N.D. | 1.87E−05 |
| Pseudogene | chrX: 138356643-138358798 | YES | 1.562 | −4.770 | 0.005 |
| (SRD5A1P1) | | | | | |
| F9 | chrX: 138440560-138473283 | NO | N.D. | N.D. | 0.023 |
| MCF2 | chrX: 138491595-138618047 | YES | 0.320 | −1.376 | 0.501 |
| ATP11C | chrX: 138636170-138742113 | YES | 0.184 | −1.201 | 0.663 |
| MIR505 | chrX: 138833972-138834056 | NO | N.D. | N.D. | 1.000 |
| CXorf66 | chrX: 138865549-138875343 | NO | N.D. | N.D. | 1.000 |
| | Insertion at | | | | |
| | chrX: 139,330,583 | | | | |
| SOX3 | chrX: 139412817-139414891 | NO | N.D. | N.D. | 1.000 |
| RP1- | chrX: 139619589-139624662 | NO | N.D. | N.D. | 0.677 |
| 177G6.2 | | | | | |
| CDR1 | chrX: 139693090-139694389 | YES | 0.567 | −1.763 | 0.387 |
| MIR320D2 | chrX: 139836002-139836050 | NO | N.D. | N.D. | 1.000 |
| SPANXB1 | chrX: 139924426-139925542 | NO | N.D. | N.D. | 1.000 |
| LDOC1 | chrX: 140097596-140098976 | YES | 0.382 | −1.465 | 0.484 |
| SPANXC | chrX: 140163261-140164312 | NO | N.D. | N.D. | 1.000 |
| SPANXA1 | chrX: 140418508-140565735 | NO | N.D. | N.D. | 1.000 |
| SPANXA2 | chrX: 140418508-140565735 | NO | N.D. | N.D. | 1.000 |
| SPANXA2- | chrX: 140418508-140565735 | NO | N.D. | N.D. | 0.079 |
| OT1 | | | | | |
| SPANXD | chrX: 140613233-140614321 | NO | N.D. | N.D. | 1.000 |
| SPANXE | chrX: 140613233-140614321 | NO | N.D. | N.D. | 1.000 |
| MAGEC3 | chrX: 140753767-140813284 | NO | N.D. | N.D. | 0.159 |
| MAGEC1 | chrX: 140819307-140824853 | NO | N.D. | N.D. | 1.000 |
| MAGEC2 | chrX: 141117793-141120742 | NO | N.D. | N.D. | 1.000 |
| SPANXN4 | chrX: 141941369-141949732 | NO | N.D. | N.D. | 1.000 |

* A = affected; U = unaffected; ln = natural logarithm; N.D. = not detectable

The expression of miR-504, intronic to FGF13, was reduced by approximately 1.5-fold in the carrier and affected individuals by qRT-PCR (p=0.0121) (FIG. 26A). FGF13 is not a predicted target gene of miR-504, yet there are several predicted targets with known roles in hair follicle development and cycling whose expression levels were altered in X-linked hypertrichosis, detected by RNA-sect (Table 4 and Materials and Methods). While these changes in gene expression may simply reflect a difference in the number of hair follicle cells present, without being bound by theory, the reduction of FGF13-miR-504 transcripts either directly or indirectly leads to increased expression of some of these downstream genes.

TABLE 4
miR-504 predicted target genes differentially expressed in XLH by RNAseq.

| | | Fold | | | |
|---|---|---|---|---|---|
| | | change | | | |
| Gene | | (Affected vs | | | |
| Name | Locus | Unaffected) | ln(FC) | p_value | q_value |
| BATF2 | chr11: 64511992-64521093 | 40.711 | −3.707 | 0.001 | 0.008 |
| FZD9 | chr7: 72486044-72488386 | 34.025 | −3.527 | 1.80E−05 | 0.000 |
| TGM4 | chr3: 44891101-44931092 | 31.450 | −3.448 | 0.002 | 0.015 |
| NPTX1 | chr17: 76055227-76064999 | 30.048 | −3.403 | 3.92E−09 | 2.91E−07 |
| CRTAM | chr11: 122214464-122248557 | 26.236 | −3.267 | 0.003 | 0.022 |
| HTR7 | chr10: 92490555-92607651 | 22.499 | −3.113 | 0.000 | 0.002 |
| UBASH3B | chr11: 122031607-122190397 | 21.982 | −3.090 | 2.47E−08 | 1.49E−06 |
| FUT5 | chr19: 5816836-5821551 | 19.225 | −2.956 | 2.66E−05 | 0.001 |
| ATP8B3 | chr19: 1733073-1763270 | 18.705 | −2.929 | 2.83E−08 | 1.67E−06 |
| MDGA1 | chr6: 37708261-37773744 | 16.793 | −2.821 | 6.99E−07 | 2.79E−05 |
| FJX1 | chr11: 35596310-35598997 | 11.675 | −2.457 | 5.67E−06 | 0.000 |
| KIF21B | chr1: 199205136-199259451 | 11.648 | −2.455 | 6.60E−13 | 9.76E−11 |
| MET | chr7: 116099694-116225676 | 10.666 | −2.367 | 1.44E−06 | 5.17E−05 |
| TP53RK | chr20: 44619868-44751683 | 10.417 | −2.343 | 4.26E−05 | 0.001 |
| MALL | chr2: 110198735-110231432 | 10.377 | −2.340 | 1.09E−05 | 0.000 |
| IL6R | chr1: 152644292-152708550 | 10.361 | −2.338 | 1.09E−08 | 7.38E−07 |
| STC1 | chr8: 23755378-23768265 | 10.269 | −2.329 | 1.39E−05 | 0.000 |
| HAS3 | chr16: 67696967-67723994 | 9.828 | −2.285 | 1.78E−08 | 1.13E−06 |
| S100A7A | chr1: 151655623-151662325 | 8.701 | −2.163 | 3.66E−05 | 0.001 |
| MTHFD2 | chr2: 74279197-74295932 | 8.430 | −2.132 | 2.00E−06 | 6.96E−05 |
| CDCP1 | chr3: 45098772-45162918 | 8.277 | −2.113 | 5.62E−05 | 0.001 |
| EXOSC6 | chr16: 68841634-68843334 | 8.037 | −2.084 | 0.000 | 0.002 |
| TNFRSF9 | chr1: 7898517-7925812 | 8.020 | −2.082 | 0.004 | 0.030 |
| P2RY11 | chr19: 10077898-10091599 | 7.800 | −2.054 | 0.000 | 0.002 |
| LPCAT1 | chr5: 1514541-1577076 | 7.264 | −1.983 | 0.000 | 0.003 |
| LIF | chr22: 28966441-28972796 | 6.932 | −1.936 | 0.000 | 0.003 |
| PNO1 | chr2: 68238508-68256595 | 6.650 | −1.895 | 0.000 | 0.005 |
| BYSL | chr6: 41996942-42008762 | 5.907 | −1.776 | 0.001 | 0.009 |
| TRIB3 | chr20: 309307-326203 | 5.706 | −1.742 | 0.001 | 0.010 |
| PCDH10 | chr4: 134289919-134332182 | 5.591 | −1.721 | 0.005 | 0.033 |
| BCAT1 | chr12: 24854224-24993660 | 5.357 | −1.678 | 0.001 | 0.010 |
| CNO | chr4: 6768742-6770288 | 5.247 | −1.658 | 0.003 | 0.023 |
| CYGB | chr17: 72035024-72053053 | 5.226 | −1.654 | 0.005 | 0.035 |
| ZFAT | chr8: 135559212-135794474 | 5.216 | −1.652 | 8.57E−08 | 4.52E−06 |
| STK40 | chr1: 36577811-36624072 | 5.210 | −1.651 | 0.002 | 0.016 |
| SPRED2 | chr2: 65391488-65513160 | 5.123 | −1.634 | 9.36E−05 | 0.002 |
| FUT2 | chr19: 53891039-53901003 | 5.119 | −1.633 | 0.000 | 0.005 |
| IPPK | chr9: 94099460-94472368 | 5.105 | −1.630 | 0.002 | 0.015 |
| PXN | chr12: 119123476-119187957 | 5.053 | −1.620 | 5.16E−08 | 2.90E−06 |
| CD86 | chr3: 123256898-123322678 | 5.052 | −1.620 | 0.001 | 0.007 |
| ABL2 | chr1: 177335084-177465442 | 5.027 | −1.615 | 9.74E−13 | 1.43E−10 |
| KLHL21 | chr1: 6573370-6585516 | 4.985 | −1.606 | 0.007 | 0.044 |
| FSCN1 | chr7: 5598961-5612813 | 4.963 | −1.602 | 0.004 | 0.031 |
| RUNX1 | chr21: 35081967-35343465 | 4.835 | −1.576 | 2.14E−05 | 0.001 |
| RRAGC | chr1: 39077605-39097927 | 4.730 | −1.554 | 0.002 | 0.020 |
| SERTAD1 | chr19: 45620248-45623772 | 4.697 | −1.547 | 0.003 | 0.022 |
| OSBP2 | chr22: 29420792-29633811 | 4.695 | −1.547 | 0.005 | 0.033 |
| ZNF264 | chr19: 62394679-62426026 | 4.653 | −1.538 | 0.004 | 0.028 |
| DNMBP | chr10: 101625323-101759666 | 4.581 | −1.522 | 0.003 | 0.023 |
| PLEKHM1 | chr17: 40869048-40923929 | 4.558 | −1.517 | 2.80E−05 | 0.001 |
| PDRG1 | chr20: 29996418-30003544 | 4.414 | −1.485 | 0.007 | 0.042 |
| MBP | chr18: 72819776-72973762 | 4.300 | −1.459 | 3.75E−05 | 0.001 |
| PLCD3 | chr17: 40544533-40565417 | 4.266 | −1.451 | 0.005 | 0.033 |
| SLC7A1 | chr13: 28981550-29067825 | 4.162 | −1.426 | 0.007 | 0.043 |
| GMEB2 | chr20: 61689398-61728825 | 4.160 | −1.425 | 0.007 | 0.041 |
| LRRC8A | chr9: 130684211-130720138 | 4.158 | −1.425 | 0.000 | 0.006 |
| KCNK1 | chr1: 231816372-231874881 | 4.037 | −1.396 | 0.006 | 0.039 |
| NUDT15 | chr13: 47509703-47519283 | 4.004 | −1.387 | 0.008 | 0.046 |
| LY6K | chr8: 143778530-143805393 | 3.958 | −1.376 | 0.001 | 0.006 |
| SMURF1 | chr7: 98462993-98579679 | 3.939 | −1.371 | 0.002 | 0.016 |
| GPER | chr7: 1003148-1144419 | 3.871 | −1.354 | 0.008 | 0.047 |
| RRAS2 | chr11: 14256041-14342628 | 3.637 | −1.291 | 0.000 | 0.002 |
| CRTC3 | chr15: 88874201-88989581 | 3.559 | −1.270 | 0.001 | 0.010 |
| MAPKAP | chr1: 204924987-204974253 | 3.464 | −1.242 | 0.005 | 0.035 |
| K2 | | | | | |
| ZBTB24 | chr6: 109890411-109911133 | 3.342 | −1.207 | 0.008 | 0.047 |
| NRF1 | chr7: 129038790-129184158 | 3.334 | −1.204 | 0.006 | 0.038 |
| DIAPH1 | chr5: 140874771-140978806 | 3.312 | −1.197 | 0.001 | 0.011 |
| PAPD5 | chr16: 48744329-48826720 | 3.160 | −1.151 | 0.008 | 0.045 |
| DHX33 | chr17: 5284955-5313104 | 3.083 | −1.126 | 0.003 | 0.022 |
| PVR | chr19: 49838937-49861268 | 2.872 | −1.055 | 0.001 | 0.009 |
| TGFBR1 | chr9: 100907232-100956294 | 2.854 | −1.049 | 0.008 | 0.046 |
| GAS7 | chr17: 9754650-10042593 | 2.703 | −0.994 | 0.006 | 0.036 |
| IL1RAP | chr3: 191714533-191857680 | 2.632 | −0.968 | 0.002 | 0.015 |
| CTDP1 | chr18: 75540788-75615498 | 2.594 | −0.953 | 0.007 | 0.044 |
| VEGFA | chr6: 43845923-43862201 | 2.490 | −0.912 | 0.000 | 0.005 |
| CNOT4 | chr7: 134697086-134845415 | 2.220 | −0.798 | 0.004 | 0.026 |

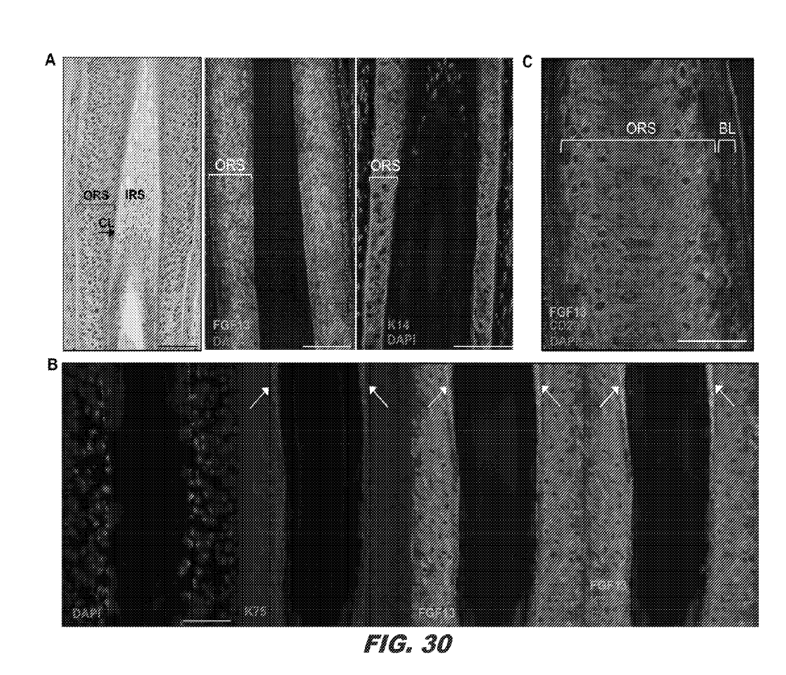

FGF13 is a plausible candidate gene to be the target of the position effect, since its expression was previously detected in the hair follicle bulge, which is the stem cell compartment of the hair follicle (P15, P 16). However, it was unclear whether these were the only FGF13-expressing cells in the human hair follicle or whether expression was more widespread. Using in situ hybridization and immunofluorescence staining on hair follicles in the growth stage of the hair cycle, anagen, expression of FGF13 was detected in the outer root sheath within the middle and upper portions of the human hair follicle (FIG. 26D-E). FGF13 expression was also observed in the trichilemma, or outer root sheath compartment of the club hair during the resting stage of the hair cycle, telogen, by immunofluorescence staining (FIG. 26F). In anagen hairs, FGF13 localizes to the ORS where KRT14 is expressed, and also localizes to the companion layer that separates the outer from inner root sheath, as evidenced by overlapping expression with KRT75 (companion layer marker) (FIG. 30A-B). However, FGF13 did not localize to the bulge region of the anagen human hair follicle, which was observed through co-staining with CD200, a well-characterized bulge marker (FIG. 30C).

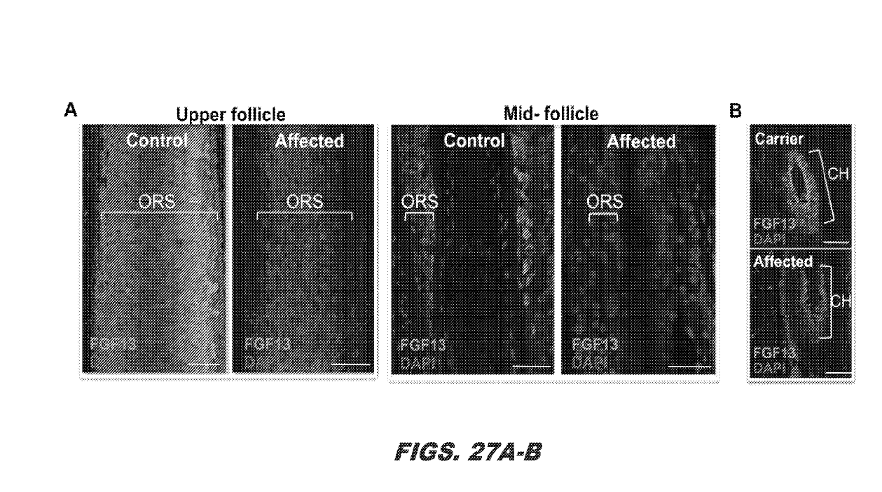

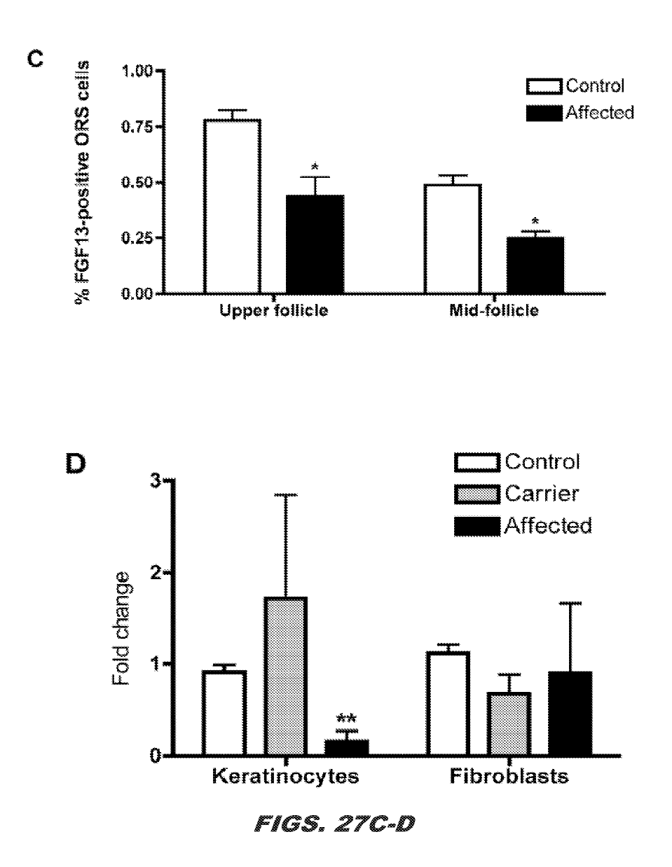

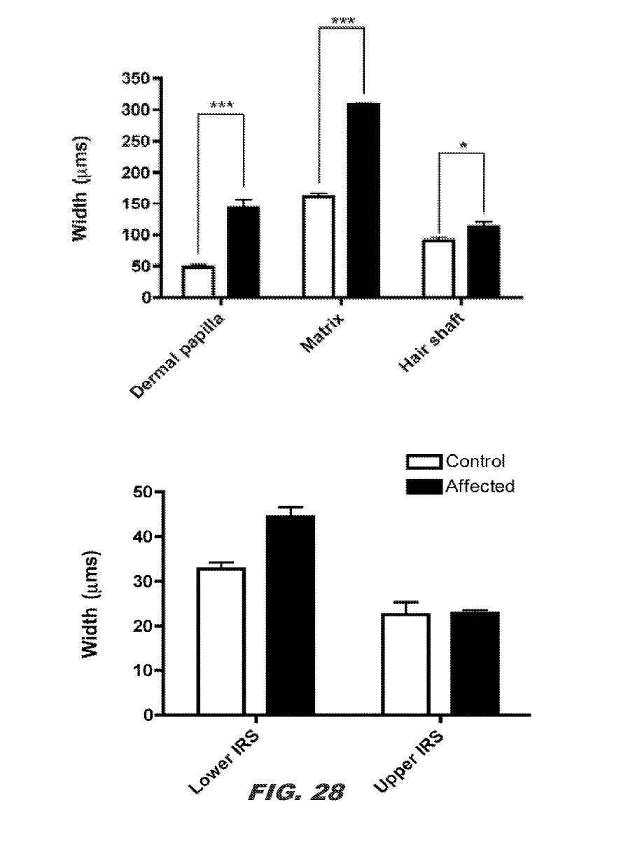

To gain insight into which cells exhibited decreased FGF13 levels, FGF13 immunofluorescence staining was performed on control, carrier and affected hair follicles, and a decrease in the intensity of expression and number of FGF13-positive cells was observed throughout the outer root sheath of affected anagen follicles compared with controls and carriers (FIG. 27A, C). Moreover, a comparison of affected and carrier telogen follicles revealed a decrease in expression throughout the affected hair follicle, recapitulating the dosage effect observed at the mRNA level (FIG. 25B).

To further investigate the selective decrease of FGF13 expression in affected keratinocytes, qRT-PCR was performed on keratinocytes and fibroblasts cultured from control, carrier, and affected skin biopsies, and observed a significant decrease in FGF13 expression in affected keratinocytes by 6.7-fold (p=0.0185) (FIG. 25D), but not the fibroblasts. Consistent with previous observations, findings localize the defect to the keratinocyte compartment.

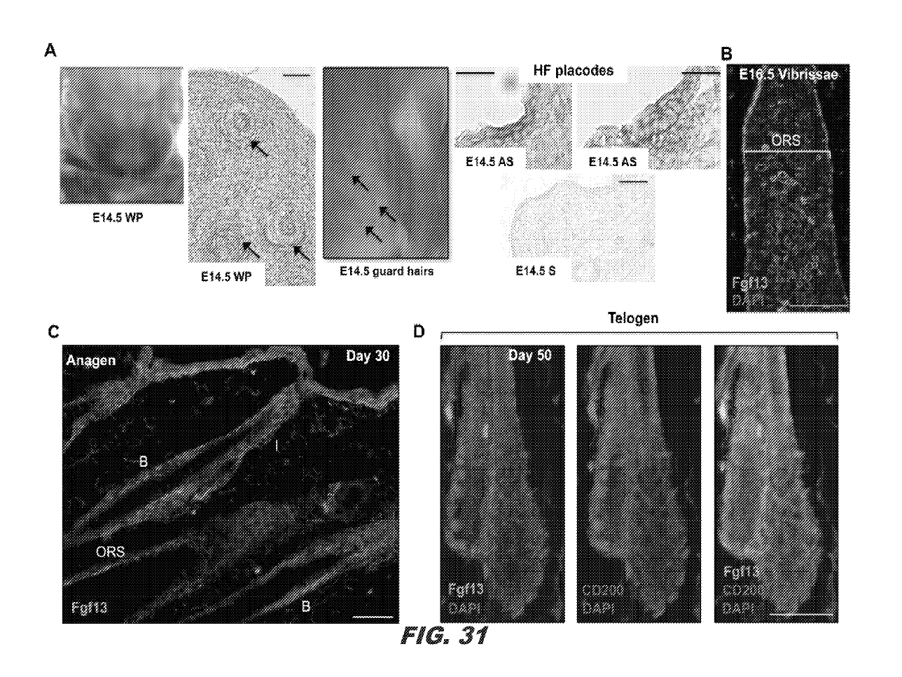

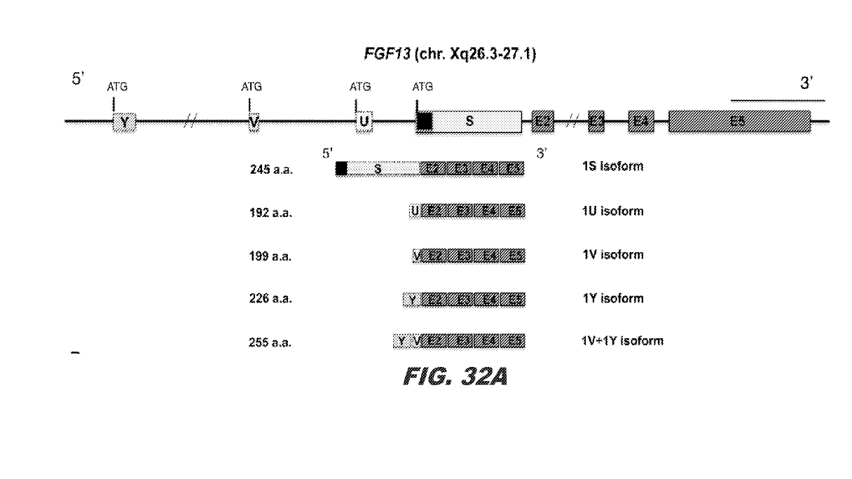

To determine Fgf13 expression during murine hair follicle morphogenesis, whole-mount and section in situ hybridization was performed on E12.5-E16.5 embryos and observed strong expression in placodes (the sites of newly-forming follicles), as well as within the dermal condensate beneath the placodes that will become the future dermal papilla of the hair follicle, at E14.5 within the developing whisker pad and guard hair pelage follicles (FIG. 31A). Immunofluorescence staining of vibrissae follicles during morphogenesis at E16.5 revealed that Fgf13 localizes to the outer root sheath, similar to the postnatal localization pattern of the human FGF13 protein in anagen follicles (FIG. 31B). Moreover, immunofluorescence staining on postnatal skin revealed that Fgf13 localizes to the bulge, isthmus region, and outer root sheath of the hair follicle (FIGS. 31C-D). Without being bound by theory, Fgf13 plays a role in regulating hair follicle growth and cycling.

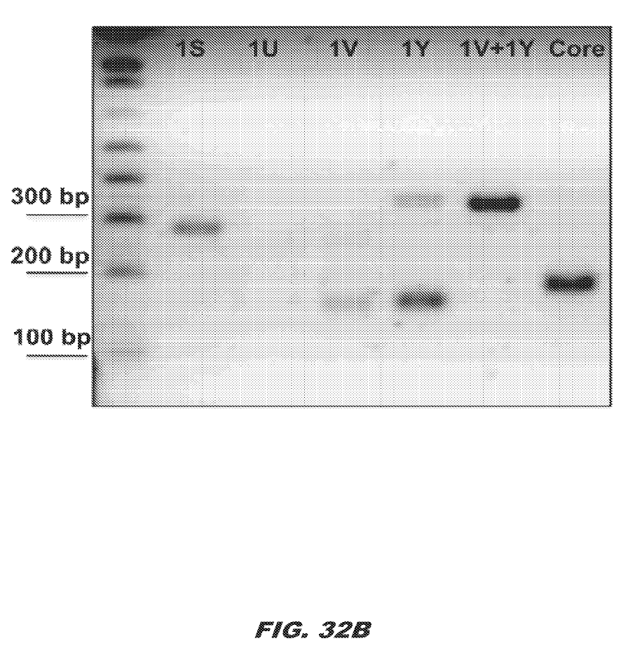

In mice and in humans, five-prime alternative splicing of FGF13 and usage of different transcription start sites generates transcripts with distinct 5′ exons referred to as 1 S, 1U, 1V, 1Y and 1V+1Y, where exons 2-5, encoding the conserved core region of the protein, are common to all transcripts. Isoform-specific PCR revealed that isoforms 1 S, 1V, 1Y, and 1V+1Y are expressed in human scalp skin (FIG. 32). To gain insight into the mechanism by which the interchromosomal insertion alters FGF13 transcript levels in X-linked hypertrichosis, the RNA-seq data was utilized to test for differentially expressed isoforms using Cuffdiff (see Materials and Methods), but differential expression was not observed between the FGF13 isoforms, indicating that the interchromosomal insertion disrupts the transcription of all isoforms rather than altering the usage of a particular transcription start site.

Position effects on single genes have been reported in several other human genetic diseases associated with large chromosomal rearrangements, where the distances of the furthest breakpoints from the target genes were as large as 1.0 Mb (SHH in preaxial polydactylyl II) (P17, P18), 1.3 Mb (SOX9 in camptomelic dysplasia) (P19, P20) and 7.3 Mb (TRPS1 in Ambras syndrome) (P8). Since FGF13 lies 1.2 Mb away from the insertion and its expression was selectively reduced, the interchromosomal insertion at Xq27.1 separates the gene from a tissue- or temporal-specific modifier element (such as an enhancer) required for proper FGF13 expression during hair follicle morphogenesis and cycling. FGF13 expression was selectively reduced in a tissue-specific manner, as transcript levels were decreased in affected keratinocytes, but not fibroblasts.

The data indicate that FGF13 is primarily expressed throughout the outer root sheath of human hair follicles, and findings from the clinical, histological and morphometric analyses revealed increased width of hair follicles in X-linked hypertrichosis (FIG. 23B, D; FIG. 28). Several genes expressed in the outer root sheath of hair follicles have been reported to have non-cell autonomous effects on other compartments, indirectly or directly leading to changes in hair follicle width and/or length. In the case of Dishevelled 2 (Dvl2), an effector of Wnt3 signaling normally expressed in the outer root sheath, precortical and precuticle cells of the hair shaft, its overexpression in the outer root sheath induces a short hair phenotype by altering the differentiation of hair shaft precursor cells (P21). Similarly, overexpression of Vegf in the outer root sheath, where it is normally expressed, induces perifollicular vascularization of the hair follicle, resulting in accelerated hair regrowth and well as increased size of hair shafts (P22).

In mouse expression studies, Fgf13 is expressed during hair follicle induction and morphogenesis, indicating it may play an important role in these processes. Affected individuals in the X-linked hypertrichosis family possess an increased density of hairs, thus, dysregulation of FGF13 levels in X-linked hypertrichosis leads to the formation of extra hairs. Further studies using Fgf13-deficient mice would directly implicate a role for Fgf13 in hair follicle morphogenesis and cycling. Interestingly, Fgf13 expression has been demonstrated in the dental mesenchyme and developing tooth bud (P23), an additional site of pathology for XLH patients who have dental and palate anomalies, suggesting a potential role for this gene in odontogenesis.

Several FGFs and their receptors are known to play important roles in hair regrowth. While canonical FGFs signal through their respective receptors to control hair growth via stem cell activation and quiescence, FGF13 is the first non-canonical FGF to be implicated in hair follicle morphogenesis and cycling. As FGF13 has been reported to bind the MAPK scaffolding protein IB2 (P24), leading to activation of a stress-induced MAPK that lies downstream of the canonical FGF signaling pathway (P24), without being bound by theory, FGF13 internally modulates the transcriptional output of canonical FGF signaling to control hair growth. Expression levels of several FGFs are dysregulated in X-linked hypertrichosis (Table 5).

TABLE 5
FGFs with dysregulated expression levels
in XLH by RNA-seq in whole skin.

| | | Fold change | |
|---|---|---|---|
| | | (patient vs. | |
| Gene Name | Locus | control) | P value |
| FGF5 | chr4: 81406765-81431195 | −56.33 | 2.53E−09 |
| FGF18 | chr5: 170779271-170817235 | −11.22 | 3.00E−05 |
| FGF14 | chr13: 101171205-101852125 | −9.13 | 0.0004 |
| FGF13 | chrX: 137541399-138114851 | −7.98 | 0.012 |
| FGF12 | chr3: 193339875-193928082 | −6.09 | 0.006 |
| FGFR1OP | chr6: 167332805-167374056 | −3.37 | 0.007 |
| FGFR3 | chr4: 1764836-1780397 | −3.21 | 0.001 |
| FGFR2 | chr10: 123227833-123347962 | −2.32 | 0.0008 |
| FGFBP1 | chr4: 15546289-15549461 | 13.58 | 3.70E−06 |

Among these was FGF5, a known regulator of the anagen-to-catagen transition and responsible for the excessive hair overgrowth phenotype in angora mice, dogs, goats, and rabbits (P25-P28). Without being bound by theory, FGF13 can also act as a microtubule stabilizing protein in the hair follicle, similar to its role in neurons (P29), to regulate additional signaling molecules active in the developing follicle. Further functional studies on FGF13 will reveal the mechanism by which it regulates hair follicle growth and distribution.

In this Example, a 389 kb interchromosomal insertion at Xq27.1 was identified that completely co-segregates with the X-linked CGH phenotype, and among the genes surrounding the insertion, FGF13 expression was selectively and profoundly reduced. FGF13 lies 1.2 Mb away from the insertion, revealing a position effect on a distant gene as a result of the chromosomal insertion at Xq27.1. Although these large interchromosomal insertions can mediate pathogenic effects by introducing new regulatory elements, the presence of the insertions (rather than their content) is responsible for the hair overgrowth phenotype since the sequences contained within each insertion are different among the families (P14). Moreover, these insertions occur at an extragenic palindrome sequence and do not disrupt the coding region of a gene in the surrounding region (FIG. 29).

The density of hair follicles covering the human body is markedly reduced compared to other primates, and as such, the excessive hair phenotype observed in hereditary hypertrichosis is reminiscent of an atavism (P3). Various examples of atavisms involving several body parts have been reported in mammals, including the occurrence of complete ulnas and fibulas in miniature horses, reptile-like coronary circulation and myocardial architecture in humans, and the development of the ancestral tooth primordia in retinoic acid receptor-deficient mice (P30-P32). Importantly, the occurrence and prevalence of these ancestral features is indicative of a genetic basis, particularly those involving unusual mechanisms. Here, a position effect on FGF13 in X-linked hypertrichosis is reported that alters the spatio-temporal expression of the gene in the hair follicle. The altered FGF13 expression in affected hair follicles influences important downstream signaling pathways, ultimately leading to the terminal hair overgrowth phenotype of X-linked hypertrichosis.

Materials and Methods

Patient Materials, Histological Analysis, and RNA Extraction.

DNA was previously collected from 26 members of the family, three of whom are obligate carriers and eight of whom are affected (P11). Whole skin biopsies taken from the back were then obtained from three female carriers and three affected male individuals. Biopsies were divided into three separate pieces for RNA extraction, cell culture (see below for details), and OCT embedding for histological and morphometric analysis (see below for details). Control hair follicles from occipital scalp biopsies as discarded tissue was obtained following hair transplant surgeries. RNA extraction was performed using the Qiagen RNeasy Mini Kit following the manufacturer's instructions. Total RNA was used for first-strand cDNA synthesis, as previously described (P8).

SNP Oligonucleotide Microarray Analysis (SOMA) and Whole-Genome Sequencing (WGS).

DNA from an affected individual was prepared and hybridized as per the manufacturers instructions on the Affymetrix Cytogenetics Whole-Genome 2.7M array, and data were analyzed on the Affymetrix® Chromosome Analysis Suite. WGS was performed on one affected male of this Mexican family using the methods described below. All coordinates reference UCSC human reference genome build hg19.

Cytogenetic Analysis, Amplification of Genomic DNA, and qRT-PCR.

FISH analysis was performed on metaphase chromosome spreads prepared from PHA-stimulated cultured peripheral blood cells using standard techniques. The RPCI-11 clone 505E17 (labeled with Orange 5-TAMRA dUTP) and RPCI-11 clone 150F10 (labeled with Green 5-Fluorescein dUTP) from Empire Genomics were used as FISH probes. Hybridization and post-hybridization washing were performed as per the manufacturers instructions. To test co-segregation of the insertion with the disease phenotype, 100 ng of DNA was used for PCR amplification of the centromeric and telomeric breakpoints as well as the control region of the unaffected X chromosome (details listed below). Quantitative RT-PCR was performed as previously described (P8) using the ddCT method and primers listed below.

In Situ Hybridization and Immunofluorescence Staining.

Whole-mount in situ hybridization was performed as previously described (P8) (see below for details). Immunofluorescence staining was performed on human control, carrier, and affected 12 μm hair follicle sections as well as on Swiss Webster dorsal skin sections (10 μm) from postnatal day 30 (anagen) and 50 (telogen) mice using the conditions described below.

Histological and Morphometric Analysis of XLH Skin Biopsies.

Whole skin biopsies from the affected, carrier and control individuals were embedded in OCT and a microtome cryostat was used to create 12 μm-thick hair follicle sections. Sections were stained with hematoxylin and eosin, permanently mounted with Permount (Thermo Fischer Scientific), and imaged using an HRc AxioCam fitted onto an Axioplan2 fluorescence microscope (Carl Zeiss, Thornwood, N.Y., USA). For morphometric analysis, the length-measuring tool in the AxioVision (release 4.8.2) program was used to calculate the distance between two points for each of the indicated hair follicle components; the widest distance for each structure was used and the average value was taken using 2-4 measurements per section (with 3-6 sections per slide). Hair follicles were analyzed from one control and two affected individuals, where each skin biopsy contained two hair follicles, both of which were analyzed.

Isolation and Culture of Human Keratinocytes and Fibroblasts from Whole-Skin Biopsies.

Keratinocytes and fibroblasts were grown from control, carrier, and affected skin biopsies using the following protocol: skin biopsies were collected in 10% BCS in Dulbecco's Modified Eagle Medium (DMEM), washed with 5 ml PBS, and then chopped into small pieces that were transferred to 5 ml Dispase (5 mg/ml) overnight at 4° C. Epidermis and dermis were separated with a scalpel and the epidermis was placed into 5 ml 0.25% trypsin-EDTA at 37° C. for 30 minutes and then into 20 ml 10% FBS in DMEM. Cells were collected by centrifugation at 1000 RPM for 7 minutes and resuspended in epidermal keratinocyte growth media, defined with supplements (CnT-07; CELLnTECH). Fibroblasts were isolated by digesting the dermis in 10 ml 0.3% collagenase for 4 hours at 37° C. Cells were collected by centrifugation at 1200 RPM for 10 minutes, washed in 30 ml fibroblast culture medium (10% FBS in DMEM) twice, and then resuspended in fibroblast culture medium.

Whole-Genome Sequencing (WGS).

DNA was prepared for sequencing according to the Illumina DNA sample preparation kit protocol. Initially, the DNA was randomly fragmented by nebulization followed by end repair, addition of a single A base, adaptor ligation, gel electrophoresis to isolate 300 bp fragments followed by PCR amplification. Next, the size-selected libraries were used for cluster generation on the flow cell. All prepared flow cells were run on the Illumina HiSeq using the paired-end module: the paired-end reads were each 100 bp long. DNA was aligned to the reference genome (NCBI Build 36 Ensemb1 release 50) using the BWA software (version 0.4.9) (R1). Picard was used to remove potential PCR duplicates via the rmdup command. SAMtools (version 0.1.5c) was used for variant identification, using the pileup command with the −c option and default settings (R2). The variants were then filtered using SAMtool's variation filter with the default settings but removing the filter for a maximum allowed coverage per variant by setting it to 10 million. All variants were screened for quality by only keeping those with a consensus score and quality score of at least 20 (50 for INDELs) and that had at least 3 reads supporting the variant. Heterozygous INDELs were also excluded if the ratio of variant reads to reference reads was less than 0.2. The average coverage for this sample was 44.4x. Large deletions and duplications were identified with the Estimation by Read Depth with SNVs (ERRS; http://www.duke.edu/˜mz34/erds.htm) software. Structural variants including insertions and translocations were identified using SV-Finder, software developed in the Duke Center for Human Genome Variation that utilizes multiple alignment-based approaches with an emphasis on split-read and pair-end.

The genornewide identification of functional gene variants was facilitated by SequenceVariantAnalyzer (SVA) (R3).

Amplification of Genomic DNA.

The reaction conditions were as follows: 95° C. for 5 minutes, 94° C. for 30 seconds, 55° C. for 40 seconds, 68° C. for 1.5 minutes, where 35 cycles were run with a final extension time of 10 minutes at 68° C. The primers used for the control reaction were: (F) TGGCATTACAAGAGTTAGCTTCTGA (SEQ ID NO: 22); (R) AATGCTTTGTAGTGGCTTTGTTTCC (SEQ ID NO: 23), producing an amplicon of 1,911 bp (R4); the primers used for the centromeric breakpoint were: (F) TGGCATTACAAGAGTTAGCTTCTGA (SEQ ID NO: 24); (R) CCTCCAGGGTGACTAAATTTG (SEQ ID NO: 25), producing an amplicon of 1,813 bp; and the primers used for the telomeric breakpoint were: (F) AACTAGAAGGCCATTGGCTG (SEQ ID NO: 26); (R) AATGCTTTGTAGTGGCTTTGTTTCC (SEQ ID NO: 27), producing an amplicon of 609 bp.

Quantitative RT-PCR Analysis.

The primers used in these assays were as follows: hFGF13 (core): F: CAGCCGACAAGGCTACCAC (SEQ ID NO: 28), R: GTTCCGAGGTGTACAAGTATCC (SEQ ID NO: 29); hMCF2: F: GCAGCAGGAACTTTTGACAG (SEQ ID NO: 30), R: GCTGGTGTGTTCCAATTCAG (SEQ ID NO: 31); hSOX3: F: GTTGGGACGCCTTGTTTAGC (SEQ ID NO: 32), R: TAGCGCGAAGAAATATCAAACAG (SEQ ID NO: 33) (R4); hF9: F: GCATTCTGTGGAGGCTCTATC (SEQ ID NO: 34), R: GCTGCATTGTAGTTGTGGTG (SEQ ID NO: 35); hATP11C: F: GGACATTTCTGGCTGCCTTTG (SEQ ID NO: 36), R: CCAGAATCGGGTATCCAAG (SEQ ID NO: 37); hK14: F: GGGATCTTCCAGTGGGATCT (SEQ ID NO: 38), R: GCAGTCATCCAGAGATGTGACC (SEQ ID NO: 39); hGAPDH: F: ATGGACACGCTCCCCTGACT (SEQ ID NO: 40), R: GAAAGGTGGGAGCCTCAGTC (SEQ ID NO: 41). hFGF13 isoform-specific PCR was performed using the following primers: FGF13-001 (15): F: CGAGAAATCCAACGCCTGC (SEQ ID NO: 42), R: CACCACTCGCAGACCCACAG (SEQ ID NO: 43); FGF13-002 (1U): F: GTTAAGGAAGTCGTATTCAGAGC (SEQ ID NO: 44), R: CACCACTCGCAGACCCACAG (SEQ ID NO: 45); FGF13-203 (1V): F: GATGCTTCTAAGGAGCCTCAG (SEQ ID NO: 46), R: CACCACTCGCAGACCCACAG (SEQ ID NO: 47); FGF13-202 (1Y): F: ACAGAGCCGGAAGAGCCTCAG (SEQ ID NO: 48), R: CACCACTCGCAGACCCACAG (SEQ ID NO: 49); FGF13-201, 3 (1V+1Y): F: GATGCTTCTAAGGTTCTGGAT (SEQ ID NO: 50), R: CACCACTCGCAGACCCACAG (SEQ ID NO: 51).

Expression was normalized to the GAPDH housekeeping gene and compared to the control samples. For each assay, cDNA was used from three controls, three carriers and three affected individuals unless indicated otherwise. For expression analysis of hsa-miR-504 and hsa-miR-505, the following miScript primer assays (Qiagen) were used: HsmiR-504_{—}1, Hs_miR-505_{—}1, and Hs_RNU6-2_{—}1 miScript (miScript PCR Control). Images were generated using GraphPad Prism.

Whole-Mount and Section In Situ Hybridization.

For mouse section in situ hybridization, dorsal skin isolated from Swiss Webster mice at indicated time points were harvested and embedded in OCT, where a microtome cryostat was used to generate 10 μm sections. For human studies, 12 μm hair follicle sections were used. The sense and antisense riboprobes were constructed using in vitro transcription and the DIG-labeling system (Roche). The following primers were used and recognize the core region of the FGF13 sequence: mFgf13: F: TCAAACCAAGCTGTATTTGGC (SEQ ID NO: 52), R: CTTTCAGTGGTTTGGGCAGAA (SEQ ID NO: 53); hFGF13: F: AGCCTCAGCTTAAGGGTATAG (SEQ ID NO: 54), R: CAAGAACACTGTTACCTTGAGC (SEQ ID NO: 55).

Immunofluorescence Staining on Skin Biopsies.

Sections were fixed with acetone at −20° C. for 10 minutes, washed three times in 1×PBS, and then blocked in 1.5% fish gelatin/1% BSA in 1×PBS at room temperature for one hour. The rabbit anti-FGF13 antibody recognizing the C-terminus of the protein was generously provided by Dr. Geoffrey Pitt (Duke University) and was used at a concentration of 1:400 in 1% fish gelatin/1′Y° BSA in 1×PBS. The rat anti-mouse CD200 antibody (1:100) (BD Pharmingen), guinea pig anti-human K75 (1:1000) (a gift from Lutz Langbein) and rabbit anti-human K14 (1:1000) (Covance) were diluted in 1.5% fish gelatin/1% BSA in 1×PBS. The anti-rabbit, -rat, and -guinea pig IgG isotype (Santa Cruz Biotechnologies, CA, USA) antibodies were used as primary controls at the same concentrations as the respective primary antibodies listed above. Following PBS washes, the Alexa Fluor 488 donkey anti-rabbit IgG (Molecular Probes, Invitrogen), Alexa Fluor 594 donkey anti-rat IgG (Molecular Probes, Invitrogen), and Alexa Fluor 594 goat anti-guinea pig IgG (Molecular Probes, Invitrogen) secondary antibodies were added to the cryosections at a concentration of 1:800 in 1×PBS. Sections were mounted in VECTASHIELD mounting medium with DAPI (Vector Laboratories, Burlingame, Calif., USA) and imaged using a LSM 5 laser-scanning Axio Observer Z1 confocal microscope (Carl Zeiss). For human studies, Z-stack images were taken at 10× and 20× magnifications using identical settings and consistent Z-stack intervals between slides. For mouse studies, images were taken at a 20× magnification.

Statistical Analysis.

A Student's t-test (two-tailed) was used to determine statistical significance in quantitative RT-PCR assays with a significance level (α) of 0.05. Three biological replicates were used in each analysis (unless indicated otherwise) and values represent the average of three independent experiments for the three biological replicates. Error bars represent the standard error of the mean.

Assessment of X Inactivation Skewing in Female Carriers.

The HUMARA assay was performed to determine skewing of X inactivation as previously described (R5). Genomic DNA from five female carriers was used for amplification of a differentially methylated CpG site near a polymorphic region in exon I of the Androgen Receptor (AR) gene, and PCR products were digested with the HpaII (methylation-sensitive) and RsaI (co-cutter, methylation-insensitive) restriction enzymes to distinguish between methylated and nonmethylated alleles. XCI skewing percent was determined using the method described in (R5).

RNA Sequencing in Whole Skin.

RNA sequencing (RNA-seq) was performed on whole skin from one control and one affected individual. Preparation of the cDNA library for sequencing was performed using the TruSeq kit (Illumina). In brief, 100 ng total RNA extracted from affected and control skin biopsies was purified (using polyA capture to select for mRNAs), fragmented, and converted into single-stranded cDNA using random hexamer priming. Next, the second strand was generated and double-stranded cDNA was purified using bead capture. End repair was then performed to create blunt ends, followed by adenylation of the 3′ ends (to prevent intramolecular ligation), ligation of indexing adaptors to the ends of the double-stranded cDNA, and enrichment of DNA fragments containing adaptor molecules using PCR. The resulting cDNA library was then sent to the genomics core facility at Rockefeller University to be sequenced on the Illumina HiSeq 2000 machine using single-end reads ˜50 bp, with an overall sequencing depth of ˜15 millions reads per sample.

Reads were mapped to human reference genome (NCBI build 37.2), using TopHat, an algorithm designed to align reads from an RNA-Seq to a reference genome based on existing transcripts annotation and inferred new splice sites on the fly (R6). To estimate the relative abundance of genes and splice isoforms, the data were then analyzed using Cufflinks, a program that contains algorithms that estimate transcript abundance, while accounting for alternative splicing (R7). Fragments per kb of exon per million fragments mapped (FPKM) were normalized to the upper quartile. Differential expression of isoforms was tested using Cuffdiff, a program that utilizes the Cufflinks transcript quantification engine to determine transcript levels in more than one condition.

In Silico Prediction Analysis of miR-504 Target Genes.

miR-504 target genes were determined using a comprehensive database, miRWalk (R8), which provides information on predicted, validated and published miRNA target genes. We applied filters to select target genes with a minimum seed sequence of 7 nucleotides, the longest transcript of a given gene, and ap value of 0.05 of less, which represents the strength of the prediction through a Poisson distribution. Furthermore, target genes that appeared in three or more of the following prediction programs were selected: TargetScan, miRanda, miRDB, PICTAR5, miRWalk, RNA22, and DIANA-mT.

### REFERENCES

P1. Beighton P (1970) Congenital hypertrichosis lanuginosa. Arch Dermatol 101(6):669-672.
P2. Garcia-Cruz D, Figuera L E, & Cantu J M (2002) Inherited hypertrichoses. Clinical Genetics 61(5):321-329.
P3. Cantu J M & Ruiz C (1985) On atavisms and atavistic genes. Ann Genet 28(3):141-142.
P4. Hall B K (1984) Development mechanisms underlying the formation of atavisms. Biol Rev Camb Philos Soc 59(1):89-124.
P5. Kim J, et al. (2010) Ambras syndrome in a Korean patient with balanced pericentric inversion (8)(p11.2q24.2). J Dermatol Sci 59(3):204-206.
P6. Tadin M, et al. (2001) Complex cytogenetic rearrangement of chromosome 8q in a case of Ambras syndrome. American Journal of Medical Genetics 102(1):100-104.
P7. Sun M, et al. (2009) Copy-number mutations on chromosome 17q24.2-q24.3 in congenital generalized hypertrichosis terminalis with or without gingival hyperplasia. Am J Hum Genet 84(6):807-813.
P8. Fantauzzo K A, et al. (2008) A position effect on TRPS1 is associated with Ambras syndrome in humans and the Koala phenotype in mice. Hum Mol Genet 17(22):3539-3551.
P9, Fantauzzo K A, Kurban M, Levy B, & Christiana A M (2012) Trps1 and its target gene sox9 regulate epithelial proliferation in the developing hair follicle and are associated with hypertrichosis. PLoS Genet 8(11).
P10. Kleinjan D J & van Heyningen V (1998) Position effect in human genetic disease. Hum Mol Genet 7(10):1611-1618.
P11. Tadin-Strapps M, et al. (2003) Congenital universal hypertrichosis with deafness and dental anomalies inherited as an X-linked trait. Clinical Genetics 63(5):418-422.
P12. Plenge R M, Stevenson R A, Lubs H A, Schwartz C E, & Willard H F (2002) Skewed X-chromosome inactivation is a common feature of X-linked mental retardation disorders. Am J Hum Genet 71(1):168-173.
P13. Knudsen G P, et al, (2006) Increased skewing of X chromosome inactivation in Rett syndrome patients and their mothers. Eur J Hum Genet 14(11):1189-1194.
P14. Zhu H, et al. (2011) X-linked congenital hypertrichosis syndrome is associated with interchromosomal insertions mediated by a human-specific palindrome near SOX3. American Journal of Human Genetics 88(6):819-826.
P15. Ohyama M, et al. (2006) Characterization and isolation of stem cell-enriched human hair follicle bulge cells. Journal of Clinical Investigation 116(1):249-260.
P16. Kawano M, Suzuki S, Suzuki M, Oki J, & Imamura T (2004) Bulge- and basal layer-specific expression of fibroblast growth factor-13 (FHF-2) in mouse skin. J Invest Dermatol 122(5):1084-1090.
P17. Lettice L A, et al. (2003) A long-range Shh enhancer regulates expression in the developing limb and fin and is associated with preaxial polydactyly. Hum Mol Genet 12(14):1725-1735.
P18. Lettice L A, et al. (2002) Disruption of a long-range cis-acting regulator for Shh causes preaxial polydactyly. Proc Natl Acad Sci USA 99(11):7548-7553.
P19. Leipoldt M, et al. (2007) Two novel translocation breakpoints upstream of SOX9 define borders of the proximal and distal breakpoint cluster region in campomelic dysplasia. Clin Genet 71(1):67-75.
P20. Velagaleti G V, et al. (2005) Position effects due to chromosome breakpoints that map approximately 900 Kb upstream and approximately 1.3 Mb downstream of SOX9 in two patients with campomelic dysplasia. Am J Hum Genet 76(4):652-662.
P21. Millar S E, et al. (1999) WNT signaling in the control of hair growth and structure. Dev Biol 207(1):133-149.
P22. Yano K, Brown L F, & Detmar M (2001) Control of hair growth and follicle size by VEGF-mediated angiogenesis. J Clin Invest 107(4):409-417.
P23. Kettunen P, Furmanek T, Chaulagain R, Kvinnsland I R, & Luukko K (2011) Developmentally regulated expression of intracellular Fgf11-13, hormone-like Fgf15 and canonical Fgf16, -17 and -20 mRNAs in the developing mouse molar tooth. Acta Odontol Scand 69(6):360-366.
P24. Schoorlemmer J & Goldfarb M (2002) Fibroblast growth factor homologous factors and the islet brain-2 scaffold protein regulate activation of a stress-activated protein kinase. J Biol Chem 277(51):49111-49119.
P25. Hebert J M, Rosenquist T, Gotz J, & Martin G R (1994) FGF5 as a regulator of the hair growth cycle: evidence from targeted and spontaneous mutations. Cell 78(6):1017-1025.
P26. Housley D J & Venta P J (2006) The long and the short of it: evidence that FGF5 is a major determinant of canine ‘hair’-itability. Anim Genet 37(4):309-315.
P27. Liu H Y, Yang G Q, Zhang W, Zhu XP, & Jia Z H (2009) [Effects of FGF5 gene on fibre traits on Inner Mongolian cashmere goats]. Yi Chuan 31(2):175-179.
P28. Li C X, Jiang M S, Chen S Y, & Lai S J (2008) [Correlation analysis between single nucleotide polymorphism of FGF5 gene and wool yield in rabbits]. Yi Chuan 30(7):893-899.
P29. Wu Q F, et al. Fibroblast growth factor 13 is a microtubule-stabilizing protein regulating neuronal polarization and migration. Cell 149(7):1549-1564.
P30. Tyson R, Graham J P, Colahan P T, & Berry C R (2004) Skeletal atavism in a miniature horse. Vet Radiol Ultrasoun 45(4):315-317.
P31. Walia I, Arora H S, Barker E A, Delgado R M, & Frazier O H (2010) Snake Heart A Case of Atavism in a Human Being. Tex Heart I J 37(0:687-690.
P32. Peterkova R, Lesot H, & Peterka M (2006) Phylogenetic memory of developing mammalian dentition. J Exp Zool Part B 306B(3):234-250.

### SUPPLEMENTARY REFERENCES

R1. Li H & Durbin R (2009) Fast and accurate short read alignment with Burrows-Wheeler transform. Bioinformatics 25(14):1754-1760.
R2. Li H, et al. (2009) The Sequence Alignment/Map format and SAMtools. Bioinformatics 25(16):2078-2079.
R3. Ge D, et al. (2011) SVA: software for annotating and visualizing sequenced human genomes. Bioinformatics 27(14):1998-2000.
R4. Zhu H, et al. (2011) X-linked congenital hypertrichosis syndrome is associated with interchromosomal insertions mediated by a human-specific palindrome near SOX3. American Journal of Human Genetics 88(6):819-826.
R5. Warburton D, et al. (2009) Skewed X chromosome inactivation and trisomic spontaneous abortion: no association. Am J Hum Genet 85(2):179-193.
R6. Trapnell C, Pachter L, & Salzberg S L (2009) TopHat: discovering splice junctions with RNA-Seq. Bioinformatics 25(9):1105-1111.
R7. Trapnell C, et al. (2010) Transcript assembly and quantification by RNA-Seq reveals unannotated transcripts and isoform switching during cell differentiation. Nat Biotechnol 28(5):511-515.
R8. Dweep H, Sticht C, Pandey P, & Gretz N (miRWalk—database: prediction of possible miRNA binding sites by “walking” the genes of three genomes. J Biomed Inform 44(5):839-847.

What is claimed:

1. A method for inducing hair growth in a subject, the method comprising:
(a) administering to the subject an effective amount of an inhibitor of FGF13,
thereby inducing hair growth in the subject.

2. The method of claim 1 wherein the subject is a mammal.

3. The method of claim 1, wherein the inhibitor comprises an antibody that specifically binds to a protein comprising SEQ ID NO: 1, 3, 5, 7, 9, or 11.

4. The method of claim 2, wherein the subject is afflicted with a hair-loss disorder.

5. The method of claim 4, wherein the hair-loss disorder comprises androgenetic alopecia, telogen effluvium, alopecia areata, tinea capitis, alopecia totalis, hypotrichosis, hereditary hypotrichosis simplex, or alopecia universalis.

6. The method of claim 1, further comprising the step (b) determining whether the inhibitor administered induced hair growth in the subject as compared to the subject's hair growth prior to treatment with the inhibitor.

7. The method of claim 6, wherein the subject is afflicted with a hair loss disorder.

8. The method of claim 1, wherein the inhibitor comprises an antisense RNA that specifically inhibits expression of the gene that encodes the FGF13 protein; a siRNA that specifically targets the gene that encodes the FGF13 protein; or a small molecule that specifically inhibits the expression or activity of FGF13.

9. The method of claim 8, wherein the siRNA is directed to a human nucleic acid sequence comprising SEQ ID NO: 2, 4, 6, 8, 10, or 12.

10. The method of claim 8, wherein the siRNA directed to a FGF13 gene is any one of the sequences listed in Table 1.

11. A method for reducing hair growth in a subject, the method comprising:
(a) administering to the subject an effective amount of FGF13 protein or an activator of FGF13 expression,
thereby reducing hair growth in the subject.

12. The method of claim 11, wherein the subject is a mammal.

13. The method of claim 11, wherein the subject is afflicted with a hair-growth disorder.

14. The method of claim 14, wherein the hair-growth disorder comprises X-linked hypertrichosis, generalized hypertrichosis terminalis with or without gingival hyperplasia, autosomal recessive hypertrichosis, Cantu syndrome, Ambras type hypertrichosis, autosomal recessive trichomegaly or a combination thereof.

15. The method of claim 11, further comprising the step of (b) determining whether the activator administered reduced hair growth in the subject as compared to the subject's hair growth prior to treatment with the activator.

16. The method of claim 11, wherein the activator is a polypeptide comprising SEQ ID NO: 1, 3, 5, 7, 9, or 11, or a fragment thereof; or a peptidomimetic comprising SEQ ID NO: 1, 3, 5, 7, 9, or 11.

17. A method for determining the presence of or a predisposition to developing a hair-growth disorder in a subject, the method comprising:
(a) extracting a sample from a subject;
(b) detecting the presence or reduction of an FGF13 protein in the subject as compared to a subject not afflicted with a hair growth disorder, wherein the reduction of the FGF13 protein is indicative of a hair growth disorder.

18. The method of claim 17 further comprising incubating the sample with an agent that binds an FGFG13 protein, or fragment thereof.

19. The method of claim 18, wherein the agent is an antibody that specifically binds to the FGF13 protein, or fragment thereof.

20. The method of claim 17, wherein the hair-growth disorder comprises X-linked hypertrichosis, generalized hypertrichosis terminalis with or without gingival hyperplasia, autosomal recessive hypertrichosis, Cantu syndrome, Ambras type hypertrichosis, autosomal recessive trichomegaly or a combination thereof.

21. A method for determining the presence of or a predisposition to developing a hair-growth disorder in a subject, the method comprising:
(a) extracting a sample from a subject;
(b) detecting the presence or reduction of a FGF13 nucleic acid as compared to a subject not afflicted with a hair growth disorder, wherein the reduction of the FGF13 nucleic acid is indicative of a hair growth disorder.

22. The method of claim 21, wherein the detecting comprises using PCR and primers directed to SEQ ID NO: 2, 4, 6, 8, 10, or 12.

23. The method of claim 22, wherein one or more of the primers comprises SEQ ID NO: 24, 25, 26, 27, 28, 29, 54, 55.

24. The method of claim 21, wherein the hair-growth disorder comprises X-linked hypertrichosis, generalized hypertrichosis terminalis with or without gingival hyperplasia, autosomal recessive hypertrichosis, Cantu syndrome, Ambras type hypertrichosis, autosomal recessive trichomegaly or a combination thereof.
